SEQUENCE LISTING (26 sequences)

SEQ ID NO: 1 - DNA, 27 bp, Synthetic construct
  source 1..27: mol_type = other DNA
agcttggcgtaatcatggtcatagctg

SEQ ID NO: 2 - DNA, 40 bp, Synthetic construct
  source 1..40: mol_type = other DNA
gatcggcacgtaagaggggacttccattgttcattccacg

SEQ ID NO: 3 - DNA, 40 bp, Synthetic construct
  source 1..40: mol_type = other DNA
aatgaacaatggaagtcccctcttacgtgccgatcaagtc

SEQ ID NO: 4 - DNA, 27 bp, Synthetic construct
  source 1..27: mol_type = other DNA
gggttagcggttatcaacctcccaact

SEQ ID NO: 5 - DNA, 40 bp, Synthetic construct; identical to SEQ ID NO: 3
  source 1..40: mol_type = other DNA

SEQ ID NO: 6 - DNA, 38 bp, Synthetic construct
  source 1..38: mol_type = other DNA
cggttcactaaacgagctctgcttatatagacctccca

SEQ ID NO: 7 - DNA, 40 bp, Synthetic construct
  source 1..40: mol_type = other DNA
gcagagctcgtttagtgaaccgagttgttgatctgtgtga

SEQ ID NO: 8 - DNA, 25 bp, Synthetic construct
  source 1..25: mol_type = other DNA
ggaccttgcaaggttccagctcaga

SEQ ID NO: 9 - DNA, 28 bp, Synthetic construct
  source 1..28: mol_type = other DNA
gtgtgaaataccccgaacccatgatcct

SEQ ID NO: 10 - RNA, 50 nt, Synthetic construct
  source 1..50: mol_type = other RNA
cgttccaaccactgactcgaaagagtcagtggttggaacgcgcaggtgcc

SEQ ID NO: 11 - DNA, 599 bp, Synthetic construct
  source 1..599: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagtaatacgactcactattag

SEQ ID NO: 12 - DNA, 599 bp, Synthetic construct
  source 1..599: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagtaatacgactcactatagg

SEQ ID NO: 13 - DNA, 54 bp, Synthetic construct
  source 1..54: mol_type = other DNA
gaaggaagaggaagtctgctaacatgcggtgacgtcgaggagaatcctggccca

SEQ ID NO: 14 - DNA, 48 bp, Synthetic construct
  source 1..48: mol_type = other DNA
atgaagaccatcatcgccctgagctacatcttctgcctggtgttcgcc

SEQ ID NO: 15 - DNA, 1236 bp, Synthetic construct
  source 1..1236: mol_type = other DNA
tacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaag

SEQ ID NO: 16 - DNA, 1089 bp, Synthetic construct
  source 1..1089: mol_type = other DNA
agcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtac

SEQ ID NO: 17 - DNA, 57 bp, Synthetic construct
  source 1..57: mol_type = other DNA
ggcggtggaggctcaggaggtggcggtagcggtggcggaggttcaggtggtggtgga

SEQ ID NO: 18 - DNA, 2430 bp, Synthetic construct
  source 1..2430: mol_type = other DNA
atgaagaccatcatcgccctgagctacatcttctgcctggtgttcgccagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtacggcggtggaggctcaggaggtggcggtagcggtggcggaggttcaggtggtggtggatacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaag

SEQ ID NO: 19 - DNA, 12781 bp, Synthetic construct
  source 1..12781: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagtaatacgactcactattagttgttgatctgtgtgaatcagactgcgacagttcgagtttgaagcgaaagctagcaacagtatcaacaggttttattttggatttggaaacgagagtttctggtccgttccaaccactgactcgaaagagtcagtggttggaacgcgcaggtgccatgaaaaacccaaagaagaaatccggaggattccggattgtcaatatgctaaaacgcggagtagcccgtgtgagcccctttgggggcttgaagaggctgccagccggacttctgctgggtcatgggcccatcaggatggtcttggcgattgaaggaagaggaagtctgctaacatgcggtgacgtcgaggagaatcctggcccaatgaagaccatcatcgccctgagctacatcttctgcctggtgttcgccagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtacggcggtggaggctcaggaggtggcggtagcggtggcggaggttcaggtggtggtggatacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagggatccggtgggtccggtagtggtggttccaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaaaggtattgattttaaagaagatggaaacattctcggacacaaactggagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggacgagctgtacaaggaaggaagaggaagtctgctaacatgcggtgacgtcgaggagaatcctggcccattgggtctgaatacaaagaatggatctatttcccttatgtgcttggccttagggggagtgttgatcttcttatccacagccgtctctgctgatgtggggtgctcggtggacttctcaaagaaggaaacgagatgcggtacaggggtgttcgtctataacgacgttgaagcttggagggacaggtacaagtaccatcctgactcccctcgtagattggcagcagcagtcaagcaagcctgggaagatgggatctgtgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccatggctggaaggcttgggggaaatcgtacttcgtcagggcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgtcattggaacagccgctaagggaaaggaggctgtgcacagtgatctaggctactggattgagagtgagaagaacgacacatggaggctgaagagggcccacctgatcgagatgaaaacatgtgaatggccaaagtcccacacattgtggacagatggaatagaagaaagtgatctgatcatacccaagtctttagctgggccactcagccatcacaacaccagagagggctacaggacccaaatgaaagggccatggcatagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggttgttggtatggaatggagataaggcccaggaaagaaccagaaagtaacttagtaaggtcaatggtgactgcaggatcaactgatcacatggatcacttctcccttggagtgcttgtgattctgctcatggtacaggaagggctaaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgttgctcatctggcgctgatagcggcattcaaagtcagacctgcgttgctggtatctttcattttcagagctaattggacaccccgtgagagcatgctgctggccttggcctcgtgtcttctgcaaactgcgatctccgccttggaaggcgacctgatggttcccatcaatggttttgctttggcctggttggcaatacgagcgatggttgttccacgcactgacaacatcaccttggcaatcctggctgctctgacaccactggcccggggcacactgcttgtggcgtggagagcaggccttgctacttgcggggggttcatgctcctttctctgaaggggaaaggcagtgtgaagaagaacttaccatttgtcatggccctgggactaaccgctgtgaggctggtcgaccccatcaacgtggtgggactgctgttgctcacaaggagtgggaagcggagctggccccctagtgaagtactcacagctgttggcctgatatgcgcattggctggagggttcgccaaggcggatatagagatggctgggcccatggccgcggtcggtctgctaattgtcagttacgtggtcgcaggaaagagtgtggacatgtacattgaaagagcaggtgacatcacatgggaaaaagatgcggaagtcactggaaacagtccccggctcgatgtggcactagatgagagtggtgatttctccctagtggaggatgatggtccccccatgagagagatcatactcaaagtggtcctgatggccatctgtggcatgaacccaatagccataccctttgcagctggagcgtggtacgtgtatgtgaagactggaaaaaggagtggtgctctatgggatgtgcctgctcccaaggaagtaaaaaagggggagaccacagatggagtgtacagagtaatgactcgtagactgctaggttcaacacaagttggagtgggagtcatgcaagagggggtcttccacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcagttgcgctggactacccagcaggaacttcaggatctccaatcctagataagtgtgggagagtgataggactctatggtaatggggtcgtgatcaaaaatgggagttacgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgcactgtgatcttagctccaaccagggttgtcgctgctgaaatggaggaagcccttagagggcttccagtgcgttatatgacaacagcagtcaatgtcacccattctgggacagaaatcgttgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgactgccacgccaccaggaacccgtgacgcattcccggactccaactcaccaattatggacaccgaagtggaagtcccagagagagcctggagctcaggctttgattgggtgacggatcattctggaaaaacagtttggtttgttccaagcgtgaggaatggcaatgagatcgcagcttgtctgacaaaggctggaaaacgggtcatacagctcagcagaaagacttttgagacagagttccagaaaacaaaacatcaagagtgggacttcgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaaccccaacaaacctggagatgagtatctgtatggaggtgggtgcgcagagactgatgaagaccatgcacactggcttgaagcaagaatgcttcttgacaacatttacctccaagatggcctcatagcctcgctctatcgacctgaggccgacaaagtagcagctattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagatacggagagaaaagagtgctcaaaccgaggtggatggacgccagagtttgttcagatcatgcggccctgaagtcattcaaagagtttgccgctgggaaaagaggagcggcctttggagtgatggaagccctgggaacactgccaggacatatgacagagagattccaggaggccattgacaacctcgctgtgctcatgcgggcagagactggaagcaggccctacaaagccgcggcggcccaattaccggagaccctagagactatcatgcttttggggttgctgggaacagtctcgctgggaatctttttcgtcttgatgcggaacaagggcatagggaagatgggctttggaatggtgactcttggggccagcgcatggcttatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtgggtcttctgggcttgattaccgccaatgaactcggatggttggagagaacaaagagtgacctaagccatctaatgggaaggagagaggagggggcaactataggattctcaatggacattgacctgcggccagcctcagcttgggctatctatgctgctctgacaactttcattaccccagccgtccaacatgcagtgaccacttcatacaacaactactccttaatggcgatggccacgcaagctggagtgttgttcggtatgggtaaagggatgccattctatgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagctgtctccagcgccatactgtcgcggaccgcctgggggtggggtgaggctggggccctgatcacagctgcaacttccactttgtgggagggctctccgaacaagtactggaactcctccacagccacctcactgtgtaacatttttaggggaagctacttggctggagcttctctaatctacacagtaacaagaaacgctggcttggtcaagagacgtgggggtggaacgggagagaccctgggagagaaatggaaggcccgcctgaaccagatgtcggccctggagttctactcctacaaaaagtcaggcatcaccgaggtgtgcagagaagaggcccgccgcgccctcaaggacggtgtggcaacgggaggccacgctgtgtcccgaggaagtgcaaagctgagatggttggtggagaggggatacctgcagccctatggaaaggtcattgatcttggatgtggcagagggggctggagttactatgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgatataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctccttttggggcgcatggacgggcccaggaggccagtgaaatatgaagaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctacgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagttaggcaaacacaaacggccacgagtctgtaccaaagaagagttcatcaacaaggttcgtagcaacgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtacatgtggctaggggctagatttctagagttcgaagcccttggattcttgaacgaggatcactggatggggagagagaattcaggaggtggtgttgaagggctaggattacaaagactcggatatgtcttagaagagatgagtcgcataccaggaggaaggatgtatgcagatgatactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacatttaccaacctagtggtgcagctcattcggaatatggaggctgaggaagttctagagatgcaagacttgtggctgctgcggaggtcagagaaagtgaccaactggttgcagagcaatggatgggataggctcaaacgaatggcagtcagtggagatgattgcgttgtgaaaccaattgatgataggtttgcacatgctctcaggttcttgaatgatatgggaaaagttaggaaggacacacaagagtggaagccctcaactggatgggacaactgggaagaagttccgttttgctcccaccacttcaacaagctccatctcaaggacgggaggtccattgtggttccctgccgccaccaagatgaactgattggccgagctcgcgtctcaccgggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtgggtctctcatagggcacagaccgcgcaccacctgggctgagaacattaaaaacacagtcaacatgatgcgtaggatcataggtgatgaagaaaagtacgtggactacctatccacccaagttcgctacttgggcgaagaagggtccacacctggagtgctagcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtctgggtcggcatggcatctccacctcctcgcggtccgacctgggctacttcggtaggctaagggagaagccaacttgtttattgcagcttataatggttacaaataaagcaatagcatcacaaatttcacaaataaagcatttttttcactgcattctagttgtggtttgtccaaactcatcaatgtatcttatcatg

SEQ ID NO: 20 - DNA, 597 bp, Synthetic construct
  source 1..597: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagtaatacgactcactatt

SEQ ID NO: 21 - DNA, 307 bp, Synthetic construct
  source 1..307: mol_type = other DNA
agttgttgatctgtgtgaatcagactgcgacagttcgagtttgaagcgaaagctagcaacagtatcaacaggttttattttggatttggaaacgagagtttctggtccgttccaaccactgactcgaaagagtcagtggttggaacgcgcaggtgccatgaaaaacccaaagaagaaatccggaggattccggattgtcaatatgctaaaacgcggagtagcccgtgtgagcccctttgggggcttgaagaggctgccagccggacttctgctgggtcatgggcccatcaggatggtcttggcgatt

SEQ ID NO: 22 - DNA, 8472 bp, Synthetic construct
  source 1..8472: mol_type = other DNA
ttgggtctgaatacaaagaatggatctatttcccttatgtgcttggccttagggggagtgttgatcttcttatccacagccgtctctgctgatgtggggtgctcggtggacttctcaaagaaggaaacgagatgcggtacaggggtgttcgtctataacgacgttgaagcttggagggacaggtacaagtaccatcctgactcccctcgtagattggcagcagcagtcaagcaagcctgggaagatgggatctgtgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccatggctggaaggcttgggggaaatcgtacttcgtcagggcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgtcattggaacagccgctaagggaaaggaggctgtgcacagtgatctaggctactggattgagagtgagaagaacgacacatggaggctgaagagggcccacctgatcgagatgaaaacatgtgaatggccaaagtcccacacattgtggacagatggaatagaagaaagtgatctgatcatacccaagtctttagctgggccactcagccatcacaacaccagagagggctacaggacccaaatgaaagggccatggcatagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggttgttggtatggaatggagataaggcccaggaaagaaccagaaagtaacttagtaaggtcaatggtgactgcaggatcaactgatcacatggatcacttctcccttggagtgcttgtgattctgctcatggtacaggaagggctaaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgttgctcatctggcgctgatagcggcattcaaagtcagacctgcgttgctggtatctttcattttcagagctaattggacaccccgtgagagcatgctgctggccttggcctcgtgtcttctgcaaactgcgatctccgccttggaaggcgacctgatggttcccatcaatggttttgctttggcctggttggcaatacgagcgatggttgttccacgcactgacaacatcaccttggcaatcctggctgctctgacaccactggcccggggcacactgcttgtggcgtggagagcaggccttgctacttgcggggggttcatgctcctttctctgaaggggaaaggcagtgtgaagaagaacttaccatttgtcatggccctgggactaaccgctgtgaggctggtcgaccccatcaacgtggtgggactgctgttgctcacaaggagtgggaagcggagctggccccctagtgaagtactcacagctgttggcctgatatgcgcattggctggagggttcgccaaggcggatatagagatggctgggcccatggccgcggtcggtctgctaattgtcagttacgtggtcgcaggaaagagtgtggacatgtacattgaaagagcaggtgacatcacatgggaaaaagatgcggaagtcactggaaacagtccccggctcgatgtggcactagatgagagtggtgatttctccctagtggaggatgatggtccccccatgagagagatcatactcaaagtggtcctgatggccatctgtggcatgaacccaatagccataccctttgcagctggagcgtggtacgtgtatgtgaagactggaaaaaggagtggtgctctatgggatgtgcctgctcccaaggaagtaaaaaagggggagaccacagatggagtgtacagagtaatgactcgtagactgctaggttcaacacaagttggagtgggagtcatgcaagagggggtcttccacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcagttgcgctggactacccagcaggaacttcaggatctccaatcctagataagtgtgggagagtgataggactctatggtaatggggtcgtgatcaaaaatgggagttacgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgcactgtgatcttagctccaaccagggttgtcgctgctgaaatggaggaagcccttagagggcttccagtgcgttatatgacaacagcagtcaatgtcacccattctgggacagaaatcgttgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgactgccacgccaccaggaacccgtgacgcattcccggactccaactcaccaattatggacaccgaagtggaagtcccagagagagcctggagctcaggctttgattgggtgacggatcattctggaaaaacagtttggtttgttccaagcgtgaggaatggcaatgagatcgcagcttgtctgacaaaggctggaaaacgggtcatacagctcagcagaaagacttttgagacagagttccagaaaacaaaacatcaagagtgggacttcgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaaccccaacaaacctggagatgagtatctgtatggaggtgggtgcgcagagactgatgaagaccatgcacactggcttgaagcaagaatgcttcttgacaacatttacctccaagatggcctcatagcctcgctctatcgacctgaggccgacaaagtagcagctattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagatacggagagaaaagagtgctcaaaccgaggtggatggacgccagagtttgttcagatcatgcggccctgaagtcattcaaagagtttgccgctgggaaaagaggagcggcctttggagtgatggaagccctgggaacactgccaggacatatgacagagagattccaggaggccattgacaacctcgctgtgctcatgcgggcagagactggaagcaggccctacaaagccgcggcggcccaattaccggagaccctagagactatcatgcttttggggttgctgggaacagtctcgctgggaatctttttcgtcttgatgcggaacaagggcatagggaagatgggctttggaatggtgactcttggggccagcgcatggcttatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtgggtcttctgggcttgattaccgccaatgaactcggatggttggagagaacaaagagtgacctaagccatctaatgggaaggagagaggagggggcaactataggattctcaatggacattgacctgcggccagcctcagcttgggctatctatgctgctctgacaactttcattaccccagccgtccaacatgcagtgaccacttcatacaacaactactccttaatggcgatggccacgcaagctggagtgttgttcggtatgggtaaagggatgccattctatgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagctgtctccagcgccatactgtcgcggaccgcctgggggtggggtgaggctggggccctgatcacagctgcaacttccactttgtgggagggctctccgaacaagtactggaactcctccacagccacctcactgtgtaacatttttaggggaagctacttggctggagcttctctaatctacacagtaacaagaaacgctggcttggtcaagagacgtgggggtggaacgggagagaccctgggagagaaatggaaggcccgcctgaaccagatgtcggccctggagttctactcctacaaaaagtcaggcatcaccgaggtgtgcagagaagaggcccgccgcgccctcaaggacggtgtggcaacgggaggccacgctgtgtcccgaggaagtgcaaagctgagatggttggtggagaggggatacctgcagccctatggaaaggtcattgatcttggatgtggcagagggggctggagttactatgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgatataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctccttttggggcgcatggacgggcccaggaggccagtgaaatatgaagaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctacgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagttaggcaaacacaaacggccacgagtctgtaccaaagaagagttcatcaacaaggttcgtagcaacgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtacatgtggctaggggctagatttctagagttcgaagcccttggattcttgaacgaggatcactggatggggagagagaattcaggaggtggtgttgaagggctaggattacaaagactcggatatgtcttagaagagatgagtcgcataccaggaggaaggatgtatgcagatgatactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacatttaccaacctagtggtgcagctcattcggaatatggaggctgaggaagttctagagatgcaagacttgtggctgctgcggaggtcagagaaagtgaccaactggttgcagagcaatggatgggataggctcaaacgaatggcagtcagtggagatgattgcgttgtgaaaccaattgatgataggtttgcacatgctctcaggttcttgaatgatatgggaaaagttaggaaggacacacaagagtggaagccctcaactggatgggacaactgggaagaagttccgttttgctcccaccacttcaacaagctccatctcaaggacgggaggtccattgtggttccctgccgccaccaagatgaactgattggccgagctcgcgtctcaccgggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtgggtctctcatagggcacagaccgcgcaccacctgggctgagaacattaaaaacacagtcaacatgatgcgtaggatcataggtgatgaagaaaagtacgtggactacctatccacccaagttcgctacttgggcgaagaagggtccacacctggagtgctagcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtctgggtcggcatggcatctccacctcctcgcggtccgacctgggctacttcggtaggctaagggagaag

SEQ ID NO: 23 - DNA, 7995 bp, Synthetic construct
  source 1..7995: mol_type = other DNA
ccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtcccctcttacgtgccgatcaagtcaaaagcctccggtcggaggcttttgactttctgctatggaggtcaggtatgatttaaatggtcagtattgagcgatatctagagaattcgtcgaagaatctgcttagggttaggcgttttgcgctgcttcgcgatgtacgggccagatatacgcgtt

SEQ ID NO: 24 - DNA, 738 bp, Synthetic construct
  source 1..738: mol_type = other DNA
ggatccggtgggtccggtagtggtggttccaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaaaggtattgattttaaagaagatggaaacattctcggacacaaactggagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggacgagctgtacaag

SEQ ID NO: 25 - RNA, 38 nt, Synthetic construct
  source 1..38: mol_type = other RNA
cgaccacggccggaaacggccgtggtcgcgcaggtgcc

SEQ ID NO: 26 - RNA, 17 nt, Synthetic construct
  source 1..17: mol_type = other RNA
cgtacgagcgcaggtgc

(19) United States
(12) Patent Application Publication
(10) Pub. No.: US 2025/0255952 A1
(43) Pub. Date: Aug. 14, 2025

(54) SELF-AMPLIFYING mRNA VACCINE BASED ON Z7 GENOME

(71) Applicants: Fengwei Bai, Hattiesburg, MS (US); Faqing Huang, Hattiesburg, MS (US)

(72) Inventors: Fengwei Bai, Hattiesburg, MS (US); Faqing Huang, Hattiesburg, MS (US)

(73) Assignee: The University of Southern Mississippi, Hattiesburg, MS (US)

(21) Appl. No.: 19/050,556

(22) Filed: Feb. 11, 2025

Related U.S. Application Data
(60) Provisional application No. 63/552,625, filed on Feb. 12, 2024.

(51) Int. Cl. A61K 39/12; A61K 39/00; A61P 37/04; C07K 14/005; C12N 7/00

(52) U.S. Cl. CPC A61K 39/12; A61P 37/04; C07K 14/005; C12N 7/00; A61K 2039/5254; C12N 2770/24121; C12N 2770/24162; C12N 2770/36122; C12N 2770/36134; C12N 2770/36171

(57) ABSTRACT

The present invention relates to a self-amplifying mRNA vaccine, including: a self-amplifying backbone comprising a live attenuated virus having a hairpin loop insert, and wherein at least one or more structural genes of the live attenuated virus have been replaced with at least one target antigen-encoding nucleic acid sequence.

### CROSS REFERENCE TO RELATED APPLICATIONS

This application claims the benefit of U.S. provisional application No. 63/552,625, filed on Feb. 12, 2024, the disclosure of which is hereby incorporated by reference in its entirety as if fully set forth herein.

### REFERENCE TO XML SEQUENCE LISTING

This application includes a sequence listing submitted herewith as an XML file named USM-1019USPSequenceListingXML” created on Feb. 9, 2024, and containing 41,000 bytes. The material contained in this XML file is incorporated herein by reference.

### BACKGROUND OF THE INVENTION

Chikungunya virus (CHIKV) is a mosquito-transmitted, single-strand, positive-sense RNA alphavirus (AV) belonging to the Togaviridae family. CHIKV was first discovered in 1953 in Africa, and from 2004 to 2011, 1.4 to 6.5 million CHIKV cases were confirmed from nearly 40 countries in Africa, Asia, and Europe[244]. In 2014, CHIKV disease cases were reported among U.S. travelers, and local transmission was also identified in Florida, Texas, and Puerto Rico. Approximately 580,000 suspected and laboratory-confirmed cases were reported in the Americas in 2014 [245]. The recent re-emergence of CHIKV was correlated with the rapid spread of one of its mosquito vectors Aedes albopictus and the increased fitness of CHIKV in this mosquito species[246]. Although native to warm, tropical regions of Asia, A. albopictus has successfully adapted to cooler climates, including North America. According to the CDC, A. albopictus now circulates in 866 counties of 26 states in the U.S., and there are many travel-related cases [247], indicating a potential epidemic risk of CHIKV in the U.S. The typical clinical symptoms include high fever, headache, maculopapular rashes, myalgia, edema of the extremities, and gastrointestinal complaints[248]. CHIKV often replicates in joints and results in painful inflammation and swelling, which may develop into persistent chronic arthritis[249]. Besides arthritis, CHIKV infections also cause life-threatening clinical manifestations, including neurological and cardiovascular complications[250]. However, vaccines or specific drugs against CHIKV are urgently needed.

Compared with other types of vaccines, mRNA-based vaccines are relatively safe and easy to prepare and can be manufactured on a large scale. The success of mRNA vaccines in the COVID-19 pandemic demonstrated mRNA as a new and effective vaccine platform with great potential for developing vaccines, other infectious agents, and possibly even some cancers. mRNA-based vaccination can be achieved using different approaches. For example, mRNA-based vaccination can be achieved by:
1) synthetic non-amplifying mRNA (NAM, e.g., Moderna's mRNA-1273 and the BioNTech, Pfizer vaccines for COVID-19), and
2) non-transmissible self-amplifying mRNA (SAM) based on the RNA replicon of a single-stranded (ss) RNA virus whose structural genes are replaced with the target antigen sequence. In the case of SAM, the target antigen containing the RNA replicon can efficiently replicate without producing infectious viral particles.

Both types of RNA molecules need to be formulated with delivery systems such as polymers and polymer/lipid nanoparticles (NP) for effective protection of the RNA and transfection of the RNA into cells. SAM vaccines can elicit stronger humoral and cellular responses at much lower doses than NAM vaccines, while significantly reducing potentially harmful innate immune responses mediated by cellular pattern recognition receptors upon transfection of exogenous mRNA. Therefore, SAM replicon based approaches are more robust than NAM approaches for the development of potent and safe vaccines.

Different types of self-amplifying mRNA (SAM) systems have already been developed, mainly based on the alphavirus (AV) family, including the Sindbis virus, the Semliki Forest virus, and the Venezuelan equine encephalitis virus (VEE). Flavivirus (FV) family-based RNA replicons have also been developed, including systems based on the Kunjin virus (KUNV), the classical swine fever virus (CSFV), the dengue virus (DENV), the West Nile virus (WNV), and the yellow fever virus (YFV). In addition, a Zika virus (ZIKV) replicon has been recently constructed to express luciferase.

The major advantages of FV replicons over AV replicons are that FV replicons possess high fidelity of RNA amplification and have a low recombination tendency. Moreover, FV-based systems may improve vaccine efficacy by upregulating MHC I.

Type I interferon (IFN) and other innate immune responses have been documented to be detrimental to either NAM or SAM vaccines[53, 54]. Type I IFN has been shown to significantly inhibit antibody production in the vaccination of VEEV-based SAM in mice[55]. Therefore, uridines have been replaced with pseudouridines (ψ) in the FDA-approved mRNA vaccines for COVID-19 to mitigate the harmful innate immune responses[54]. However, this strategy may not work in SAM vaccines since W replacement may inhibit SAM replication, or uridines will be replaced during replication, if at all.

Thus, inhibiting unwanted type I IFN production is challenging for SAM vaccines. It is documented that the ZIKV NS5 protein binds to and degrades human signal transducer and activator of transcription (STAT) 2 proteins downstream of type I IFN receptor signaling, inhibiting the expression of IFN-stimulated genes[56, 57]. In addition, it has also been suggested that the NS5 protein may inhibit type I IFN signaling through IRF3 and TBK1 pathways[58].

Consequently, ZIKV evades host defense mechanisms and can efficiently replicate in human cells, but not mouse cells. The characteristics of Z7, i.e. its high efficiency of replication, its low level of safety concerns, and its capability of type I IFN inhibition in humans, make it an ideal candidate for the SAM vaccine development.

### SUMMARY OF THE INVENTION

The present invention relates to the development of a self-amplifying mRNA vaccine using a self-amplifying backbone of a live-attenuated ZIKV virus (Z7) by inserting a GC-rich sequence (a 50 RNA-nucleotides (nt) hairpin) into the 5′ untranslated region (UTR) of a pre-epidemic virus. This same 50 RNA nt hairpin can be employed to attenuate other types of live viruses than just the ZIKV virus.

The present invention is characterized by being the first Zika virus-based self-amplifying mRNA (SAM) platform. The invention includes an attenuated virus. Since Z7 has been shown as a potent anti-Zika vaccine with little pathogenicity, it is also expected that this Z7-based SAM platform will be effective without raising any significant safety concerns. Further, incorporating a novel CMV-T7 dual promoter to generate the same SAM RNA by both in vitro T7 transcription and in vivo host RNA polymerase is a novel feature of the invention.

Also, incorporating T2A into the SAM platform to separate the antigen from the viral protein without the need for a protease is another advantageous feature of the present invention. Further, incorporating a signal peptide (SP) into the SAM platform to allow antigen secretion from the host cell provides additional advantages for the present invention.

The modular design of the platform, as shown in FIG. 12 and Example 1, allows for numerous different combinations among T2A, SP, antigens, and fluorescent/luciferase, enabling broad application and flexibility of the present invention.

In one embodiment, the ZIKV Cambodian strain, FSS13025 was employed as the virus. The resultant viral strain is attenuated in neurovirulence, immune antagonism, and mosquito infectivity compared with American Zika virus epidemic isolates.

The present invention demonstrates that Z7 replicates efficiently produce high titers, apparently without causing cytopathic effects (CPE) in Vero cells or losing the insert sequence, even after ten passages. Significantly, in one aspect, Z7 induces robust humoral and cellular immune responses that completely prevent viremia after a challenge with a high dose of the American epidemic ZIKV strain PRVABC59 infection in type I interferon (IFN) receptor A deficient (Ifnar1−/−) mice. Moreover, adoptive transfer of plasma collected from Z7 immunized mice protected Ifnar1−/− mice from ZIKV (strain PRVABC59) infection. These results demonstrate that Z7 can be used as a live-attenuated vaccine for viruses such as Zika viruses. In addition, the data presented herein also indicate that modifying the 5′ UTR is a strategy that can be used to develop other live-attenuated viruses such as, for example, flaviviruses and potentially develop vaccine candidates for other viruses and flaviviruses.

Z7, disclosed herein, possesses the following advantages for the development of safe and effective SAM mRNA vaccines:
(1) Z7 can infect target cells but doesn't cause, or causes very little, cell death, indicating that it is a safe SAM replicon; and
(2) Z7 can replicate very well and produce large amounts of viral RNA, suggesting that it will be effective in mRNA vaccine amplification.

In another aspect, the present invention relates to the novel use of flavivirus (FV) rather than commonly used alphavirus (AV)-based SAM to improve vaccine efficacy.

1. In one aspect, the invention relates to a self-amplifying mRNA vaccine, comprising:
a self-amplifying backbone of a live attenuated virus, Z7, having a hairpin loop insert, and wherein at least one or more structural genes of the live attenuated virus have been replaced with at least one target antigen-encoding nucleic acid sequence.

2. The vaccine of sentence 1, wherein the self-amplifying backbone comprises the first 50 amino acids of a capsid (C) protein and the last 30 amino acids of an envelope glycoprotein (E).

3. The vaccine of any one of sentences 1-2, wherein the hairpin loop insert comprises a nucleic acid sequence comprising 50 nucleotides.

4. The vaccine of any one of sentences 1-3, wherein the hairpin loop insert is an RNA sequence CGUUCCAACCACUGACUCGAAAGAGUCAGUGGUUGGAACGCGCAGGUGCC (SEQ ID NO: 10), in the 5′ untranslated region of the virus.

5. The vaccine of any one of sentences 1-4, wherein the hairpin loop insert comprises a paired stem region and an unpaired middle loop, wherein the stem region comprises greater than 50% cytosine-guanine nucleotide pairs, based on the total combined number of adenine-uracil nucleotide pairs and cytosine-guanine nucleotide pairs.

6. The vaccine of any one of sentences 1-5, further comprising an encapsulating component for delivery, selected from the group consisting of a lipid-nanoparticle and a polymer.

7. The vaccine of any one of sentences 1-6, wherein target antigen is derived from an RNA virus selected from the group consisting of an alphavirus, flavivirus, picornaviruses, coronaviruses, retroviruses, paramyxoviruses, rhabdoviruses, orthomyxoviruses, filoviruses, rotaviruses, orthopneumovirus, and arteriviruses.

8. The vaccine of sentence 7, wherein the RNA virus is an alphavirus selected from the group consisting of Chikungunya virus, Ross River Virus, Sindbis virus, Mayaro virus, Semliki Forest virus, Eastern equine encephalitis virus, Western equine encephalitis virus, Venezuelan equine encephalitis virus, and Aura virus.

9. The vaccine of sentence 7, wherein the RNA virus is a flavivirus, is selected from the group consisting of West Nile Virus, Dengue Virus, Tick-borne encephalitis virus, Yellow Fever Virus, and Zika Virus.

10. The vaccine of any one of sentences 1-9, wherein the target antigen is derived from a Chikungunya virus.

11. The vaccine of any one of sentences 1-10, wherein the live attenuated virus is Zika virus.

12. The vaccine of sentence 11, wherein the Zika virus is the Cambodian strain FSS13025.

13. The vaccine of any one of sentences 1-6, wherein the target antigen is a viral structural protein on an outer surface of a viral particle.

14. The vaccine of sentence 13, wherein the viral structural protein is selected from the group consisting of spike proteins and envelope proteins.

15. The vaccine of sentence 13, wherein the viral structural protein is a protein that interacts with host cell receptors to facilitate the viral particle gaining entry into the host cell.

16. The vaccine of any one of sentences 1-6, wherein the target antigen is a cancer specific antigen or a tumor specific antigen.

17. The vaccine of sentence 16, wherein the cancer or tumor specific antigen is located on an outer surface of a cancer or tumor cell.

18. The vaccine of sentence 16, wherein the tumor specific antigen is selected from the group consisting of CA-125, CA15-3, CA19-9, hCG, beta-hCG, PSA, Calcitonin, SCC, URLC10AFP, CD19, TRAC, TCRB, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, epithelial tumor antigen, mesothelin, IL-11Ra, PSCA, PRSS21, VEGFR1, VEGFR2, LewisY, CD24, PDGFR-beta, SSEA-4, CD20, folate receptor alpha, ERBB2 (Her2/neu), MUC-1, MUC-2, MUC16, EGFR, NCAM, prostase, PAP, ELF2M, Ephrin B2, IGF-I receptor, CAIX, LMP2, gp100, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, WT1HPV16 E7, HPV E6, E7, MAGE A1, ETV6-AML, sperm protein 17, XAGE1, Tie 2, MAD-CT-1, MAD-CT-2, Fos-related antigen 1, p53, p53 mutant, protein, surviving, telomerase, PCTA-1/Galectin 8, MelanA/MART1, Ras mutant, hTERT, sarcoma translocation breakpoints, gp100, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, MART-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3c, CD4, CD5, and CD7.

An example of the complete Z7-based SAM sequence for producing CHKV E2-linker-E1 antigen (the DNA sequence can be used directly for cellular delivery to launch SAM vaccine) is provided in SEQ ID NO: 19. The CHKV E2-linker-E1 antigen the DNA sequence can also be used as a template to prepare mRNA in vitro by T7 RNA polymerase, or it can inserted into suitable plasmid systems with low copy numbers and antibiotic markers:

### BRIEF DESCRIPTION OF THE DRAWINGS

FIGS. 1A-1B show the generation of mutant ZIKV by modifying the 5′ UTR. FIG. 1A shows the insertion site of the GC rich nucleotides in the 5′ UTR of the ZIKV genome 73. FIG. 1B shows the predicted secondary structure of Z1 (wildtype (WT), no insert), Z3 (18-nt insert), Z5 (38-nt insert), and Z7 (50-nt insert), as predicted by RNAFold.

FIGS. 2A-2E show the characterization of Z7 in vitro. The data in FIGS. 2A-2D are presented as mean±s.e.m.

FIG. 2A shows quantification of ZIKV copies of 5′ UTR modified (Z3, Z5, Z7) or unmodified (Z1) plasmids by qRT-PCR.

FIG. 2B shows quantification of ZIKV E in Z7 for several generations (G4 to G10) by qRT-PCR. Data were presented as the ratio of ZIKV E to Vero β-actin.

FIG. 2C shows the in vitro growth curves of Vero cell-generated Z1 and Z7 (G10). Vero cells were infected with 0.1 multiplicity of infection (MOI) of Z1 or Z7 (G10), and the virus titers in the supernatants were determined by focus formation assay (FFA).

FIG. 2D shows the in vitro growth curves of HEK-293 cell-generated Z1 and Z7 (G11). HEK-293 cells were infected with 0.01 MOI of Z1 or Z7 (G11), and the virus titers in the supernatants were determined by FFA.

FIG. 2E shows transmission electron microscopy (TEM) images of Z1 and Z7 (G9). The data for Figs.

FIGS. 3A-3E show that Z7 exhibited attenuated infectivity in vitro. FIG. 3A shows the cytopathic effect (CPE) of Z1 and Z7 on Vero cells. Vero cells were inoculated with Z1, Z7 (G11), or phosphate-buffered saline (PBS) as a control and incubated for 3 days. The cells were stained with LIVE/DEAD Cell Imaging Kit, which stained the live cells with fluorescein (FITC) as green and the dead cell nuclei with Texas-red as red. The images were taken at 10× magnification (scale bar=100 μm).

FIG. 3B shows an immunofluorescence analysis (IFA) for ZIKV E protein expression. Vero cells were infected with 0.1 MOI of Z1, Z7 (G11), or PBS as control and incubated for 3 days. The ZIKV E protein was probed with 4G2 antibody, followed by goat anti-mouse IgG conjugated with Alexa Fluor 488 (green). The cell nuclei were stained with 4′,6-diamidino-2-phenylindole (DAPI, blue). The images were taken at 20× magnification (scale bar=100 μm).

FIG. 3C shows immunostaining foci of Z1 and Z7. Vero cells were infected with 10 or 100 fluorescent focus units (FFU) of Z1, Z7 (G11), or PBS as control and incubated for 3 days (Z1) or 4 days (Z7). The cells were probed with 4G2 antibody and then goat anti-mouse IgG-HRP as a secondary antibody. The immuno-positive foci were developed with TrueBlue peroxidase substrate.

FIG. 3D shows plaque morphology of ZIKV (strain PRVABC59), Z1, and Z7. The Vero cell monolayer was infected with 100 μl of serially diluted ZIKV (strain PRVABC59), Z1 or Z7 (G11) and incubated for 5 days.

FIG. 3E shows plaque sizes of ZIKV (strain PRVABC59), Z1, and Z7 on day 5 post-immunization data were compared with a two-tailed Student's t-test and presented as mean f s.e.m. with ****p<0.0001.

FIGS. 4A-4E show that Z7 exhibited attenuated infectivity in Ifnar1^{-/-} mice. Four-week-old Ifnar1^{-/-} mice were inoculated with 1×10⁵ FFU of Z1, or Z7 (G10) through their footpads.

FIG. 4A shows the relative changes in body weight.

FIG. 4B shows survival curves. Mice were monitored daily for 21 days to determine survival.

FIG. 4C shows viremia of Z1 and Z7 infected mice. Mice were bled every alternate day from day 1 to day 11 post-immunization, and the viremia was determined by measuring the ZIKV genome copies (ZIKV E) by qRT-PCR.

FIG. 4D shows viral load in the liver and spleen tissues. Z1 or Z7 (G10)-infected mice were sacrificed on day 3 post-immunization to harvest the livers and spleens. The viral burden was measured by qRT-PCR and expressed as the ratio of the copy numbers of ZIKV E to mouse β-actin.

FIG. 4E shows viral RNA extracted from the viral stocks containing 1×10⁵ FFU of Z1 or Z7 (G10). The ZIKV E genome copies were quantified by qRT-PCR. Data were compared with a log-rank test (FIG. 4B), Mann-Whitney U tests (FIGS. 4C and 4E), a two-tailed Student's t-test (FIG. 4D) and presented as mean±s.e.m. with *p<0.05, **p<0.01, ***p<0.001, and ****p<0.0001.

FIGS. 5A-5D show Z7 induced robust IgG and T-cell responses in mice.

FIG. 5A shows the anti-ZIKV E IgG response of Z7 immunization. Four-week-old Ifnar1^{-/-} mice were immunized with 1×10⁵ FFU of Z7 (G10) via footpad. Anti-ZIKV E IgG was measured in plasma samples from D0 (pre-immunization) and day 24 (post-immunization) by ELISA.

FIG. 5B shows the results of plaque reduction neutralization tests (PRNT) of six 4-week-old Ifnar1−/− mice immunized with 2×10⁴ FFU of Z7 (G11) via footpad. On day 24 post-immunization, plasma samples were collected. The ZIKV neutralizing capacity of the plasma from each mouse was determined by PRNT.

FIGS. 5C and 5D show cellular immune responses of Z7 immunization. Seven-week-old Ifnar1^{-/-} mice were immunized with 1×10⁵ FFU of Z7 (G10) or PBS (control) via footpad. On day 8 post-immunization, splenocytes were collected and re-stimulated with Z1 ex vivo for 24 hours. The interferon gamma (IFN-7) producing (FIG. 5C) CD4+ and (FIG. 5D) CD8+ T cells were measured by flow cytometry. Data were compared with a two-tailed Student's t-test (FIG. 5A), Mann-Whitney U tests (FIGS. 5C and 5D) and presented as mean±s.e.m. with *p<0.05, and ****p<0.0001.

FIGS. 6A, 6B, and 6C show Z7 induced sterilizing immunity in Ifnar1−/− mice against an epidemic ZIKV strain infection. Four-week-old Ifnar1−/− mice were inoculated with 1×10⁵ FFU of Z7 (G10) or PBS as control via footpad. On day 42 post-immunization, the mice were challenged with 1×10⁵ PFU of ZIKV (strain PRVABC59) via footpad. (FIGS. 6A and 6B) Mice were bled on day 1 to day 3 post-challenge (p.c.), and viremia was measured by qRT-PCR on D1 to D3 p.c. (FIG. 6A) and plaque assay on day 3 p.c. samples (FIG. 6B).

FIG. 6C shows viral load in the liver and spleen tissues. Z1 or Z7-infected mice were sacrificed on day 3 post-immunization to harvest the liver and spleen. The viral load in the tissues was measured by qRT-PCR and expressed as the ratio of ZIKV E to mouse p-actin. Data were compared with Mann-Whitney U tests (FIGS. 6A and 6B), a two-tailed Student's t-test (FIG. 6C) and presented as mean±s.e.m. with *p<0.05, and **p<0.01.

FIGS. 7A-7D show Z7 that immunized plasma protects Ifnar1−/− mice from ZIKV infection. Five-week-old Ifnar1 mice were infused with 100 μl of plasma collected from Z7 (G10) immunized or control mice via retro-orbital injection. After 24 h, the mice were challenged with 1×10⁵ PFU of ZIKV (strain PRVAVC59) via footpad inoculation.

FIG. 7A shows relative changes in body weight.

FIG. 7B shows survival curves.

FIG. 7C shows viremia measured by qRT-PCR.

FIG. 7D shows a schematic diagram of an adoptive transfer study.

FIGS. 8A-8C show schematic illustrations of SAM replicons.

FIG. 8A shows a schematic of Z7- or VEEV based SAM replicons and Chikungunya Virus (CHIKV) E2-E1 protein-superfold (sf)GFP.

FIG. 8B shows Z7-based—E2-E1 replicon plasmid.

FIG. 8C shows VEEV-based—E2-E1 replicon plasmid.

FIG. 9 shows that HEK-293 cells transfected with Z7-based—E2-E1—replicon plasmids express more intensive GFP signals than those transfected with VEEV-based—E2-E1—replicons, as viewed under a confocal microscope.

FIGS. 10A-10D show that GFP-expressing HEK-293 cells transfected with Z7-based—E2-E1—replicon plasmids express more intensive GFP signals than those transfected with VEEV-based—E2-E1—replicons, as quantified by flow cytometry.

FIG. 10A shows the flow cytometry results for the transfection control.

FIG. 10B shows the flow cytometry results for the VEEV plasmid.

FIG. 10C shows the flow cytometry results for the Z7 plasmid.

FIG. 10D shows the GFP-expressing HEK-293 cells transfected with the Z7 plasmid, VEEV plasmid, and the transfection control.

FIG. 11 shows that HEK-293 cells transfected with Z7-based—E2-E1—replicon plasmids expressed approximately 10 times more CHIKV E1 mRNA than those transfected with VEEV-based—E2-E1—replicons.

FIG. 12 shows a schematic of the self-amplifying mRNA System for generating anticancer and antiviral vaccines in accordance with the present invention.

### DETAILED DESCRIPTION

### Definitions

As used herein, the phrases, “hairpin RNA structure,” “hairpin loop,” “hairpin insertion,” “hairpin insert,” and other variants, refer to a segment of a single-stranded RNA molecule, or mRNA, folding back on itself and creating a double-stranded region due to a complementary base pairing within the same RNA sequence. The structure resembles a hairpin, with a stem formed by base pairs and an unpaired loop at the end.

As used herein, the phrases, “CG rich hairpin,” “C-G rich hairpin,” and “cytosine-guanine rich hairpin” refer to the stem region of a hairpin. In a strand of RNA, there are four types of nucleotides, i.e. adenine (A), uracil (U), cytosine (C), and guanine (G). A-U and C-G can form pairs in the stem region of a hairpin. Thus, a CG rich hairpin means the stem region of the hairpin includes greater than 50% C-G pairs based on the total number of C-G pairs and A-U pairs. A higher concentration of C-G pairs is advantageous since these pairs have a stronger binding strength than A-U pairs, and thus, corresponds to enhanced stability of the hairpin.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g. in light of history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The present invention relates to a self-amplifying (SAM) mRNA based on an attenuated virus such as a flavivirus, e.g. Zika virus, which is superior to the commonly used alphavirus-based SAM vaccine platforms. The present invention relates to using established cloning techniques and a 50 nt hairpin insert to construct novel virus-based SAM replicons.

ZIKV has demonstrated high replication efficiency in humans, both in vitro and in vivo, and thus, preparing a vaccine system based on the ZIKV genome is advantageous.

For example, the attenuated flavivirus may be based on a Zika virus (Z7) developed using Zika Cambodian strain (Z1). The attenuated Zika virus, Z7, was generated by inserting a GC rich 50 nucleotide DNA sequence at the 5′ UTR of Z1.

The flavivirus genome has a 5′ AG sequence that may be important for viral genome replication, potentially leading to a lower replication efficiency with a modified 5′ end. While an additional G has to be added to construct flavivirus replicons for in vitro RNA preparation [48], previously developed in vitro transcription (IVR) systems that initiate RNA with AG exist, thereby producing a genuine 5′ end of the viral replicon, which leads to high mRNA amplification efficiency and potent immune responses.

The live attenuated Zika virus comprises structural genes (the capsid protein, membrane protein, and envelope glycoprotein) and non-structural genes (NS1-NS5). To construct the self-amplifying mRNA vaccine of the present invention, at least one or more of the structural genes are replaced with at least one target antigen-encoding nucleic acid sequence. Preferably, the backbone live attenuated Zika virus comprises at least the first 50 amino acids of a capsid (C) protein and at least the last 30 amino acids of an envelope glycoprotein (E), and is optionally, devoid of the membrane protein (prM).

Although viral SAM replicons do not form viral particles, pathogenic ZIKV genome-based replicons may still cause safety concerns when used in humans, and a live-attenuated ZIKV is preferable for this purpose. For example, the live-attenuated ZIKV strain employed by the present invention, Z7, was prepared by inserting 50 RNA nucleotides (nt) into the 5′ untranslated region (UTR) of a pre-epidemic ZIKV Cambodian strain, FSS13025. This strain was used to develop live-attenuated ZIKV because it is attenuated in neurovirulence, immune antagonism, and mosquito infectivity compared to American epidemic isolates[49]. The examples set forth herein demonstrate that Z7 replicates efficiently and produces high titers without causing apparent cytopathic effects (CPE) in Vero cells or losing the insert sequence, even after ten passages. In addition, Z7 injection did not cause any sign of sickness in Ifnar1−/− mice, although low levels of viral load in blood and the peripheral tissues can be detected in Z7-infected mice.

These results confirmed that Z7 has an attenuated infectivity compared to its wild-type (WT) parental strain. Moreover, two types of mutations in the 1417 position, S1417A (majority) or S1417T (minority), were identified in the NS2B protein of Z7. The NS2B protein pairs with NS3 protease to cleave viral proteins and facilitate NS3 proteolytic activity, contributing to the host cell apoptosis and neuropathogenesis [50, 51]. It has been reported that mutations in NS2B significantly alter the interaction between NS2B and NS3, followed by a decrease in the NS3 protease activity and ZIKV replication [52]. These results confirm that Z7 possesses desirable characteristics of SAM replicons in high efficiency of replication in vitro and in vivo and low safety concerns.

Flaviviruses have a linear, positive-sense, single-stranded RNA genome, which is comprised of a 5′ untranslated region (UTR), an open reading frame that translates into a polyprotein, and a 3′ UTR²¹⁻²³. The polyprotein is cleaved by the cellular and viral proteases into 3 structural proteins (capsid [C], pre-membrane [PrM/M], and envelope [E]) and 7 non-structural proteins (NS1, NS2A, NS2B, NS3, NS4A, NS4B, and NS5)^{21,24}.

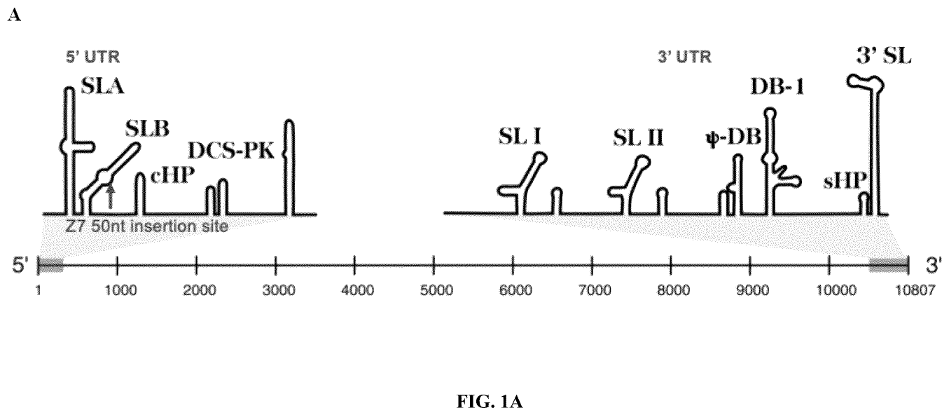

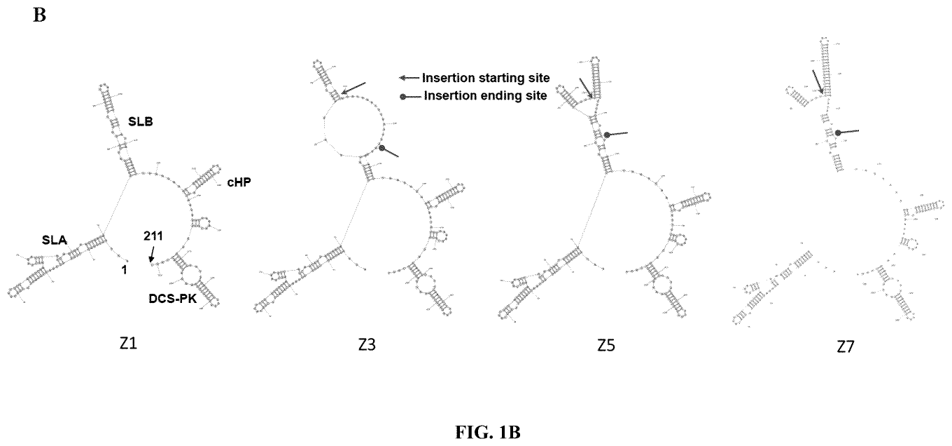

In ZIKV, the 5′ UTR is 106 nucleotides in length, and the 3′ UTR is 428 nucleotides in length⁵. The ZIKV 5′ UTR consists of an m7GpppAmpN1 cap structure and the conserved stem loops A and B (SLA and SLB, FIG. 1A)^{26,27}.

The cap structure in flavivirus genomes is important for cap-dependent translation and protection from cellular 5′-3′ exonucleases^{26,28}. While SLA serves as a promoter for the viral RNA-dependent RNA polymerase (RdRp) NS5, SLB facilitates replication by cyclizing the RNA genome via the formation of a 5′-3′ complementary structure with the 3′ UTR^{26,29,30}. A recent study also found that the ZIKV 5′ UTR pairs with the E protein coding region to form a 5′-E complementary structure, which may play an important role in viral replication or translation regulation. Other conserved RNA structural elements, such as the C coding region hairpin (cHP) and the downstream of 5′ cyclization sequence pseudoknot (DCS-PK), were also identified at the 5′ end of the ZIKV genome²⁶.

Although the 5′ UTR sequences are variable among flaviviruses, the RNA structure is mostly conserved and is essential for genome cyclization, viral RNA synthesis, translation, and viral fitness. The 3′ UTR of ZIKV folds into three domains which are highly structured with regions conserved among flaviviruses (FIG. 1A)^{25,26}. Interacting with the 5′ UTR, the 3′ UTR plays a critical role for viral replication²⁶.

In eukaryotic translation, ribosomes scan, beginning from the methylated cap, for an AUG start codon and stall at areas with stable secondary structures, such as stem-loops since the ribosomes have to “melt” these structures to continue down the RNA strand³¹. A previous report showed that by adding various lengths of stem loops in eukaryotic cells, translation was slowed due to the increased length of the GC-rich structures³².

While the 5′ UTR insertion method has been used to attenuate translation in different eukaryotic systems^{33,34}, it has apparently not been applied to viral research. Without being bound by theory, the present inventors believe that modification of the 5′ UTR of the ZIKV genome by inserting a GC-rich sequence should introduce an additional RNA structure that might attenuate circularization and slow down the rate of viral RNA replication and protein translation, thus creating an attenuated virus.

The Z7 replicon platform may be modified for the development of vaccines against other viral pathogens and cancers. For example, the self-amplifying backbone comprises a live attenuated virus having a hairpin loop insert, wherein at least one or more structural genes of the live attenuated virus are replaced with at least one target antigen-encoding nucleic acid sequence. The target antigen may be derived from a viral pathogen or a cancer specific antigen.

For example, the Z7 replicon may be used for the development of SAM vaccines against viral pathogens, including but not limited to the viruses in the family of Togaviridae, Coronaviridae, Flaviviridae, Piconaviridae, Paramyxoviridae, Rhabdoviridae, Filoviridae, Bunyaviridae, Orthomyxoviridae, Papillomaviridae, Herpesviridae, Poxviridae, and Retroviridae.

The Z7 replicon may also be used for the development of SAM vaccines against cancers, including but not limited to breast cancer, prostate cancer, and pancreatic cancer.

Suitable target antigens may be derived from an RNA virus selected from alphavirus, flavivirus, picornaviruses, coronaviruses, retroviruses, paramyxoviruses, rhabdoviruses, orthomyxoviruses, filoviruses, rotaviruses, orthopneumovirus, togaviruses, and arteriviruses.

Target antigens derived from an alphaviruses may include Chikungunya virus, Ross River Virus, Sindbis virus, Mayaro virus, Semliki Forest virus, Eastern equine encephalitis virus, Western equine encephalitis virus, Venezuelan equine encephalitis virus, Aura virus, and any other type of flavivirus.

Target antigens derived from a flavivirus may include West Nile Virus, Dengue Virus, Tick-borne encephalitis virus, Yellow Fever Virus, Zika Virus, and any other type of flavivirus.

Target antigens derived from piconaviruses may include Enterovirus, foot-and-mouth disease virus, Hepatitis A virus, Parechovirus, Aichivirus A, Tremovirus, and any other type of piconavirus.

Target antigens derived from coronaviruses may include human coronavirus OC43 (HCoV-OC43), human coronavirus HKU1 (HCoV-HKU1), human coronavirus 229E (HCoV-229E), human coronavirus NL63 (HCoV-NL63), Severe acute respiratory syndrome coronavirus (SARS-CoV), Middle East Respiratory syndrome-related coronavirus (MERS-CoV), Severe Acute Respiratory Syndrome Coronavirus 2 (SARS-CoV-2).

Target antigens derived from Retroviruses may include viruses from each of the subfamilies Oncoviruses and Spumaviruses. Oncoviruses may include Alpharetrovirus, Betaretrovirus, Gammaretrovirus, Deltaretrovirus, Epsilonretrovirus, Lentivirus, and Spumaviruses may include Bovispumavirus, Equispumavirus, Felispumavirus, Prosimiispumavrius, and Simiispumavirus.

Target antigens derived from paramyxoviruses may include viruses from the subfamilies Avulavirinae, Metaparamyxovirinae, Orthoparamyxovirinae, and Rubulavirnae.

Target antigens derived from Rhabdoviruses may include Lyssavirus, Vesiculovirus, Ephemerovirus, Novirhabdovirus, and Tibovirus.

Target antigens derived from Orthomyxoviruses may include Alphainfluenzavirus, Betainfluenzavirus, Gammainfluenzavirus, Deltainfluenzavirus, Isavirus, Quaranjavirus, and Thogotovirus.

Target antigens derived from Filoviruses may include Cuevavirus, Dianlovirus, Ebolavirus, Marburgvirus, Striavirus, and Thamnovirus.

Target antigens derived from Rotaviruses may include Rotaviruses A, Rotaviruses B, Rotaviruses C, Rotaviruses D, Rotaviruses E, Rotaviruses F, Rotaviruses G, Rotaviruses H, Rotaviruses I, and Rotaviruses J.

Target antigens derived from Orthopneumovirus may include human respiratory syncytial virus (hRSV) and human orthopneumovirus.

Suitable target antigens may be cancer specific antigen or tumor specific antigen. For example, the target antigen may include cancer specific antigens or tumor specific antigens for treating breast cancer, bladder cancer, cervical cancer, colorectal cancer, leukemia, melanoma, non-small lung cell cancer, ovarian cancer, pancreatic cancer, prostate cancer, testicular cancer, thyroid cancer, Hodgkin lymphoma, and other cancers.

For example, the cancer specific antigen or tumor specific antigens may include an antigen selected from CA-125, CA15-3, CA19-9, hCG, beta-hCG, PSA, Calcitonin, SCC, URLC10AFP, CD19, TRAC, TCRB, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, epithelial tumor antigen, mesothelin, IL-11Ra, PSCA, PRSS21, VEGFR1, VEGFR2, LewisY, CD24, PDGFR-beta, SSEA-4, CD20, folate receptor alpha, ERBB2 (Her2/neu), MUC-1, MUC-2, MUC16, EGFR, NCAM, prostase, PAP, ELF2M, Ephrin B2, IGF-I receptor, CAIX, LMP2, gp100, bcr-abl, tyrosinase, EphA2, fucosyl GM1, sLe, GM3, TGS5, HNWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, WTIHPV16 E7, HPV E6, E7, MAGE A1, ETV6-AML, sperm protein 17, XAGE1, Tie 2, MAD-CT-1, MAD-CT-2, Fos-related antigen 1, p53, p53 mutant, protein, surviving, telomerase, PCTA-1/Galectin 8, MelanA/MART1, Ras mutant, hTERT, sarcoma translocation breakpoints, gp100, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, MART-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3c, CD4, CD5, and CD7.

### Generation of the Live-Attenuated ZIKV Strain (Z7)

To test if a modification of the 5′ UTR affects ZIKV infectivity, 18, 38, or 50 nucleotide GC-rich DNA sequences were inserted (Table 1) at the end of the stem loop B (SLB) region and before the start codon (ATG), or between the 5′ UTR and the capsid gene in the plasmids containing the ZIKV genome (Cambodian strain FSS13025) (FIG. 1A), respectively, by known cloning methods³⁵. The ZIKV Cambodian strain was selected as the cloning backbone because it is attenuated in neurovirulence, immune antagonism, and mosquito infectivity compared with the American epidemic isolates^{1,2,36}. The predicted 5′ UTR ZIKV RNA structures showed that the 38 and 50-nucleotide inserts resulted in an additional hairpin structure in the SLB, while the SLA, cHP, and DCS-PK structures remained intact (FIG. 1B, Z5 and Z7 respectively).

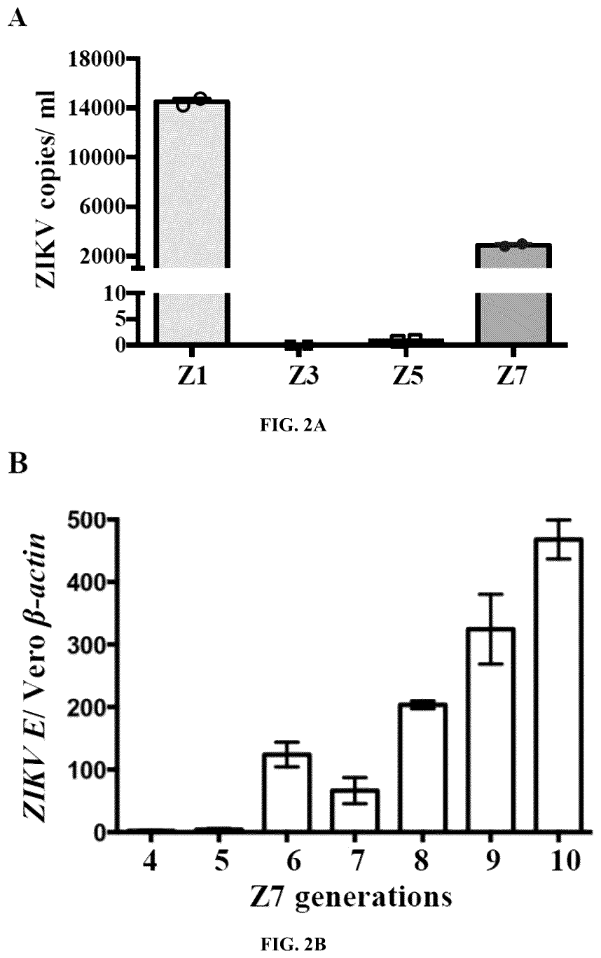

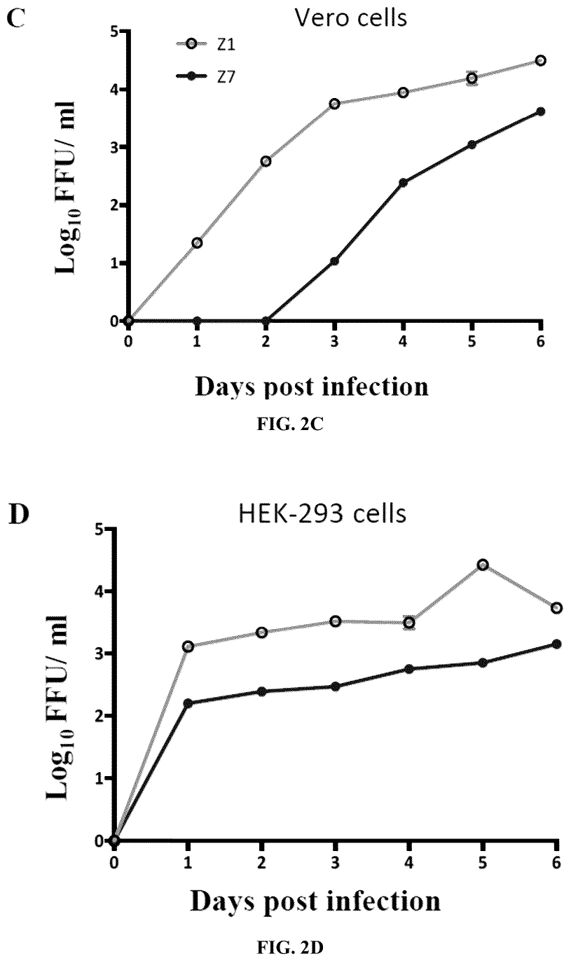

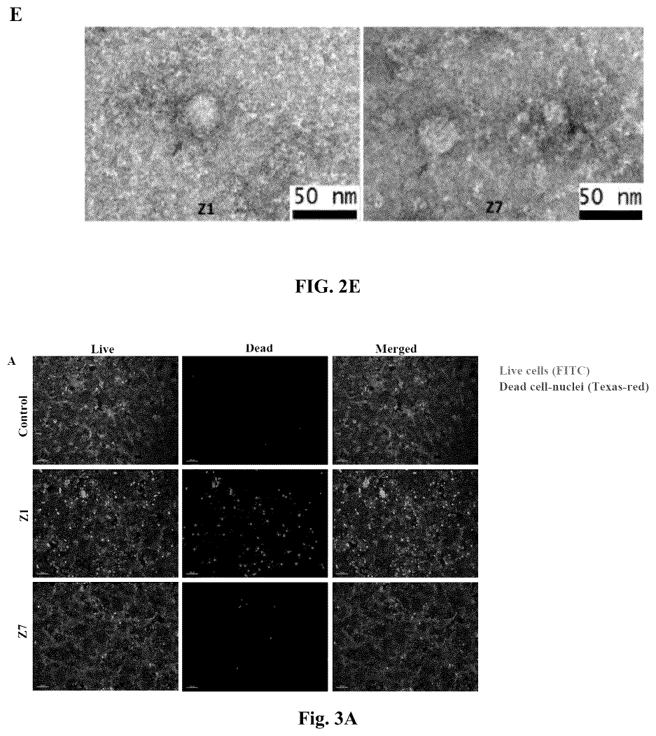

In contrast, instead of forming a new hairpin structure, the insertion of an 18-nucleotide insert resulted in a large loop in the SLB hairpin structure (FIG. 1B, Z3). These modified and unmodified control plasmids were transfected into Vero cells. The rescued ZIKVs (Z1=WT [no insert]; Z3=18 nucleotide insert; Z5=38 nucleotide insert; and Z7=50 nucleotide insert) in the cell culture media were collected on day 3 post-transfection. The media containing the first generation (G1) of ZIKVs were used to infect fresh Vero cells to evaluate the viral genome replication using the quantitative reverse transcription polymerase chain reaction (qRT-PCR). The qRT-PCR results indicated that insertion of 18-nucleotides (Z3) could not generate live viruses (FIG. 2A). The 38-nucleotide insertion (Z5) resulted in low but detectable ZIKVE copies, while the 50-nucleotide insertion (Z7) produced a much higher level of ZIKVE copies than Z5 (FIG. 2A).

TABLE 1
The Inserted RNA Sequences

| | Z1 | | | |
|---|---|---|---|---|
| Name | (WT) | Z3 | Z5 | Z7 |
| Inserted | None | CGUACGAG | CGACCACGG | SEQ ID NO: 10 |
| RNA | | CGCAGGUGC | CCGGAAACG | |
| Sequence | | (SEQ ID | GCCGUGGUCG | |
| | | NO: 26) | CGCAGGUGCC | |
| | | | (SEQ ID | |
| | | | NO: 25) | |

Z7 was continuously passed in Vero cells (an approved cell line for vaccine production³⁷) for 11 generations (5 days/generation). Titers of Z7 gradually increased through the continuous passaging in Vero cells, indicating that Z7 may have adapted fitness (FIG. 2B). Next, the growth kinetics of Z7 and Z1 were determined by a focus forming assay (FFA) in both Vero and Human Embryonic Kidney (HEK) 293 cells. Compared to Z1 (WT, Cambodian strain FSS13025), Z7 was able to grow efficiently in both cell lines, albeit with slightly lower growth rates (FIGS. 2C and 2D). To visualize these plasmid-generated ZIKV particles, Z1 and Z7 viral stocks were examined under a Transmission Electron Microscope (TEM) with negative staining. The TEM images confirmed that Z1 and Z7 exhibited the typical “golf ball” appearance with a size of approximately 40 nm of ZIKV 38 (FIG. 2E).

To determine if the 50-nucleotide insert remained stable during passaging, the whole viral genomes of Z7 (G8 to 10) and Z1 were sequenced by next generation sequencing, which confirmed the intact insert without mutation, indicating that the insert was stable in the 5′ UTR region. Interestingly, two mutations, S1417A (primary) and S1417T (secondary), in NS2B protein in Z7 (G8 to G10, data not shown) were discovered indicating Z7 might adopt a fitness mutation through passaging. Collectively, these results demonstrate that Z1 and Z7 were successfully generated with a reverse genetic approach, and that the 50-nucleotide insert in Z7 was stable for at least 10 generations.

### Z7 Exhibited Attenuated Infectivity In Vitro and in Ifnar1−/− Mice

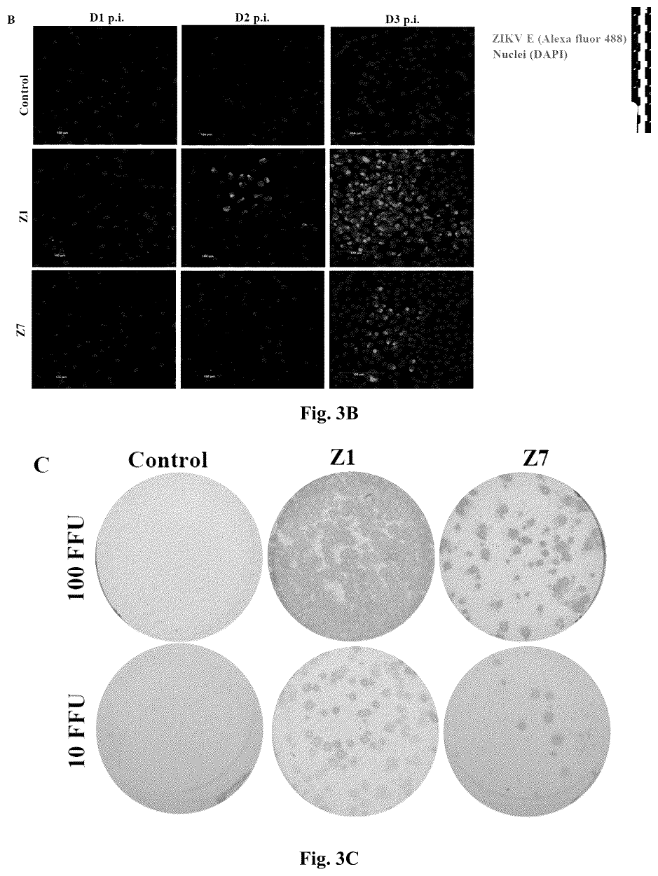

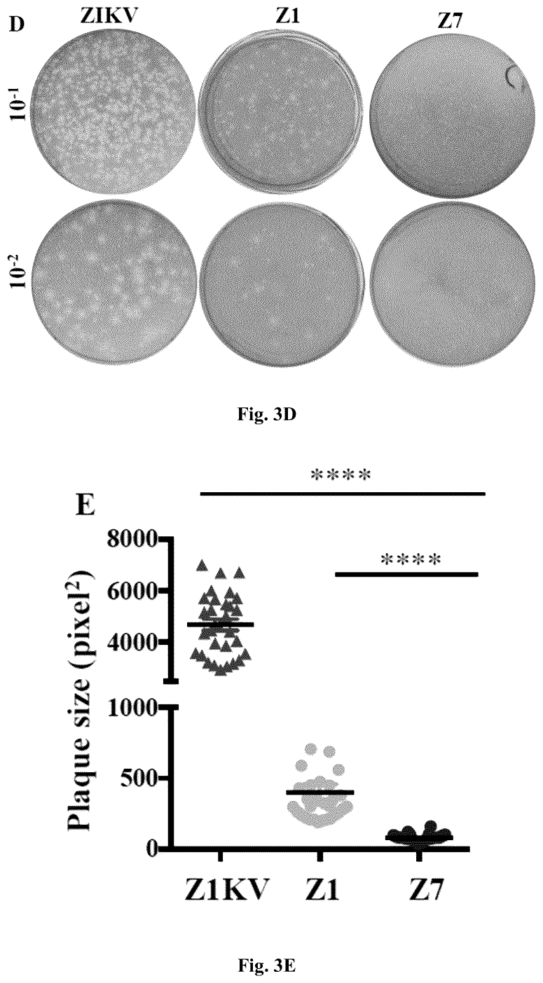

To determine if Z7 has attenuated pathogenicity compared to Z1, a cytopathic effect (CPE) assay, an immunofluorescence assay (IFA), focus forming assay (FFA), and a plaque-forming assay were performed. For the CPE assay, Vero cells were inoculated with 0.1 MOI of Z1, Z7 (G11), or PBS as control and the CPE on the cells was examined under a Leica M165 FC microscope. The results showed that Z1 caused apparent CPE effects in Vero cells starting on day 3 post infection (post-immunization) by killing more cells than Z7 (FIG. 3A), while Z7 did not cause noticeable CPE for 4 days compared to the control (day 4 post-immunization, data not shown).

To determine if Z7 is less infective in vitro, the expression of ZIKV E protein in Vero cells was measured by IFA, in which Vero cells were infected with 0.1 MOI of Z1, Z7 (11G), or PBS as control and incubated for 3 days. The confocal images showed that ZIKV E protein was detected in Z1-infected cells starting from day 2 post-immunization, while Z7-infected cells started to produce a lower level of the protein from day 3 compared with Z1-infected cells (FIG. 3B). Consistently, the FFA results also showed that Z7 took a longer time (4 days) than Z1 (3 days) to develop the ZIKV E positive immunofoci (FIG. 3C). In addition, in the plaque-forming assay, Z7 took a prolonged time (5 days) compared to Z1 and ZIKV (strain PRVABC59, 4 days) to develop visible plaques. Moreover, Z7 developed smaller sized plaques than Z1 and ZIKV (epidemic strain PRVABC59) on day 5 post-immunization (FIGS. 3D and 3E). These results collectively demonstrate that Z7 exhibited attenuated pathogenicity in vitro compared to Z1.

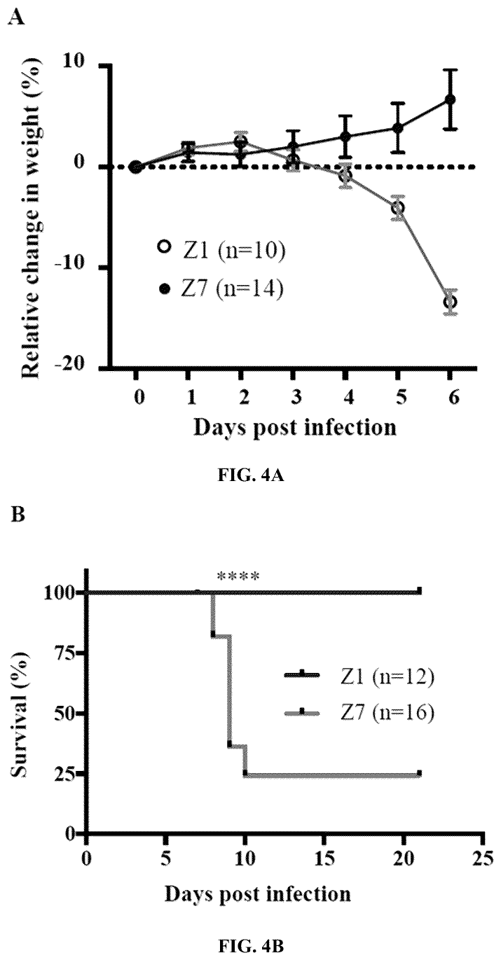

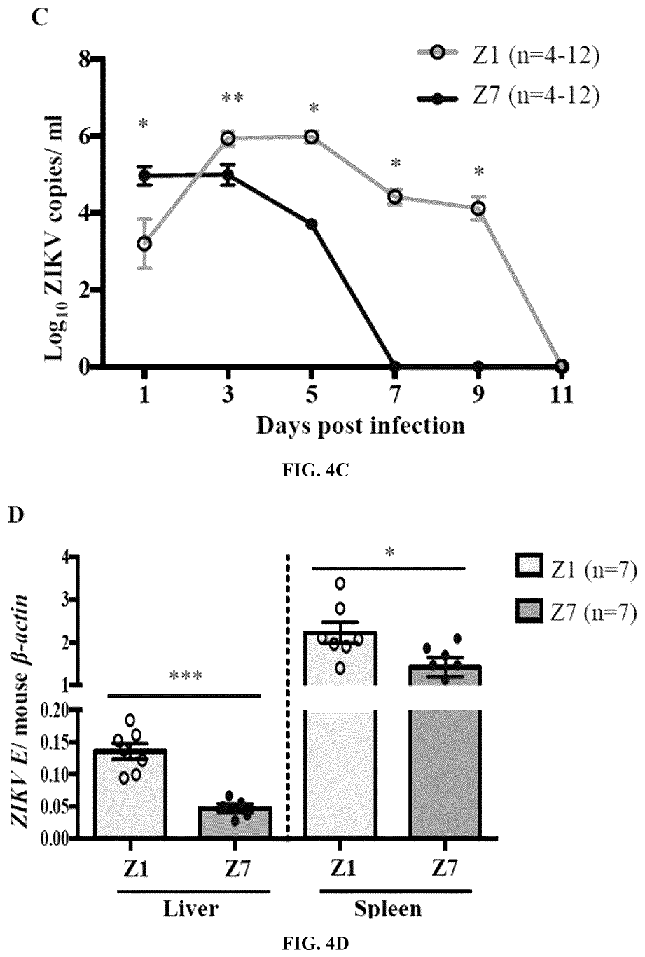

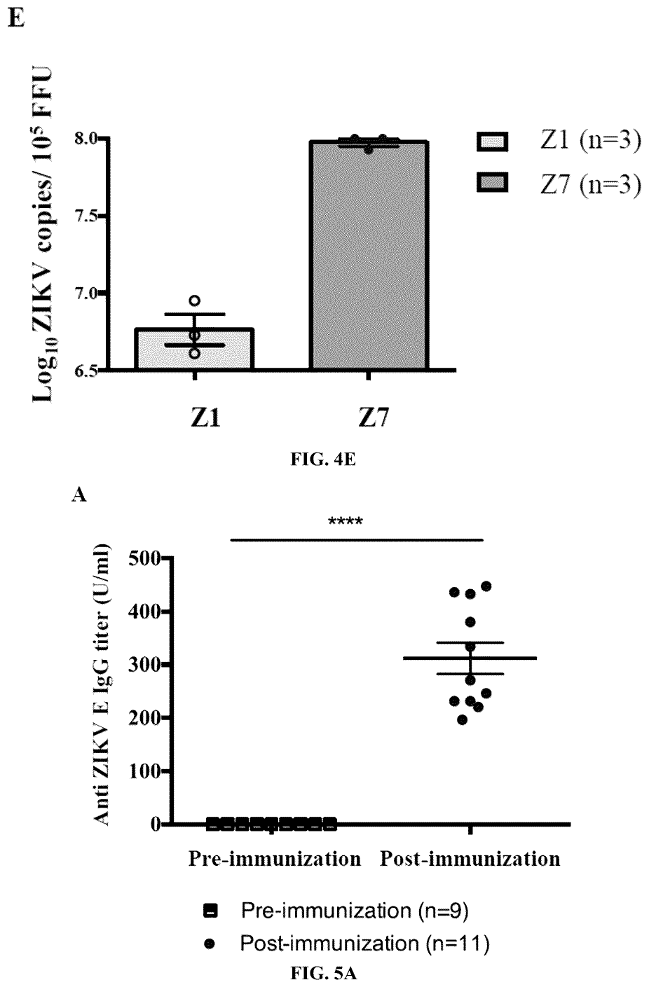

Z7 caused asymptomatic infection in ifnar1−/− mice To evaluate the pathogenicity of Z7 in a mouse model, a 4-week-old, sex-matched type I interferon (IFN) receptor A deficient (Ifnar1−/−) mice (in C57BL/6J background) were infected with 1×10⁵ focus-forming units (FFU) of Z7 (G10) or Z1 via footpad inoculation, which partially mimics mosquito transmission³⁹⁻⁴³. After infection, Z1-infected control mice began to lose body weight from day 4 post-immunization (FIG. 4A), death occurred starting from day 8 post-immunization, and 68% of the mice died by the end of the experiment (day 21 post-immunization, FIG. 4B). On the contrary, Z7 infected mice did not show any signs of sickness but instead continuously gained body weight, and 100% of the Z7 infected mice survived until the end of the experiment (FIGS. 4A and 4B).

To further evaluate the replication of Z7 in mice, separate groups of Ifnar1−/− mice were infected with Z1 or Z7 as above and the ZIKVE gene replication was measured in the blood, spleen, and liver by qRT-PCR. Consistent with the body weight and the survival results, Z7-infected mice generated a significantly lower viremia than Z1-infected animals from days 3 to 9 post-immunization, and the ZIKVE gene in Z7-infected blood samples became undetectable or below the pre-set qRT-PCR detection limit (Cq=38) after day 7 post-immunization (FIG. 4C). Similarly, Z7-infected mice also had reduced viral loads in the liver and spleen tissues compared to Z1-infected mice (FIG. 4D). Also, significantly more ZIKV genome copies were detected in the blood of the Z7-infected mice than in the blood of the Z1-infected mice on day 1 post-immunization (FIG. 4C). This may be an indication that there might be more non-infectious Z7 viral particles than Z1 in the inoculums. Thus, both in vitro and in vivo results strongly suggest that Z7 is a safe live-attenuated candidate for SAM vaccine development. To test if there might be more non-infectious Z7 viral particles than Z1 in the inoculums, 1×10⁵ FFU of Z1 and Z7 viral stocks were pretreated with RNase to remove any cellular and viral RNA that is free of the viral particles, then viral RNA was extracted from the viral nucleocapsids. ZIKV genome copies were quantified by measuring ZIKVE with qRT-PCR. The results showed 15-fold more ZIKV genome RNA copies in Z7 samples than Z1 samples within 1×10⁵ FFU of the viral stock samples (FIG. 4E). These results further indicated that Z7 possessed weakened infectivity and may produce a large amount of non-infectious viral particles, which could serve as a source of immunogens to induce anti-ZIKV immunity without causing disease. In summary, both in vitro and in vivo results strongly supported the conclusion that Z7 exhibited attenuated pathogenicity compared to its parental WT strain Z1.

### Z7 Induced Robust Humoral and Cellular Immune Responses in Ifnar1−/− Mice

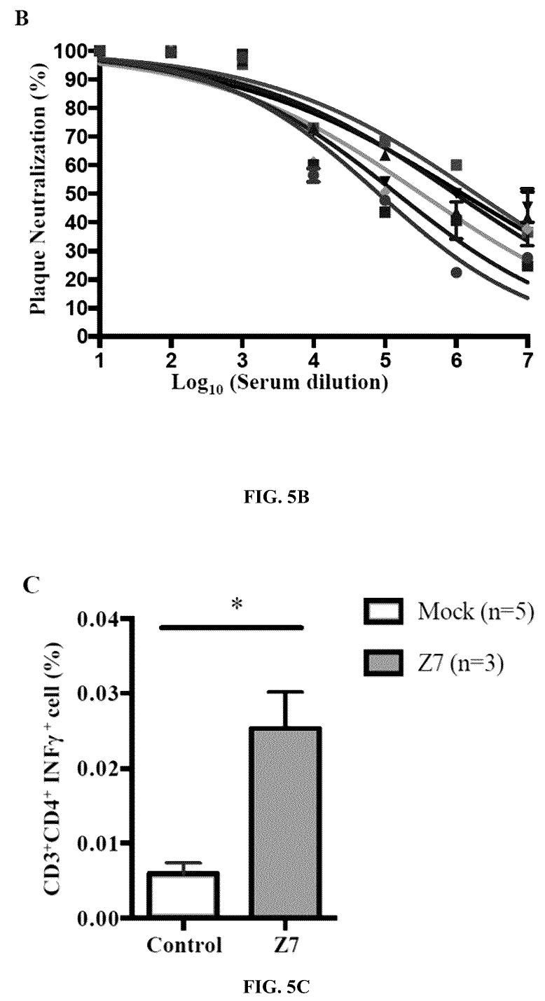

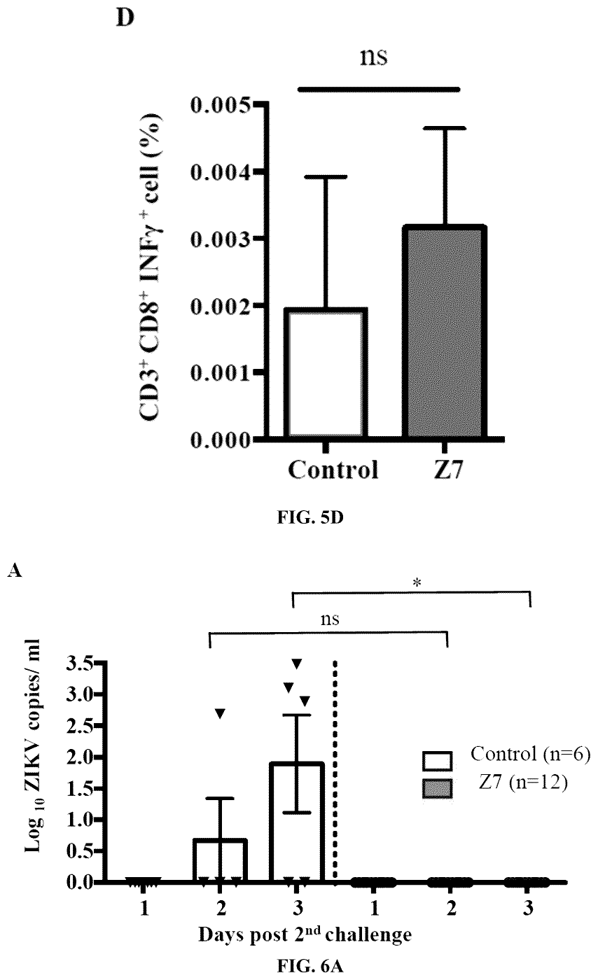

Since Z7 was generated by modifying the 5′ UTR of a non-epidemic ZIKV strain (FSS13025), and all of the structural and non-structural genes remained unchanged, it was hypothesized that Z7 could induce robust humoral and cellular immune responses. To test this, 4-week-old, sex-matched Ifnar1−/− mice were inoculated with 1×10⁵ FFU of Z7 via footpad. The blood samples were collected on day 0 (pre-immunization) and day 24 post-immunization. The levels of anti-ZIKV E IgG in the plasma were measured by enzyme-linked immunoassay (ELISA). The ELISA results indicated that Z7 induced a high titer of anti-ZIKV E IgG (mean value=311.7 U/ml) on day 24 post-immunization (FIG. 5A).

To measure if Z7-induced antibodies could efficiently neutralize the epidemic ZIKV (strain PRVABC59) in vitro, a plaque reduction neutralization test (PRNT) was performed. The range of the PRNT50 value of Z7-induced neutralizing antibody was between 104.9 to 106.2 (FIG. 5B). These results further support the conclusion that Z7 immunization induces robust anti-ZIKV neutralizing antibody responses. To measure the cellular immune responses, 7-week-old Ifnar1−/− mice were immunized with 1×10⁵ FFU of Z7 or PBS as control via footpad and the spleens were collected on day 8 post-immunization The splenocytes were re-stimulated with 0.1 MOI of Z1 ex vivo for 24 hours (h) to induce ZIKV-specific T cell responses. IFN-γ-producing CD4+ and CD8+ T cells were measured using flow cytometry. The results showed that the Z7-immunized mice induced an approximately 3-fold higher IFN-γ response in CD4+ T cells than the control group (FIG. 5C). However, there was no difference in IFN-γ producing CD8+ T cells between the Z7-immunized group and the control group (FIG. 5D). Thus, these results indicated that Z7 immunization can induce strong humoral and cellular immune responses in Ifnar1−/− mice.

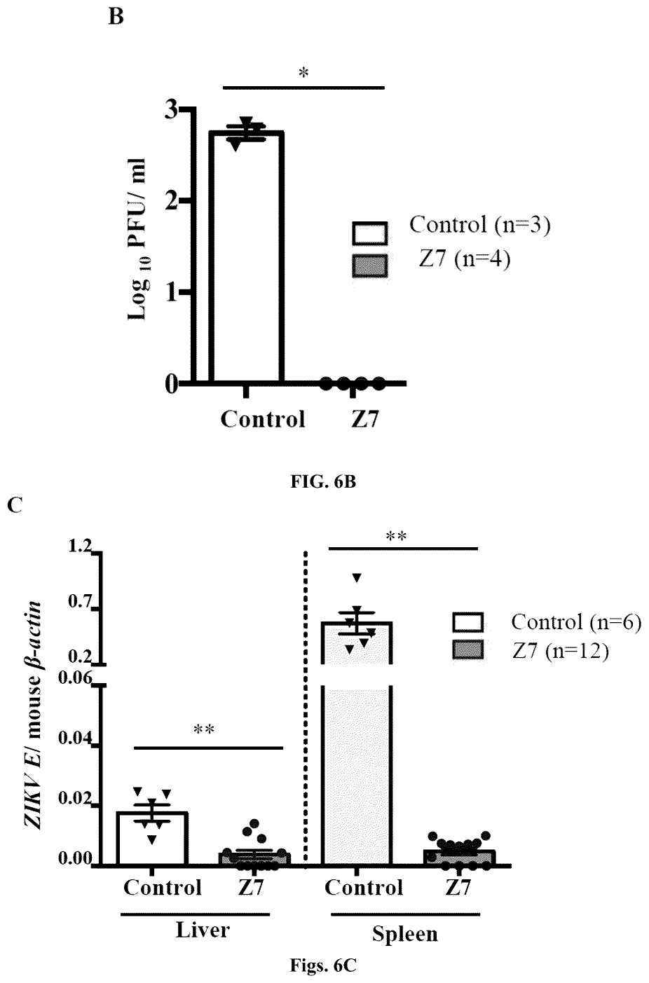

Z7 induced sterilizing immunity against ZIKV infection in Ifnar1−/− mice To evaluate if Z7-induced immunity can protect mice from epidemic ZIKV infection, 4-week-old Ifnar1−/− mice were immunized with either 1×10⁵ FFU of Z7 (G10) or PBS as control via footpad inoculation. On day 42 post-immunization, these mice were challenged through a footpad with 1×10⁵ plaque forming units (PFU) of ZIKV (strain PRVABC59). The blood samples were collected on days 1 to 3 post-challenge (p.c.) to measure the viremia by quantifying ZIKVE gene copies by qRT-PCR. There were no detectable ZIKVE genes in the blood samples of Z7-immunized mice at any time point, whereas ZIKVE genes were detected in the samples of the control group on days 2 and 3 p.c. (FIG. 6A). To confirm the qRT-PCR results, the infectious viral particles in the blood samples collected on day 3 p.c. were measured by plaque-forming assay. Similar to qRT-PCR, no plaque was developed in the blood samples of the Z7-immunized mice (FIG. 6B). To measure the viral load in the peripheral tissue after the challenge with the epidemic ZIKV infection, some mice were sacrificed on day 3 p.c. and the livers and spleens were collected to measure the viral load by qRT-PCR. Consistent with the viremia, the ZIKVE genes in the tissue samples of Z7-immunized mice were either not detectable or were below the set qRT-PCR detection limit (Cq=38). The control mice had significantly more ZIKVE genes in both their liver and spleen samples (FIG. 6C). Thus, these results suggest that a single dose of Z7 immunization can induce sterilizing immunity to protect mice against the epidemic ZIKV strain infection in the Ifnar1−/− mouse model.

Adoptive Transfer of Z7-Immunized Plasma Protected Ifnar1−/− Mice from ZIKV Infection

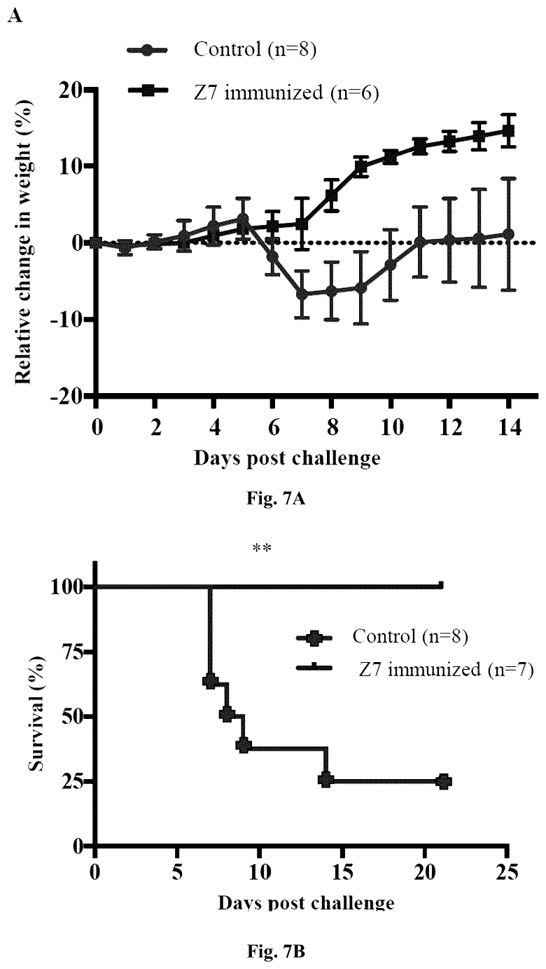

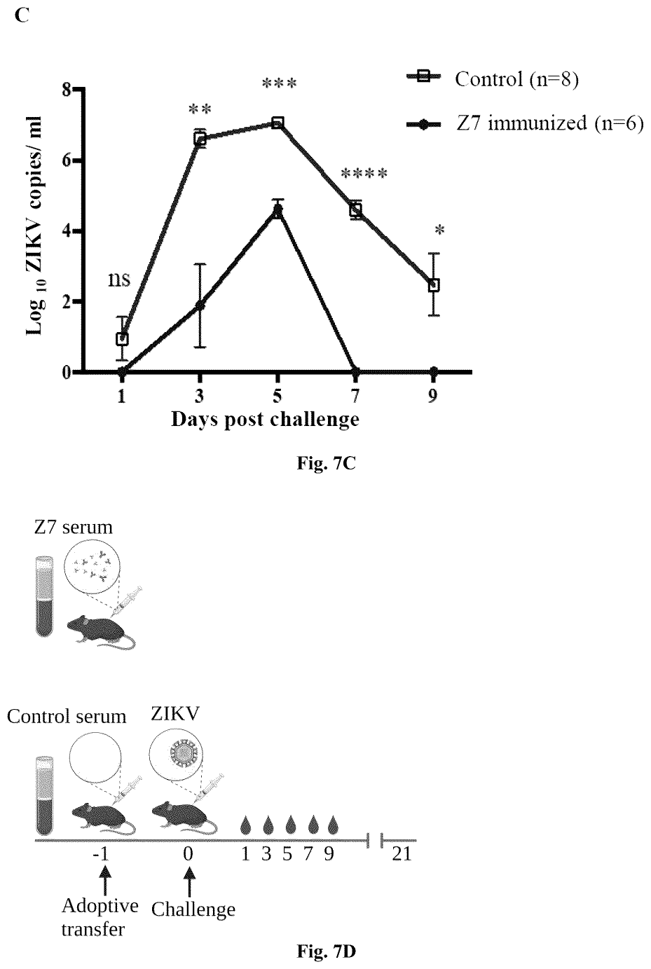

To evaluate if the plasma collected from the Z7-immunized mice protects against ZIKV infection, 100 μl of plasma collected from either Z7-immunized mice or mock-infected mice as control were adoptively transferred via retro-orbital injection in 5-week-old Ifnar1−/− mice. The next day, both groups were challenged with 1×10⁵ PFU of ZIKV (strain PRVABC59) via footpad and the body weight changes up to day 15 post-immunization were monitored. The control group started to lose weight from day 5 post-immunization until day 7 post-immunization followed by gaining weight from day 8 post-immunization until the end of the experiment (FIG. 7A). On the contrary, the mice which received the Z7-immunized plasma did not show any signs of sickness and gained body weight continuously.

To measure survival, 100 μl of Z7-immunized plasma or PBS as control were adoptively transferred via retro-orbital injection in 4-week-old Ifnar1−/− mice and these mice were challenged with 1×10⁵ PFU of ZIKV (strain PRVABC59) via footpad. The control group started to die from day 7 post-immunization and 75% of the mice died before the end of the experiment (day 21 post-immunization). In contrast, all of the Z7 plasma-infused animals survived (FIG. 7B).

To characterize the protective effects of Z7-immunized plasma, a separate adoptive transfer study was performed in a group of 5-week-old Ifnar1−/− mice and blood was collected from day 1 to day 9 post-immunization on every other day to measure the ZIKVE gene by qRT-PCR. The results showed that the control mice generated greater levels of viral RNA than the mice who received the plasma infusion at all time points except day 1 post-immunization (FIG. 7C). Thus, the adoptive transfer study indicated that a single dose of plasma collected from Z7-immunized mice is sufficient to significantly inhibit ZIKV replication to achieve 100% survival in Ifnar1−/− mice.

Z7-Based SAM Replicons are More Efficient than VEEV-Based SAM Replicons in Driving GFP Expression In Vitro

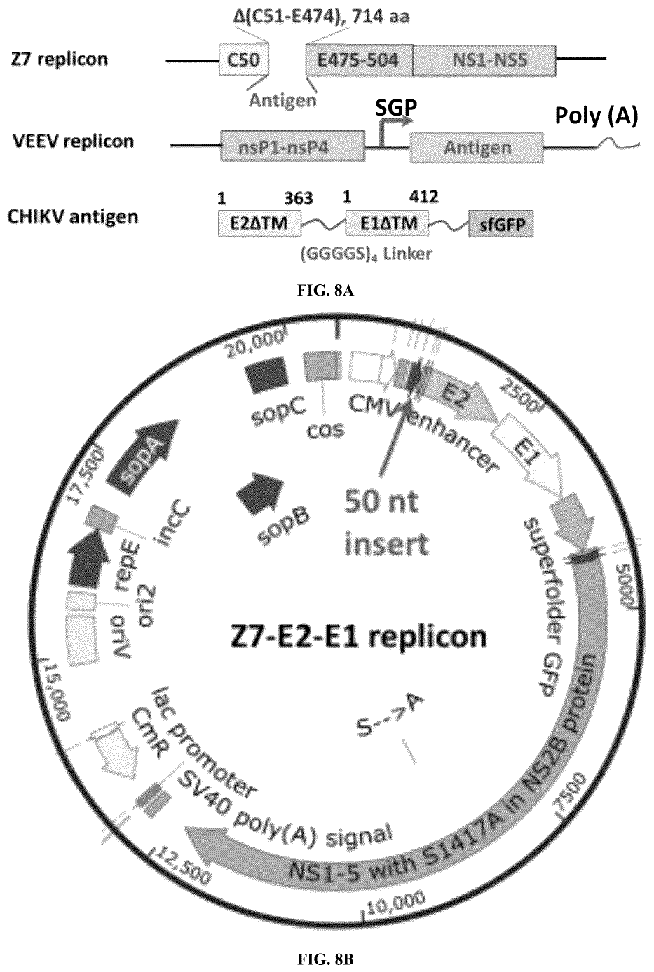

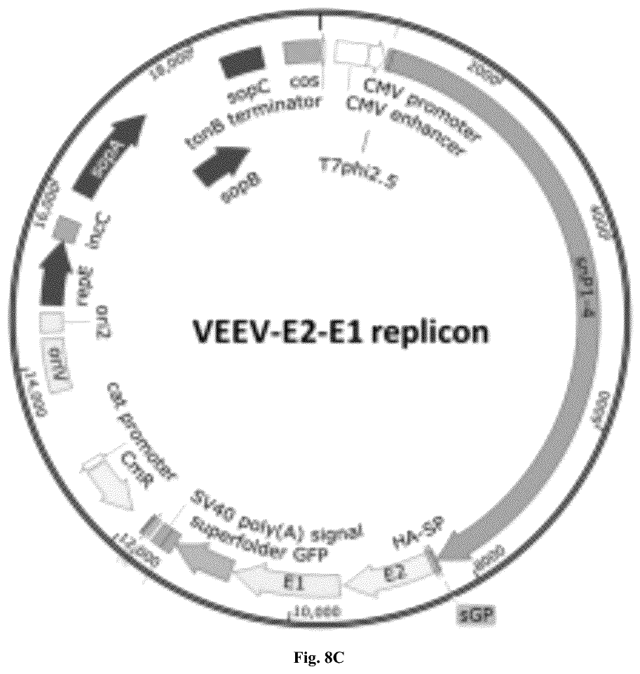

Z7-based SAM replicons were constructed by removing the structural genes C, prM, and E while maintaining the first 150 bp of C (50 aa) and the last 90 bp of E (30 aa) to facilitate optimal RNA replication and polypeptide cleavage to generate a mature NS1-5 (FIGS. 8A and 8B). VEEV-TC83-based SAM replicons were also constructed as illustrated in FIGS. 8A and C. Both Z7 and VEEV-based replicons contain CHIKV E2-E1 proteins and sfGFP as an antigen expression marker. The plasmid DNA sequences of the constructed replicons were verified by next-generation sequencing.

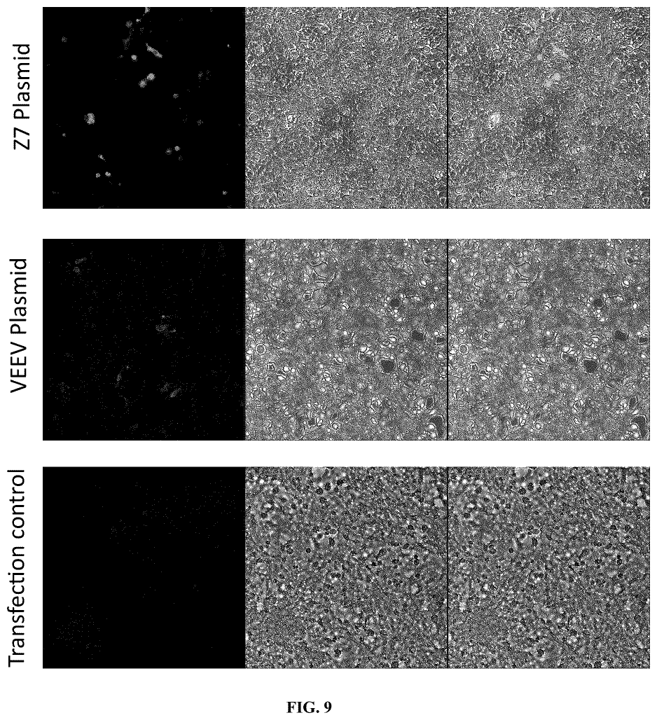

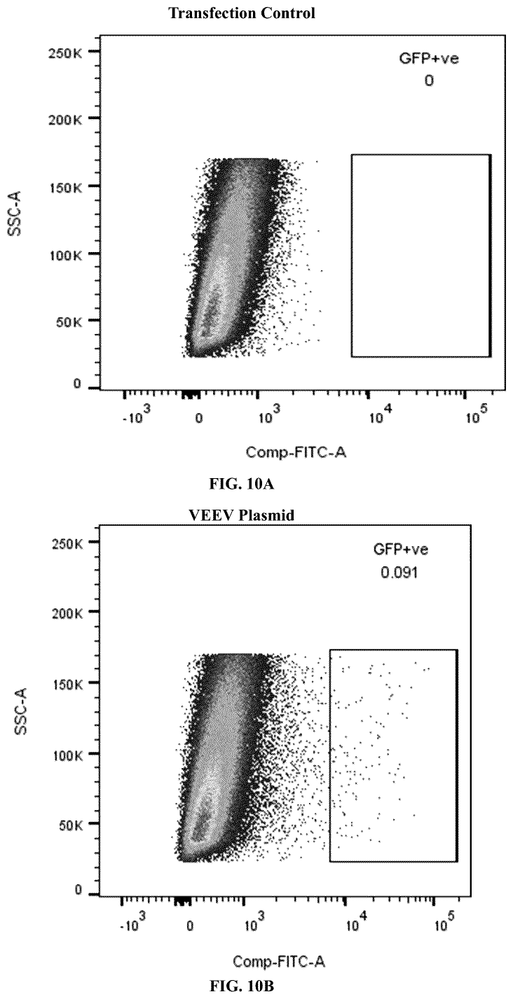

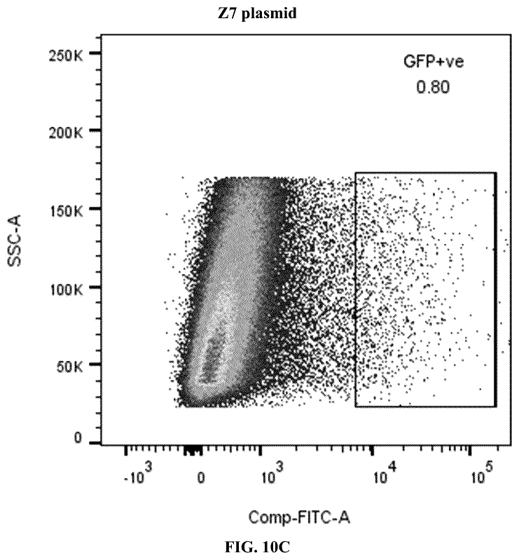

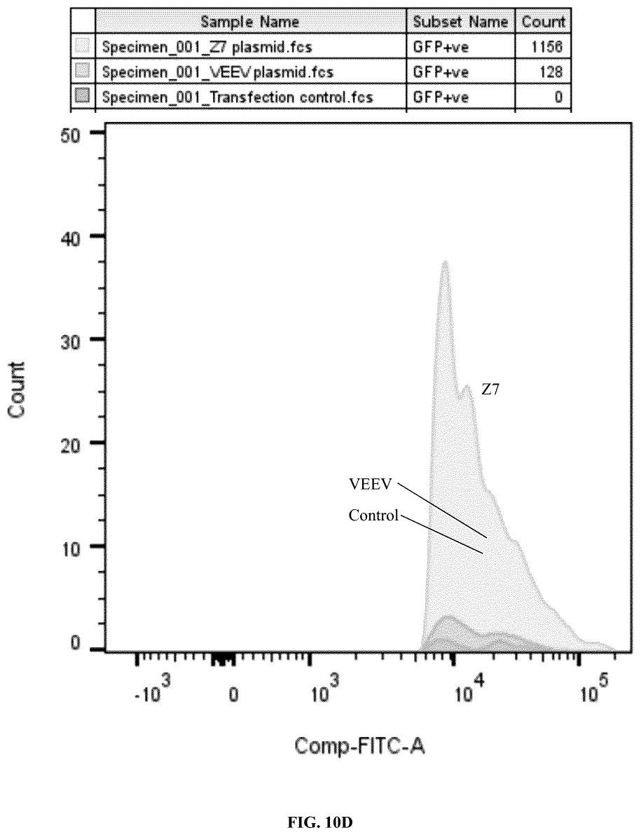

To determine the efficiency of SAM replicons in replication and translation in host cells, an equivalent molar amount of Z7- and VEEV-based SAM were transfected into HEK293 cells (a human cell line that is commonly used for in vitro gene expression analysis) with Lipofectamine TM 3000 reagent (Thermo Fisher Scientific). The transfection reagent alone was used as a control. The GFP expression was analyzed under a fluorescence microscope and flow cytometry at 48 h post-transfection. FIG. 9 shows HEK-293 cells transfected with Z7-based—E2-E1—replicon plasmids expressed more intensive GFP signals than those transfected with VEEV-based—E2-E1—replicons, as viewed under a confocal microscope. FIG. 10 shows approximately 8-fold more GFP-expressing HEK-293 cells transfected with Z7-based—E2-E1-replicon plasmids than those transfected with VEEV-based—E2-E1—replicons, as quantified by flow cytometry.

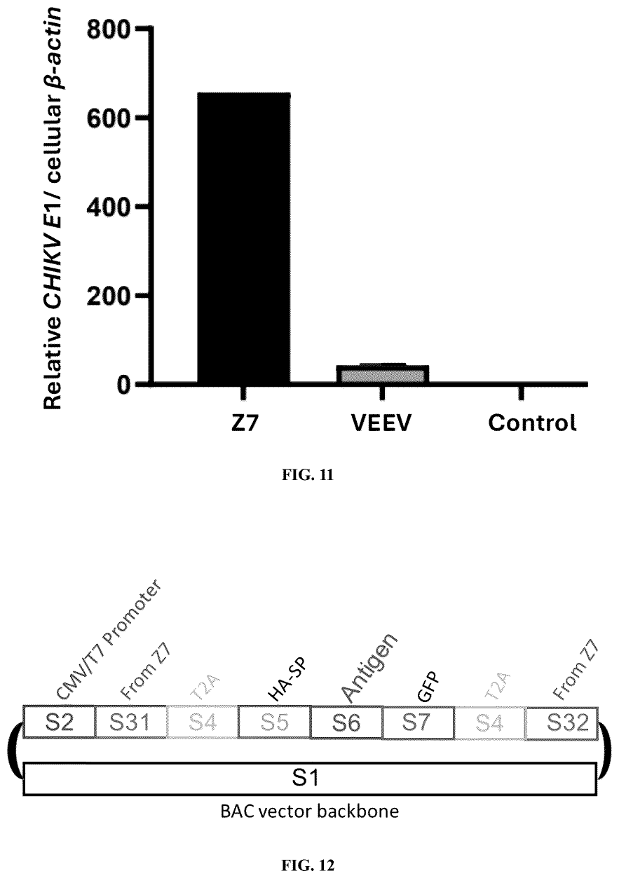

FIG. 11 shows that HEK-293 cells transfected with Z7-based—E2-E1—replicon plasmids expressed approximately 10 times more CHIKV E1 mRNA than those transfected with VEEV-based—E2-E1—replicons, as determined by quantitative real-time PCR (QPCR). These confocal, flow cytometry, and QPCR results demonstrated that Z7-based SAM replicons are more efficient than VEEV-based SAM replicons in driving antigen expression in vitro.

### Discussion

The present invention generates mutant ZIKVs by inserting GC-rich sequences after the SLB region of the 5′ UTR of the ZIKV genome of the pre-pandemic Cambodian strain, FSS13025. The inserts of 38 nucleotides (Z5) and 50 nucleotides (Z7) resulted in the formation of new hairpin structures in the region of 5′ UTR. These new hairpin structures may interfere with the functions of 5′ UTR and inhibit viral genome replication and protein translation because the cellular translation enzymes depend on stem-loop structures to begin replication, and ribosomes might stall when engaged with additional stem loop and hairpin structures^{31,32}.

The enlarged loop in the SLB hairpin structure resulting from the 18-nucleotide insertion (Z3) might significantly interrupt the ZIKV genome processing and translation of the viral proteins, and thus could become lethal to ZIKV. Based on the viability of the mutants, Z7 was selected to further characterize and evaluate its potential as a live-attenuated vaccine candidate. Compared to Z1, the WT control, the replication rate of Z7 was lower; however, through continuous passaging in Vero cells, its titer gradually increased, indicating that Z7 has adapted fitness mutations by continuous replication.

The whole genome sequencing of the mutants confirmed that the insert remained stable at the exact location even after 10 generations. Additionally, a mutation, S1417A was identified in NS2B protein. The NS2B protein couples with NS3 protease to cleave viral proteins and facilitate NS3 proteolytic activity, which contributes to the host cell apoptosis and neuropathogenesis^{21,59}. It has been reported that mutations in NS2B significantly alter the interaction between NS2B and NS3, followed by a decrease in the NS3 protease activity and ZIKV replication⁶⁰. Z7 acquired the S1417A mutation in NS2B, which might also affect NS3 protease.

It has been well-documented that ZIKV NS5 protein binds to and degrades human signal transducer and activator of transcription (STAT) 2 proteins downstream of type I INF receptor signaling, inhibiting the expression of IFN-stimulated genes and induction of innate antiviral responses. Consequently, ZIKV evades host defense mechanisms and can efficiently replicate in human cells. ZIKV NS5 protein cannot target murine STAT2 to evade IFN signaling, which hinders the use of WT mice in studying ZIKV-induced diseases⁶¹⁻⁶³. Therefore, Ifnar1−/− mice have been commonly used to study the pathogenesis of ZIKV and to test antiviral vaccines in vivo. To characterize the infectivity profile of Z7 in vivo, Ifnar1−/− mice were infected with Z7 (G10 or 11) or Z1 as a control. The relative change in body weight and the survival experiments indicated that Z7 did not cause any sign of sickness in Ifnar1−/− mice. Also, lower levels of viral load in blood and the peripheral tissues could be detected in Z7-infected mice.

These results thus confirmed that Z7 has an attenuated infectivity compared to its parental strain, Z1, making it a safe candidate for developing ZIKV-based replicons for SAM. FV replicons may be superior to AV replicons when constructing SAM vaccines to elicit both humoral and cellular immune responses. In the present application, it has been demonstrated that Z7-based SAM replicons are more efficient than VEEV-based SAM replicons in driving antigen expression in vitro.

### EXAMPLES

### Example 1

FIG. 12 shows a schematic structure of the bacterial plasmid used for coning Z7-based SAM vaccine of the present invention. The components of the Z7-based SAM vaccine shown in FIG. 12 are as follows.

Si is a cloning vector backbone derived from a bacterial artificial chromosome (BAC) pCC1BAC. This backbone permits the construction of a variety of plasmids (large or small) and the production of stable DNA sequences with a low probability of mutation due to plasmid-induced toxicity to host E. coli cells. An examplary cloning vector is SEQ ID NO: 23.

S2 is a novel dual CMV/T7 promoter fusion for the initiation of replicon RNA by RNA polymerases either in vivo (host cell enzymes) or in vitro by T7 RNA polymerase. This feature allows the same plasmid to be used directly in the host cell to launch the self-amplifying mRNA (SAM) vaccine (similar to DNA vaccine but with RNA-amplifying capability) or to be used as a template to make RNA in vitro by RNA polymerase (similar to mRNA vaccine but with RNA-amplifying capability). An example of the CMV/T7 promoter is SEQ ID NO: 20. Further examples of the promoter that include the first two nucleotides, AG and GG, of SAM-RNA transcripts are SEQ ID NO: 11 and SEQ ID NO: 12.

S31 and S32 are two pieces of DNA sequences of the newly developed Z7 genome (PMID: 37005424), which was modified from a natural Zika virus genome (a pre-epidemic ZIKV Cambodian strain, FSS13025, Genbank: KU955593.1) and adapted during multiple passages.

S31 contains the 5′ UTR, the 50 nt insert just before the transcription start site (TSS) and the first 50 amino acids (AAs) of the capsid protein (C50). An example of S31 is SEQ ID NO: 21.

S32 includes the last 30 AAs of the envelope protein (E30) and all the non-structural genes NS1-5 (which includes a S1417A in NS2B protein from viral adaptation during the multiple passages), 3′ UTR, and a HDV ribozyme sequence (HDVr). An example of S32 is SEQ ID NO: 22.

Between S31 and S32 a 2142 nucleotide (nt) sequence encoding the majority of the viral capsid protein (C) and envelope protein (E) of Z7 is removed, thereby eliminating the possibility of forming infectious viral particles.

S4 is a DNA sequence encoding “protein-cleaving” peptide sequence (T2A) derived from mouth and foot disease virus. The sequence allows the ribosome to pause, start and re-initiate translation at the site, effectively “cleaving” the long peptide into 2 separate peptide chains. This feature permits the separation of antigen from the rest of Z7-encoded protein sequences. An example of the T2A peptide sequence is SEQ ID NO: 13.

S5 is a DNA sequence encoding signal peptide that allows the secretion of the expressed antigen from a host cell. Haemagglutinin signal peptide (HA-SP) from the influenza virus (SEQ ID NO: 14) is an example, but many other known signal peptide sequences may be used.

S6 is a DNA sequence (SEQ ID NO: 18) encoding the E1 and E2 proteins of Chikungunya virus (strain LR2006_OPY1, GenBank: DQ443544.2) connected by a peptide linker (SGGGSGGGSGGGSGGG (SEQ ID NO: 17)). The 2 proteins form a dimer then form a trimer (spike) which interacts with the host receptor to gain cell entry. An example of the E1 protein of Chikungunya virus is SEQ ID NO: 15. An example of the E2 protein of Chikungunya virus is SEQ ID NO: 16. An example of S6 including the E2 protein (SEQ ID NO: 16) linked via the peptide linker (SEQ ID NO: 17) to E1 protein (SEQ ID NO: 15) is SEQ ID NO: 18.

The S6 component can be replaced by other DNA sequences encoding proteins of other viral strains or cancer antigens.

S7 is a DNA sequence encoding a GFP protein to allow easy detection and analysis of antigen expression. An example of this DNA sequence encoding a GFP protein is SEQ ID NO: 24 Other fluorescent proteins, luciferase propteins, and commonly used antigen epitopes may be used for the purpose of detecting and analyzing SAM RNA expression.

S6 contains one or more application specific antigens (antiviral or anticancer). Some of S4-S7 may be omitted, and S4-S7 can be arranged in different ways depending on specific applications.

### Example 2

### Materials and Methods

### Cells and Viruses

The in vitro experiments were performed on Vero (ATCC CCL-81) or HEK-293 cells (ATCC CRL-1573) maintained in Dulbecco's Modified Eagle's Medium (DMEM, Life Technologies) supplemented by 1% Penicillin/Streptomycin (P/S, Gibco), and 10% Fetal Bovine Serum (FBS, Atlanta Biologicals). The cells were kept in an incubator at 37° C. with 5% CO₂ and a relative humidity of about 95%. The ZIKV Puerta Rico strain (PRVABC59, GenBank number KU501215) was obtained from B. Johnson (CDC Arbovirus Branch), was propagated in Vero cells, and was quantified by plaque assay. The ZIKV plasmids of the Cambodian strain (GenBank number KU955593.1) were generously provided by Dr. Pei-Yong Shi at the University of Texas Medical Branch (UTMB) and were used for generating Z1.

### Plasmid Construction, Transmission, and Virus Collection

A pFLZIKV plasmid containing the Cambodian wild type ZIKV sequence FSS13025 (GenBank number KU955593.1) with the pACYC177 backbone and a pCC1BAC-PRV plasmid^{65,66} was cloned. The cloning strategy involved adding a cytomegalovirus (CMV) promoter to the 5′ UTR of WT Z1 sequence in order to generate the FSS13025 viral RNA and changing the backbone from pACYC177 to pCC1BAC to facilitate plasmid propagation in E. coli. Three overlapping DNA fragments (F1-pCC1BAC backbone, F2-CMV-5′UTR-C-prM-E, and F3-E-NS1-NS5-3′UTR-HDVr-SV40 poly(A) signal) were generated by PCR using the DNA polymerase Q5 (NEB), and were then assembled into the Z1 plasmid by NEBuilder HiFi DNA Assembly (NEB) into a full size 19,482 bp plasmid containing the Z1. The major portion of the viral sequence (E-NS1-NS5-3′UTR-HDVr) was first cloned into a plasmid Fb to add SV40 poly(A) signal by in vivo cloning³⁵. Fb was then used as the template to generate F3. Detailed information is shown in Table 2.

TABLE 2

| DNA | | Primer | |
|---|---|---|---|
| Name | Template | Name | Primer Sequence |
| F1 | pCC1BAC- | P1f | AGCTTGGCGTAATCATGGTCATAGCTG |
| | PRV | | (SEQ ID NO: 1) |
| | | P1r | GATCGGCACGTAAGAGGGGACTTCCAT |
| | | | TGTTCATTCCACG (SEQ ID NO: 2) |
| F2 | F21/F22 | P2f | AATGAACAATGGAAGTCCCCTCTTACG |
| | | | TGCCGATCAAGTC (SEQ ID NO: 3) |
| | | P2r | GGGTTAGCGGTTATCAACCTCCCAACT |
| | | | (SEQ ID NO: 4) |
| F21 | pcDNA3 | P2f | AATGAACAATGGAAGTCCCCTCTTACG |
| | Kan | | TGCCGATCAAGTC (SEQ ID NO: 5) |
| | | P21r | CGGTTCACTAAACGAGCTCTGCTTATA |
| | | | TAGACCTCCCA (SEQ ID NO: 6) |
| F22 | pFLZIKV | P22f | GCAGAGCTCGTTTAGTGAACCGAGTTG |
| | | | TTGATCTGTGTGA (SEQ ID NO: 7) |
| | | P2r | GGGTTAGCGGTTATCAACCTCCCAACT |
| | | | (SEQ ID NO: 4) |
| F3 | Fb | P3f | GGACCTTGCAAGGTTCCAGCTCAGA (SEQ |
| | | | ID NO: 8) |
| | | P3r | GTGTGAAATACCCCGAACCCATGATCCT |
| | | | (SEQ ID NO: 9) |

Vero cells were plated at 2.5×10⁵ cells/well in 6-well plates and incubated for 24 h at 37° C. with 5% CO₂. Z1, Z3, Z5, and Z7 plasmids (500 ng) were transfected with Lipofectamine 3000 reagent (ThermoFisher Scientific) according to the user manual. The Vero cells were allowed to incubate at 37° C. with 5% CO₂ for 5 days to collect the cell culture supernatant containing the WT (Z1) and mutant (Z3, Z5, and Z7) viruses. To continuously pass the Z7 on Vero cells, 150 μl of the virus-containing medium from the previous passage was inoculated into 2.5×10⁵ Vero cells in 6-well plates for 5 days. The culture supernatant from each generation was collected and stored in a −80° C. freezer. The viral titers of Z1, Z5, and Z7 (G10 and 11) were determined by FFA.

### Mice and Animal Study

Breeding pairs of Ifnar1−/− mice in C57BL/6J background (Stock #028288) were purchased from the Jackson Laboratory. The breeding pairs and pups were kept in a clean room, and the infection experiments were performed in an animal BSL-3 lab at the University of Southern Mississippi. For ZIKV infection, four-week-old Ifnar1−/− mice were subcutaneously injected on the ventral side of the left hind footpad with 1×10⁵ FFU of Z1 or Z7 (G10) in phosphate buffered saline (PBS) on day 0. The body weight of the infected mice was measured daily for 8 days post-immunization. Blood samples were collected on alternate days from day 1 to day 9 post-immunization in 0.5M ethylenediaminetetraacetic acid (EDTA) from the retro-orbital sinus under isoflurane anesthesia, and the levels of ZIKVE gene were measured by qRT-PCR. To measure the level of anti-ZIKV E IgG by ELISA (Alpha Diagnostic International), blood samples were collected on day 0 and day 24 post-immunization to prepare plasma. On day 42 post-immunization, the mice were challenged with 1×10⁵ PFU of ZIKV (strain PRVABC59), and blood was collected from day 1 to day 3 p.c. to measure the viremia by qRT-PCR and plaque assay. On day 3 p.c., the mice were euthanized, and the livers and spleens were collected to measure the viral load by qRT-PCR. Data were normalized by mouse β-actin as a housekeeping gene.

Quantitative Reverse Transcription Polymerase Chain Reaction (qRT-PCR)

The total RNA was extracted from cell culture and tissue samples using TRI Reagent (Molecular Research Center, Inc). The RNA was converted into the first-strand complementary DNA (cDNA) using an iSCRIPT™ cDNA synthesis kit (Bio-Rad). The qRT-PCR assays were performed in a CFX Connect Real-Time System (Bio-Rad) using iTaq™ Universal Probes Supermix (Bio-Rad) for the detection of ZIKVE^{67,68}, and cellular β-actin⁶⁹. ZIKV genome copies were calculated by measuring ZIKVE with qRT-PCR following the previous protocol³⁹.

### Viral Growth Kinetics

Vero or HEK-293 cells were plated at 5×10⁵ or 2.5×10⁵ cells/well, respectively, in 12-well plates and incubated overnight at 37° C. with 5% CO₂. The cells were then infected with 0.1 MOI (Vero cell) or 0.01 MOI (HEK-293 cell) of Z1 or Z7 (G11) and incubated for 1 h. The medium was replaced with 1 ml of DMEM supplemented with 10% FBS, and 1% P/S, and incubated for 6 days. The culture supernatants containing either Z1 or Z7 were collected daily from day 1 to day 6. The viral titers of Z1 and Z7 were determined by FFA.

### Cytopathic Effect Assay (CPE)

Vero cells were plated at 5×10⁴ cells/well in 12-well plates and incubated overnight at 37° C. with 5% CO₂, then infected with 0.1 MOI of Z1 and Z7 (G11) and incubated for 1 h at 37° C. The medium was then replaced with 1 ml of DMEM supplemented with 10% fetal bovine serum (FBS) and 1% P/S and incubated for 3 days. On day 3 post-immunization, the cells were stained using LIVE/DEAD Cell Imaging Kit (488/570, ThermoFisher Scientific) according to the user manual. The images were taken using a Leica M165 FC microscope (Leica Microsystems).

### Immune Fluorescence Assay (IFA)

Vero cells were plated at 1.25×10⁴ cells/well in 24-well glass-bottom plates and incubated overnight at 37° C. with 5% CO₂. The cells were then infected with 0.1 MOI of Z1, Z7 (G11), or PBS as a control and incubated for 1 h at 37° C. After incubation, the cell medium was replaced with 500 μl of DMEM, and the cells were incubated at 37° C. for 3 days. The cells were then fixed with 250 μl of 4% para-formaldehyde solution (PFA) in PBS for 15 min at room temperature (RT), followed by 2× wash with PBS. The cells were permeabilized with 250 μl of 0.1% Triton X for 20 min at room temperature (RT) followed by 2× wash with PBS, then blocked with 500 μl of 5% skim milk in 0.1% PBST (PBS with Tween®-20) for 1 h at 4° C. The cells were then probed with the mouse anti-flavivirus glycoprotein E IgG antibody (4G2, in-house produced from the hybridoma D1-4G2-4-15 HB-112, ATCC), diluted with 5% skim milk in 1:50 ratio, at 200 μl/well, incubated overnight at 4° C. followed by two 5-minute washes with 0.1% PBST. The cells were then probed with goat anti-mouse IgG conjugated with Alexa Fluor 488 (Abcam), diluted with 5% skim milk in 1:100 ratio, at 200 μl/well, at 4° C. in the dark for 2 h, followed by two 5-minute washes with 0.1% PBST. The nuclei of the cells were stained with 600 nM DAPI solution at RT for 10 min. The images were taken using a Stellaris STED confocal microscope (Leica Microsystems).

### Focus Forming Assay (FFA)

The FFA assay was developed by modifying a previously described protocol⁷⁰. Vero cells were plated at 5×10⁵ cells/well in 12-well plates and incubated overnight at 37° C. with 5% CO₂. The cells were inoculated with serially diluted viruses Z1 or Z7 (G10 or G11) and incubated for 2 h at 37° C. Then the medium was replaced with 1 ml/well of 1×Opti MEM GlutaMAX (Gibco) medium supplemented with 1% methylcellulose (Sigma), 10% FBS, and 1% P/S, and incubated for 3 days (Z1) or 4 days (Z7). After the incubation, the overlay medium was removed and washed gently with PBS. The plates were fixed with 4% PFA for 15 min, permeabilized with 0.1% Triton X for 20 min at RT and blocked with 5% skim milk for 1 h. The cells were then probed with 4G2 antibody diluted with 5% skim milk in 1:50 ratio, incubated at 4° C. overnight in the dark followed by two 5-minute washes with 0.1% PBST. The cells were then probed with goat anti-mouse IgG conjugated with horseradish peroxidase (HRP) (Abcam), diluted with 5% skim milk in 1:500 ratio, incubated at 4° C. in the dark for 2 h, followed by two 5-minute washes with 0.1% PBST, and air dried for 20 min at RT. The immune-positive foci were developed with TrueBlue peroxidase substrate (KPL, Sera care).

### Plaque Assay

Vero cells were plated at 6×10⁵ cells/well in 6-well plates and incubated overnight at 37° C. with 5% CO₂. One ml of serially diluted ZIKV (strain PRVABC59), Z1, or Z7 were incubated with the Vero cells monolayer for 1 h at 37° C. Then the virus-containing medium was replaced with DMEM overlay medium containing 1% SeaPlaque Agarose (SPA, Lonza), 10% FBS, and 1% P/S and incubated for 5 days. The plaques were stained with 0.1% Neutral red solution (Sigma) and counted⁶⁸. The plaque size on day 5 post-immunization was measured by ImageJ software (National Institute of Health) with the ViralPlaque add-in 71 and expressed as area in pixels.

### Plaque Reduction Neutralization Test (PRNT)

Vero cells were plated at 6×10⁵ cells/well in 6-well plates and incubated overnight. The plasma samples were incubated at 56° C. for 30 min to inactivate the complements, serially diluted from 101 to 107 fold with pre-warm, serum-free DMEM. The diluted samples were mixed with 100 PFUs of ZIKV (strain PRVABC59) and incubated at 37° C. for 1 h. The virus-plasma mixtures were applied onto Vero cells and incubated for 1 h. Then the virus-containing medium was replaced with DMEM overlay medium containing 1% SeaPlaque Agarose (SPA, Lonza), 10% FBS, and 1% P/S and incubated for 4 days. The plaques were stained with 0.1% Neutral red solution (Sigma) and counted⁶⁸. Then the PRNT50 values were calculated with GraphPad Prism software (version 7.0)⁷².

### Flow Cytometry

Seven-week-old Ifnar1^{-/-} mice were infected with 1×10⁵ FFU of Z7 (G10) or PBS (control). Mice were euthanized on day 8 post-immunization to collect splenocytes. The splenocytes (3×10⁶) were plated in 6-well plates and restimulated with 0.1 MOI of Z1 for 24 h at 37° C. During the final 8 h of the stimulation, Brefeldin A solution (BD Bioscience) was added at 1:1000 to block cytokine secretion. Cells were then stained with antibodies against mouse CD3 (FITC-conjugated, BD Bioscience), CD4 (PerCp Cy 5.5-conjugated, BD Bioscience, and CD8 (APC-conjugated, BD Bioscience). Cells were then fixed with 2% PFA overnight and permeabilized with 1× Permeabilization buffer. Cells were then intracellularly stained with PE-Cy7 conjugated anti-IFNγ (BD Bioscience).

HEK-293 cells (80-90% confluent cells) grown in 6-well plates were transfected with 2.5 μg of plasmids with 7.5 μL/well of Lipofectamine TM 3000 reagent. The cells were incubated at 37° C. with 5% CO₂ for 2 days, then for GFP (FITC positive cells) detection by flow cytometry. Data were acquired using BD LSR Fortessa and analyzed with FlowJo (v10.8.0.) software.

### Transmission Electron Microscopy

Z1 and Z7 (G9) virus samples were centrifuged at 5,000 rpm for 5 min at 4° C. to remove cell debris. Viral samples were initially added to the top of a 20% sucrose cushion in Polyallomer ultracentrifuge tubes (Beckman Coulter Life Sciences) and were centrifuged by an Optima XPN-80 Ultracentrifuge (REVCO) at 28,000 rpm for 2 h at 4° C. The pellets were then resuspended and fixed with 4% glutaraldehyde solution (Sigma). The virus samples were negatively stained with 2% Uranyl acetate solution and visualized under a Transmission Emission Microscope (TEM)³⁸. The TEM images of Z1 and Z7 were taken by JEOL JEM-1400 120 kV TEM machine in the Shared Instrumental Facility of Louisiana State University (Baton Rouge, LA).

### Next Generation Sequencing

Vero cells were plated at 2×10⁵ cells/well in 6-well plates, incubated overnight at 37° C. with 5% CO₂ and infected with 0.5 MOI of Z7 (G8-10) or Z1 for 48 h. Total RNA was extracted using an RNeasy Mini Kit (Qiagen) for RNA sequencing using the Illumina platform (Psomagen). The paired reads were processed to generate de novo sequences which were aligned to the ZIKV sequence (Cambodian strain, FSS13025, GenBank number KU955593.1).

### Statistical Analysis

Data were compared using Mann Whitney U test, log-rank test, or unpaired Student's t-test with GraphPad Prism software (version 7.0), whichever was applicable.

These examples show a comparison of AV- and FV-SAM replicons in vitro and in vivo and characterize their immunogenic potency and safety profiles based on previously established cell culture and mouse models.

### Example 3

### Development of Z7-Based SAM Replicons for CHIKV Antigens

Both male and female mice have demonstrated similar susceptibility to CHIKV infection, therefore, approximately equal numbers of male and female mice were tested in Example 2.

Aim 1. Construct Z7-Based RNA Replicons Targeting CHIKV E2-EJ and Evaluate their Efficiency at the Transcriptional and Translational Levels in Vitro.

The single-stranded CHIKV RNA genome consists of two open-reading frames (ORF) that encode four nonstructural proteins (nsP1-4) required for virus replication and five structural proteins (C, E3, E2, 6K, and E1), respectively [275]. Of these structural proteins, heterodimer E2-E1 proteins, the main component of the viral surface glycoproteins, mediate the cellular receptor attachment and the membrane fusion during the viral entry [220]. Pre-clinical vaccines targeting CHIKV E2-E1 have been documented to elicit high levels of neutralizing antibodies, as well as specific Interferon-gamma (IFN-γ)-producing cells and polyfunctional CD4⁺ and CD8⁺ T cells[276, 277]. Herein, a Z7-based SAM vaccine targeting CHIKV E2-E1 glycoproteins was developed. Flavivirus replicons might be superior to Alphavirus (AV) replicons when constructing SAM vaccines to elicit both humoral and cellular immune responses. However, these two SAM replicons have never been directly evaluated side-by-side. Thus, we demonstrated herein a comparison of CHIKV E2-E1 SAM replicons with VEEV-TC83 nonstructural genes and Z7-based SAM.

Aim 1a. Construct Z7- and VEEV-TC83-Based SAM Replicons Expressing CHIKV E2-E1-sfGFP.

Z7-based SAM replicons were constructed by removing its structural genes Capsid (C), Pre-membrane (prM), and Envelope (E) while maintaining the first 150 base pairs (bp) of Capsid (50 amino acids (aa)) and the last 90 bp of the Envelope (30 aa) to facilitate optimal RNA replication and polypeptide cleavage to generate mature NS1-5 [48, 63](FIGS. 8A and 8B). VEEV-TC83-based SAM replicons will be constructed as illustrated in FIGS. 8A and 8B. The antigenic DNA sequences of the constructed replicons were verified by next-generation sequencing. The resulting DNA was used as the template to prepare 5′ capped RNA replicons by in vitro transcription in the presence of T7 RNA polymerase and m⁷GpppA as done in Example 1, according to our established protocol [59]. After the in vitro assessment of the superfolder Green Fluorescent Protein (sfGFP)-containing SAM replicons (Aim 1b), we generated a set of new SAM replicons targeting CHIKV E2-E1 proteins without sfGFP, which are discussed in Aim 2.

Aim 1b. In Vitro Assessment of the Replication and Translation of the SAM Replicons.

To determine the efficiency of SAM replicons in replication and translation in host cells, an equivalent molar amount of Z7- and VEEV-based SAM were transfected into HEK293 and NIH3T3 cells (human and mouse cell lines that are commonly used for in vitro gene expression analysis) with EndoFectin Max (Genecpopeia). The empty replicons (without CHIKV antigens) and the transfection reagent alone were used as controls. The sfGFP expression was analyzed under a fluorescence microscope at 24 h or 48 h after transfection. The replication and translation of the SAM replicons were measured by qRT-PCR and immunocytochemistry, respectively. To analyze replicon transcription efficiency, qRT-PCR was designed to determine the levels of negative-strand and positive-strand RNA E2 and NS5 or nsP4, the RNA-dependent RNA polymerase of Z7 and VEEV, respectively [64]. The monoclonal antibodies (mAbs) against CHIKV E2 and E1 proteins (Thermo Fisher) were used to analyze cellular expressed viral antigens by different approaches of immunocytochemistry, for example, Western blot, immunostaining, or flow cytometry. In addition, type I IFNs (α and β) in the cell culture media will be measured by ELISA. All methods for these assays are routinely used in our labs [274, 280-281].

The in vivo cloning method is simple, fast, and accurate. The sfGFP reporter SAM replicons included E2-EJ since the sfGFP itself may be too short to reflect the efficiency of the replication and translation of large antigens, i.e., E2-E1. While synthetic mRNA or plasmids can be effectively transfected with chemical reagents (such as with EndoFectin, the transfection efficiency of SAM may be lower due to their large sizes. In this case, viral mRNA replicons were delivered to the host cells by electroporation [263]. The transfection of synthetic mRNA is highly effective, but it may result in the potential cytotoxicity caused by the foreign mRNA-induced innate immune responses [283]. Because Z7 NS5 protein significantly inhibits the production of type I IFNs in human cells, Z7-based SAMs to induced lower levels of cytotoxicity than VEEV-based SAMs, albeit non-structural (NS) proteins of FV may also have some inhibitory roles on type I IFNs[284]. In addition to using m⁷GpppA as the 5′ cap to increase translation efficiency [274], the concentration of SAM-replicons that give maximal translation efficiency with minimal cytotoxicity was determined. Transfected cells were monitored for cell morphology and viability at both early (2-4 h) and later time points (12h, 24h, and 48 h) with our established methods [274, 275].

### Aim 2. Evaluate the Immunogenicity and Safety of Z7- and VEEV-Based SAM Replicons in Mice

Immunocompetent adult mouse models were determined to be valuable systems both for studying the pathogenesis of CHIKV-induced arthritis and for testing CHIKV vaccines and therapies. Subcutaneous CHIKV infection in the footpad of C57BL/6 mice resulted in a biphasic swelling response in the inoculated foot, with peaks occurring approximately 3 and 6 days after infection [285, 286]. The inoculated foot developed severe arthritis, tendonitis, and fasciitis[285-287]. Type I IFN signaling has been shown to play an essential role in controlling infections of alphaviruses, including CHIKV[288]. Mice lacking a functional type I IFN receptor or other key components of the type I IFN pathway (e.g., IRF3/IRF7) are highly susceptible to CHIKV infection [287, 288]. Therefore, both wild-type C57BL6 and Ifnar1^{-/-} mice were used to test the efficacy of CHIKV SAM vaccines. One concern regarding ZIKV vaccine, is that it may prime cross-reactive antibodies to enhance natural infections of the antigenically related flaviviruses, such as dengue virus (DENV), via antibody-dependent enhancement (ADE). It has been documented that cross-reactive antibodies targeting the conserved fusion loop epitope in the E protein domain II are the primary source of the ADE[289]. Although the fusion loop region gene has been removed from the Z7 SAM replicon (FIG. 8A), and no infectious ZIKV will be produced by SAM replicons, immune responses to ZIKV NS proteins are also expected in mice and humans. In addition, an adenovirus-based CHIKV vaccine has been shown to enhance infection of mayaro virus (MAYV), a closely related alphavirus co-circulating with CHIKV in the Americas, indicating a potential ADE concern for the CHIKV vaccine development[290].

Aim 2a. Measure Humoral and Cellular Responses of CHIKV SAM Replicons in Mice

7-week-old C57BL/6 and Ifnar1^{-/-} mice (6 males and 6 females/group) were immunized with 1 μg or 5 μg of Z7- or molar equivalent VEEV-based CHIKV SAM lipid nanoparticle complexes (Packgene) via intramuscular injection (i.m.) injection in the thigh muscles of the hind limb. The control groups received the same amount of empty replicons (absence of CHIKV E2-E1). The mice were monitored and weighed daily. Blood samples were collected on days 0, 7, 14, 21, 28, and 50 post-immunization for anti-CHIKV IgG productions in the plasma by Enzyme-Linked Immunosorbent Assay (ELISA). In addition, a plaque reduction neutralization test (PRNT) was performed to assess if the antisera neutralize CHIKV in Vero cells and to determine the EC₅₀. In addition to the humoral responses, T cell responses against CHIKV antigens were evaluated. Half the number of the mice (3 males and 3 females/group) were euthanized on day 14, post-immunization, to collect the spleens to make single-cell suspensions. The splenocytes were stimulated for 20 h with pools of 15-mer peptides (Genscript) spanning the CHIKV E2-E1 proteins overlapping by 11 amino acids for IFN-γ, IL-4 and IL-17A ELISpot and flow cytometric analysis of CD3⁺CD4⁺IL4⁺, CD3⁺CD4⁺IL12⁺, CD3⁺CD4⁺IFN-γ⁺, CD3⁺CD8⁺TNF-α⁺, and CD3⁺CD8⁺IFN-γ⁺ populations. The rest of the animals were euthanized on day 50, post-immunization, to evaluate the memory CD8⁺ T cell responses.

Aim 2b. Evaluate Whether CHIKV SAM-Induced Immunity is Sufficient to Protect Mice from CHIKV Infection.

7-week-old Ifnar1^{-/-} mice (6 males and 6 females/group) were immunized with 1 μg or 5 μg of Z7- or molar equivalent VEEV-based CHIKV SAM replicons lipid nanoparticle complexes (Packgene) according to the results of Aim 2a or the empty replicons via i.m. injection. Similarly, the blood samples were collected on day 0, 7, 14, and 21 post-immunization for anti-CHIKV IgG productions in the plasma by ELISA. On D21 post-immunization, the mice were infected with CHIKV (strain LR2006 OPY1, 10⁵ PFUs in 50 μl PBS) via footpad (subcutaneous) inoculation. The body weight and thickness of the inoculated footpad were measured daily for 10 days [285, 286]. The blood samples were collected on days 1, 2, and 3 post-infection (p.i.) for the viral load measurement by qRT-PCR and plaque assay, and cytokines and chemokines (IFN-α, IFN-0, IFN-γ, TNF-α, CXCL1, CXCL2, CXCL10, IL-10, IL-2, IL-4, IL-10, IL-12, and IL-17a) in the plasma, as measured by a Luminex assay. The infected mice were euthanized on D1 (3 male and 3 female mice) and D3 (3 male and 3 female mice) post-infection to collect infected footpad, spleen, and brain samples to measure the viral burden by qRT-PCR and plaque assay and perform pathological analysis of the footpad tissues. To evaluate if the plasma collected CHIKV SAM replicon-immunized mice protects against CHIKV infection, 100 μl of plasma collected from either the Z7- or VEEV-based SAM-immunized mice or control mice were transferred via retro-orbital injection in 5-week-old Ifnar1^{-/-} mice (4 males and 4 females/group). The next day, both groups were challenged with 1×10⁵ PFU of CHIKV via footpad. The body weight and thickness of the inoculated footpad was measured as above. The blood samples were collected on days 1, 2, and 3 p.i. for the viral load measurement by qRT-PCR and plaque assay. The mice were monitored daily for survival for 21 days.

Aim 2c. Determine if the SAM Replicons Induce ADE Responses In Vitro.

IgG purification kits (GE Healthcare, PA) were used to purify total IgG from the pooled sera of the Z7- and VEEV-SAM replicon immunized mice (Aim 2a and 2b). DENV (serotypes 1 to 4) and MAYV were incubated with each of eight 3-fold serial dilutions of IgG for 1 h at 37° C. and then added to FcγR expressing cells THP-1 and K562 at an MOI of 1 [291, 292]. The cells and cell media were collected at 48h for analysis of the viral replication by qRT-PCR and plaque assay. [293, 294]

### REFERENCES

All references cited herein are hereby incorporated by reference in their entirety as if fully set forth herein.
1. White, M. K., Wollebo, H. S., David Beckham, J., Tyler, K. L. & Khalili, K. Zika virus: An emergent neuropathological agent.
2. Musso, D. & Gubler Duane, J. Zika Virus. Clinical Microbiology Reviews 29, 487-524 (2016).
3. Morrison, T. E. & Diamond, M. S. Animal Models of Zika Virus Infection, Pathogenesis, and Immunity. J Virol 91(2017).
4. Gorman, M. J. et al. An Immunocompetent Mouse Model of Zika Virus Infection. Cell Host Microbe 23, 672-685 e676 (2018).
5. Lazear, H. M. et al. A Mouse Model of Zika Virus Pathogenesis. Cell Host Microbe 19, 720-730 (2016).
6. Mohr, E. L. Modeling Zika Virus-Associated Birth Defects in Nonhuman Primates. J Pediatric Infect Dis Soc 7, S60-S66 (2018).
7. Mlakar, J. et al. Zika Virus Associated with Microcephaly. N Engl J Med 374, 951-958 (2016).
8. Paul, A. M. et al. Congenital Zika Virus Infection in Immunocompetent Mice Causes Postnatal Growth Impediment and Neurobehavioral Deficits. Front Microbiol 9, 2028 (2018).
9. Prata-Barbosa, A., Martins, M. M., Guastavino, A. B. & Cunha, A. Effects of Zika infection on growth. J Pediatr (Rio J) 95 Suppl 1, 30-41 (2019).
10. Krauer, F. et al. Zika Virus Infection as a Cause of Congenital Brain Abnormalities and Guillain-Barre Syndrome: Systematic Review. PLoS Med 14, e1002203 (2017).
11. Katz, I., Gilburd, B. & Shovman, O. Zika autoimmunity and Guillain-Barre syndrome. Curr Opin Rheumatol 31, 484-487 (2019).
12. Duffy, M. R. et al. Zika virus outbreak on Yap Island, Federated States of Micronesia. N Engl J Med 360, 2536-2543 (2009).
13. Hayes, E. B. Zika virus outside Africa. Emerg Infect Dis 15, 1347-1350 (2009).
14. Counotte, M. J. et al. Sexual transmission of Zika virus and other flaviviruses: A living systematic review. PLoS Med 15, e1002611 (2018).
15. Hastings, A. K. & Fikrig, E. Zika Virus and Sexual Transmission: A New Route of Transmission for Mosquito-borne Flaviviruses. Yale J Biol Med 90, 325-330 (2017).
16. Wilder-Smith, A. & Osman, S. Public health emergencies of international concern: a historic overview. J Travel Med 27 (2020).
17. Morabito, K. M. & Graham, B. S. Zika Virus Vaccine Development. J Infect Dis 216, S957-S963 (2017).
18. Yadav, P. D. et al. Zika a Vector Borne Disease Detected in Newer States of India Amidst the COVID-19 Pandemic.
19. Shan, C., Xie, X. & Shi, P. Y. Zika Virus Vaccine: Progress and Challenges. Cell Host Microbe 24, 12-17 (2018).
20. Shan, C. et al. Zika Virus: Diagnosis, Therapeutics, and Vaccine. ACS Infect Dis 2, 170-172 (2016).
21. Bai, F. & Thompson, E. A. West Nile Virus (Flaviviridae), in Encyclopedia of Virology (Fourth Edition). (eds. D. H. Bamford & M. Zuckerman) 884-890 (Academic Press, Oxford; 2021).
22. Song, Y., Mugavero, J., Stauft, C. B. & Wimmer, E. Dengue and Zika Virus 5′ Untranslated Regions Harbor Internal Ribosomal Entry Site Functions. mBio 10 (2019).
23. Anthony, K. G., Bai, F., Krishnan, M. N., Fikrig, E. & Koski, R. A. Effective siRNA targeting of the 3′ untranslated region of the West Nile virus genome. Antiviral Res 82, 166-168 (2009).
24. Bai, F., Thompson, E. A., Vig, P. J. S. & Leis, A. A. Current Understanding of West Nile Virus Clinical Manifestations, Immune Responses, Neuroinvasion, and Immunotherapeutic Implications. Pathogens 8 (2019).
25. Ye, Q. et al. Genomic characterization and phylogenetic analysis of Zika virus circulating in the Americas. Infect Genet Evol 43, 43-49 (2016).
26. Ng, W. C., Soto-Acosta, R., Bradrick, S. S., Garcia-Blanco, M. A. & Ooi, E. E. The 5′ and 3′ Untranslated Regions of the Flaviviral Genome. Viruses 9 (2017).
27. Lodeiro, M. F., Filomatori, C. V. & Gamarnik, A. V. Structural and functional studies of the promoter element for dengue virus RNA replication. J Virol 83, 993-1008 (2009).
28. Wengler, G., Wengler, G. & Gross, H. J. Studies on virus-specific nucleic acids synthesized in vertebrate and mosquito cells infected with flaviviruses. Virology 89, 423-437 (1978).
29. Filomatori, C. V. et al. A 5′RNA element promotes dengue virus RNA synthesis on a circular genome.
30. Yu, L., Nomaguchi, M., Padmanabhan, R. & Markoff, L. Specific requirements for elements of the 5′ and 3′ terminal regions in flavivirus RNA synthesis and viral replication. Virology 374, 170-185 (2008).
31. Hinnebusch, A. G. & Lorsch, J. R. The mechanism of eukaryotic translation initiation: new insights and challenges. Cold Spring Harb Perspect Biol 4 (2012).
32. Babendure, J. R., Babendure, J. L., Ding, J. H. & Tsien, R. Y. Control of mammalian translation by mRNA structure near caps. RNA 12, 851-861 (2006).
33. Kozyrev, S. V. et al. Structural insertion/deletion variation in IRF5 is associated with a risk haplotype and defines the precise IRF5 isoforms expressed in systemic lupus erythematosus.
34. Endo, K., Stapleton, J. A., Hayashi, K., Saito, H. & Inoue, T. Quantitative and simultaneous translational control of distinct mammalian mRNAs. Nucleic Acids Res 41, e135 (2013).
35. Huang, F. A.-O., Spangler, J. R. & Huang, A. Y. In vivo cloning of up to 16 kb plasmids in E. coli is as simple as PCR.
36. Bradley, M. P. & Nagamine, C. M. Animal Models of Zika Virus. Comp Med 67, 242-252 (2017).
37. Kiesslich, S. & Kamen, A. A. Vero cell upstream bioprocess development for the production of viral vectors and vaccines. Biotechnology Advances 44, 107608 (2020).
38. Boigard, H. et al. Zika virus-like particle (VLP) based vaccine. PLoS Negl Trop Dis 11, e0005608 (2017).
39. Acharya, D., Paul, A. M., Anderson, J. F., Huang, F. & Bai, F. Loss of Glycosaminoglycan Receptor Binding after Mosquito Cell Passage Reduces Chikungunya Virus Infectivity. PLoS Negl Trop Dis 9, e0004139 (2015).
40. Acharya, D. et al. Interleukin-17A Promotes CD8+ T Cell Cytotoxicity To Facilitate West Nile Virus Clearance. J Virol 91 (2017).
41. Bai, F. et al. IL-10 signaling blockade controls murine West Nile virus infection. PLoS Pathog 5, e1000610 (2009).
42. Shan, C. et al. A live-attenuated Zika virus vaccine candidate induces sterilizing immunity in mouse models.
43. Town, T. et al. Toll-like receptor 7 mitigates lethal West Nile encephalitis via interleukin 23-dependent immune cell infiltration and homing. Immunity 30, 242-253 (2009).
44. Abbink, P. et al. Protective efficacy of multiple vaccine platforms against Zika virus challenge in rhesus monkeys. Science 353, 1129-1132 (2016).
45. Sumathy, K. et al. Protective efficacy of Zika vaccine in AG129 mouse model. Sci Rep 7, 46375 (2017).
46. Xu, K. et al. Recombinant Chimpanzee Adenovirus Vaccine AdC7-M/E Protects against Zika Virus Infection and Testis Damage. J Virol 92 (2018).
47. Tebas, P. et al. Safety and Immunogenicity of an Anti-Zika Virus DNA Vaccine—Preliminary Report. N Engl J Med (2017).
48. Dowd, K. A. et al. Rapid development of a DNA vaccine for Zika virus. Science 354, 237-240 (2016).
49. Richner, J. M. et al. Modified mRNA Vaccines Protect against Zika Virus Infection. Cell 168, 1114-1125 e1110 (2017).
50. Yang, M., Sun, H., Lai, H., Hurtado, J. & Chen, Q. Plant-produced Zika virus envelope protein elicits neutralizing immune responses that correlate with protective immunity against Zika virus in mice. Plant Biotechnol J 16, 572-580 (2018).
51. Li, X. F. et al. Development of a chimeric Zika vaccine using a licensed live-attenuated flavivirus vaccine as backbone. Nat Commun 9, 673 (2018).
52. Poland, G. A., Ovsyannikova, I. G. & Kennedy, R. B. Zika Vaccine Development: Current Status. Mayo Clin Proc 94, 2572-2586 (2019).
53. Brisse, M., Vrba, S. M., Kirk, N., Liang, Y. & Ly, H. Emerging Concepts and Technologies in Vaccine Development. Front Immunol 11, 583077 (2020).
54. Collins, M. H. & Metz, S. W. Progress and Works in Progress: Update on Flavivirus Vaccine Development. Clin Ther 39, 1519-1536 (2017).
55. Lee, J., Arun Kumar, S., Jhan, Y. Y. & Bishop, C. J. Engineering DNA vaccines against infectious diseases.
56. Liu, T., Liang, Y. & Huang, L. Development and Delivery Systems of mRNA Vaccines.
57. Blaney, J. E., Jr. et al. Dengue virus type 3 vaccine candidates generated by introduction of deletions in the 3′ untranslated region (3′-UTR) or by exchange of the DENV-3 3′-UTR with that of DENV-4. Vaccine 26, 817-828 (2008).
58. Shan, C. et al. A single-dose live-attenuated vaccine prevents Zika virus pregnancy transmission and testis damage. Nat Commun 8, 676 (2017).
59. Ramanathan, M. P. et al. Host cell killing by the West Nile Virus NS2B-NS3 proteolytic complex: NS3 alone is sufficient to recruit caspase-8-based apoptotic pathway. Virology 345, 56-72 (2006).
60. Xing, H. et al. Zika NS2B is a crucial factor recruiting NS3 to the ER and activating its protease activity. Virus Research 275, 197793 (2020).
61. Bowen, J. R. et al. Zika Virus Antagonizes Type I Interferon Responses during Infection of Human Dendritic Cells. PLoS Pathog 13, e1006164 (2017).
62. Grant, A. et al. Zika Virus Targets Human STAT2 to Inhibit Type I Interferon Signaling. Cell Host Microbe 19, 882-890 (2016).
63. Parisien, J. P., Lenoir, J. J., Alvarado, G. & Horvath, C. M. The Human STAT2 Coiled-Coil Domain Contains a Degron for Zika Virus Interferon Evasion. J Virol 96, e0130121 (2022).
64. Gambino, F., Jr. et al. A vaccine inducing solely cytotoxic T lymphocytes fully prevents Zika virus infection and fetal damage. Cell Rep 35, 109107 (2021).
65. Yang, Y. et al. A cDNA Clone-Launched Platform for High-Yield Production of Inactivated Zika Vaccine.
66. Shan, C. et al. An Infectious cDNA Clone of Zika Virus to Study Viral Virulence, Mosquito Transmission, and Antiviral Inhibitors.
67. Acharya, D. et al. An ultrasensitive electrogenerated chemiluminescence-based immunoassay for specific detection of Zika virus. Sci Rep 6, 32227 (2016).
68. Neupane, B. et al. Interleukin-17A Facilitates Chikungunya Virus Infection by Inhibiting IFNalpha2 Expression. Front Immunol 11, 588382 (2020).
69. Bai, F. et al. Use of RNA interference to prevent lethal murine west nile virus infection. J Infect Dis 191, 1148-1154 (2005).
70. Paul, A. M. et al. Delivery of antiviral small interfering RNA with gold nanoparticles inhibits dengue virus infection in vitro. J Gen Virol 95, 1712-1722 (2014).
71. Cacciabue, M., Curria, A. & Gismondi, M. I. ViralPlaque: a Fiji macro for automated assessment of viral plaque statistics. PeerJ 7, e7729 (2019).
72. Sun, H. et al. Antibody-Dependent Enhancement Activity of a Plant-Made Vaccine against West Nile Virus.
73. Andrews, R. J., Roche, J. & Moss, W. N. ScanFold: an approach for genome-wide discovery of local RNA structural elements-applications to Zika virus and HIV. PeerJ 6, e6136 (2018).
74. Huang, F. (2003). Efficient incorporation of CoA, NAD and FAD into RNA by in vitro transcription. Nucleic Acids Res 31, e8.
75. Coleman, T. M., Wang, G., and Huang, F. (2004). Superior 5′ homogeneity of RNA from ATP-initiated transcription under the T7 phi 2.5 promoter. Nucleic Acids Res 32, e14.
76. He, Y., Zhou, Y., Siddiqui, P., and Jiang, S. (2004). Inactivated SARS-CoV vaccine elicits high titers of spike protein-specific antibodies that block receptor binding and virus entry. Biochem Biophys Res Commun 325, 445-452.
77. He, Y., Li, J., Li, W., Lustigman, S., Farzan, M., and Jiang, S. (2006). Cross-neutralization of human and palm civet severe acute respiratory syndrome coronaviruses by antibodies targeting the receptor-binding domain of spike protein. J Immunol 176, 6085-6092.
78. He, Y., Lu, H., Siddiqui, P., Zhou, Y., and Jiang, S. (2005). Receptor-binding domain of severe acute respiratory syndrome coronavirus spike protein contains multiple conformation-dependent epitopes that induce highly potent neutralizing antibodies. J Immunol 174, 4908-4915.
79. Zhi, Y., Kobinger, G. P., Jordan, H., Suchma, K., Weiss, S. R., Shen, H., Schumer, G., Gao, G., Boyer, J. L., Crystal, R. G., et al. (2005). Identification of murine CD8 T cell epitopes in codon-optimized SARS-associated coronavirus spike protein. Virology 335, 34-45.
80. Zakhartchouk, A. N., Sharon, C., Satkunarajah, M., Auperin, T., Viswanathan, S., Mutwiri, G., Petric, M., See, R. H., Brunham, R. C., Finlay, B. B., et al. (2007). Immunogenicity of a receptor-binding domain of SARS coronavirus spike protein in mice: implications for a subunit vaccine. Vaccine 25, 136-143.
81. Du, L., Zhao, G., Lin, Y., Sui, H., Chan, C., Ma, S., He, Y., Jiang, S., Wu, C., Yuen, K. Y., et al. (2008). Intranasal vaccination of recombinant adeno-associated virus encoding receptor-binding domain of severe acute respiratory syndrome coronavirus (SARS-CoV) spike protein induces strong mucosal immune responses and provides long-term protection against SARS-CoV infection. J Immunol 180, 948-956.
82. Du, L., He, Y., Zhou, Y., Liu, S., Zheng, B. J., and Jiang, S. (2009). The spike protein of SARS-CoV—a target for vaccine and therapeutic development. Nat Rev Microbiol 7, 226-236.
83. Chen, Z., and John Wherry, E. (2020). T cell responses in patients with COVID-19. Nat Rev Immunol 20, 529-536.
84. Poh, C. M., Carissimo, G., Wang, B., Amrun, S. N., Lee, C. Y., Chee, R. S., Fong, S. W., Yeo, N. K., Lee, W. H., Torres-Ruesta, A., et al. (2020). Two linear epitopes on the SARS-CoV-2 spike protein that elicit neutralising antibodies in COVID-19 patients. Nat Commun 11, 2806.
85. Ravichandran, S., Coyle, E. M., Klenow, L., Tang, J., Grubbs, G., Liu, S., Wang, T., Golding, H., and Khurana, S. (2020). Antibody signature induced by SARS-CoV-2 spike protein immunogens in rabbits. Sci Transl Med 12.
86. Keng, C. T., Zhang, A., Shen, S., Lip, K. M., Fielding, B. C., Tan, T. H., Chou, C. F., Loh, C. B., Wang, S., Fu, J., et al. (2005). Amino acids 1055 to 1192 in the S2 region of severe acute respiratory syndrome coronavirus S protein induce neutralizing antibodies: implications for the development of vaccines and antiviral agents. J Virol 79, 3289-3296.
87. Okada, M., Okuno, Y., Hashimoto, S., Kita, Y., Kanamaru, N., Nishida, Y., Tsunai, Y., Inoue, R., Nakatani, H., Fukamizu, R., et al. (2007). Development of vaccines and passive immunotherapy against SARS coronavirus using SCID-PBL/hu mouse models. Vaccine 25, 3038-3040.
88. Wang, Z., Yuan, Z., Matsumoto, M., Hengge, U. R., and Chang, Y. F. (2005). Immune responses with DNA vaccines encoded different gene fragments of severe acute respiratory syndrome coronavirus in BALB/c mice. Biochem Biophys Res Commun 327, 130-135.
89. Yang, L., Peng, H., Zhu, Z., Li, G., Huang, Z., Zhao, Z., Koup, R. A., Bailer, R. T., and Wu, C. (2007). Persistent memory CD4+ and CD8+ T-cell responses in recovered severe acute respiratory syndrome (SARS) patients to SARS coronavirus M antigen. J Gen Virol 88, 2740-2748.
90. Boots, A. M., Kusters, J. G., van Noort, J. M., Zwaagstra, K. A., Rijke, E., van der Zeijst, B. A., and Hensen, E. J. (1991). Localization of a T-cell epitope within the nucleocapsid protein of avian coronavirus. Immunology 74, 8-13.
91. Enjuanes, L., Smerdou, C., Castilla, J., Anton, I. M., Torres, J. M., Sola, I., Golvano, J., Sanchez, J. M., and Pintado, B. (1995). Development of protection against coronavirus induced diseases. A review. Adv Exp Med Biol 380, 197-211.
92. Gao, W., Tamin, A., Soloff, A., D'Aiuto, L., Nwanegbo, E., Robbins, P. D., Bellini, W. J., Barratt-Boyes, S., and Gambotto, A. (2003). Effects of a SARS-associated coronavirus vaccine in monkeys. Lancet 362, 1895-1896.
93. Kim, T. W., Lee, J. H., Hung, C. F., Peng, S., Roden, R., Wang, M. C., Viscidi, R., Tsai, Y. C., He, L., Chen, P. J., et al. (2004). Generation and characterization of DNA vaccines targeting the nucleocapsid protein of severe acute respiratory syndrome coronavirus. J Virol 78, 4638-4645.
94. Schott, J. W., Morgan, M., Galla, M., and Schambach, A. (2016). Viral and Synthetic RNA Vector Technologies and Applications. Mol Ther 24, 1513-1527.
95. Pardi, N., Hogan, M. J., Porter, F. W., and Weissman, D. (2018). mRNA vaccines—a new era in vaccinology. Nat Rev Drug Discov 17, 261-279.
96. Jackson, N. A. C., Kester, K. E., Casimiro, D., Gurunathan, S., and DeRosa, F. (2020). The promise of mRNA vaccines: a biotech and industrial perspective. NPJ Vaccines 5, 11.
97. Zhang, C., Maruggi, G., Shan, H., and Li, J. (2019). Advances in mRNA Vaccines for Infectious Diseases. Front Immunol 10, 594.
98. Martinon, F., Krishnan, S., Lenzen, G., Magne, R., Gomard, E., Guillet, J. G., Levy, J. P., and Meulien, P. (1993). Induction of virus-specific cytotoxic T lymphocytes in vivo by liposome-entrapped mRNA. Eur J Immunol 23, 1719-1722.
99. Vogel, A. B., Lambert, L., Kinnear, E., Busse, D., Erbar, S., Reuter, K. C., Wicke, L., Perkovic, M., Beissert, T., Haas, H., et al. (2018). Self-Amplifying RNA Vaccines Give Equivalent Protection against Influenza to mRNA Vaccines but at Much Lower Doses. Mol Ther 26, 446-455.
100. Chen, N., Xia, P., Li, S., Zhang, T., Wang, T. T., and Zhu, J. (2017). RNA sensors of the innate immune system and their detection of pathogens. IUBMB Life 69, 297-304.
101. Berglund, P., Quesada-Rolander, M., Putkonen, P., Biberfeld, G., Thorstensson, R., and Liljestrom, P. (1997). Outcome of immunization of cynomolgus monkeys with recombinant Semliki Forest virus encoding human immunodeficiency virus type 1 envelope protein and challenge with a high dose of SHIV-4 virus. AIDS Res Hum Retroviruses 13, 1487-1495.
102. Pushko, P., Parker, M., Ludwig, G. V., Davis, N. L., Johnston, R. E., and Smith, J. F. (1997). Replicon-helper systems from attenuated Venezuelan equine encephalitis virus: expression of heterologous genes in vitro and immunization against heterologous pathogens in vivo. Virology 239, 389-401.
103. Hariharan, M. J., Driver, D. A., Townsend, K., Brumm, D., Polo, J. M., Belli, B. A., Catton, D. J., Hsu, D., Mittelstaedt, D., McCormack, J. E., et al. (1998). DNA immunization against herpes simplex virus: enhanced efficacy using a Sindbis virus-based vector. J Virol 72, 950-958.
104. Cheng, W. F., Hung, C. H., Chai, C. Y., Hsu, K. F., He, L., Ling, M., and Wu, T. C. (2001). Enhancement of sindbis virus self-replicating RNA vaccine potency by linkage of herpes simplex virus type 1 VP22 protein to antigen. J Virol 75, 2368-2376.
105. Perri, S., Greer, C. E., Thudium, K., Doe, B., Legg, H., Liu, H., Romero, R. E., Tang, Z., Bin, Q., Dubensky, T. W., Jr., et al. (2003). An alphavirus replicon particle chimera derived from Venezuelan equine encephalitis and sindbis viruses is a potent gene-based vaccine delivery vector. J Virol 77, 10394-10403.
106. Ljungberg, K., and Liljestrom, P. (2015). Self-replicating alphavirus RNA vaccines. Expert Rev Vaccines 14, 177-194.
107. Pijlman, G. P., Suhrbier, A., and Khromykh, A. A. (2006). Kunjin virus replicons: an RNA-based, non-cytopathic viral vector system for protein production, vaccine and gene therapy applications. Expert Opin Biol Ther 6, 135-145.
108. McCullough, K. C., Bassi, I., Milona, P., Suter, R., Thomann-Harwood, L., Englezou, P., Demoulins, T., and Ruggli, N. (2014). Self-replicating Replicon-RNA Delivery to Dendritic Cells by Chitosan-nanoparticles for Translation In Vitro and In Vivo. Mol Ther Nucleic Acids 3, e173.
109. Englezou, P. C., Sapet, C., Demoulins, T., Milona, P., Ebensen, T., Schulze, K., Guzman, C. A., Poulhes, F., Zelphati, O., Ruggli, N., et al. (2018). Self-Amplifying Replicon RNA Delivery to Dendritic Cells by Cationic Lipids. Mol Ther Nucleic Acids 12, 118-134.
110. Pugachev, K. V., Guirakhoo, F., Ocran, S. W., Mitchell, F., Parsons, M., Penal, C., Girakhoo, S., Pougatcheva, S. O., Arroyo, J., Trent, D. W., et al. (2004). High fidelity of yellow fever virus RNA polymerase. J Virol 78, 1032-1038.
111. King, N. J., and Kesson, A. M. (1988). Interferon-independent increases in class I major histocompatibility complex antigen expression follow flavivirus infection. J Gen Virol 69 (Pt 10), 2535-2543.
112. Mullbacher, A., and Lobigs, M. (1995). Up-regulation of MHC class I by flavivirus-induced peptide translocation into the endoplasmic reticulum. Immunity 3, 207-214.
113. Li, W., Moore, M. J., Vasilieva, N., Sui, J., Wong, S. K., Berne, M. A., Somasundaran, M., Sullivan, J. L., Luzuriaga, K., Greenough, T. C., et al. (2003). Angiotensin-converting enzyme 2 is a functional receptor for the SARS coronavirus. Nature 426, 450-454.
114. Masters, P. S. (2006). The molecular biology of coronaviruses. Adv Virus Res 66, 193-292.
115. Raj, V. S., Mou, H., Smits, S. L., Dekkers, D. H., Muller, M. A., Dijkman, R., Muth, D., Demmers, J. A., Zaki, A., Fouchier, R. A., et al. (2013). Dipeptidyl peptidase 4 is a functional receptor for the emerging human coronavirus—EMC. Nature 495, 251-254.
116. Walls, A. C., Park, Y. J., Tortorici, M. A., Wall, A., McGuire, A. T., and Veesler, D. (2020). Structure, Function, and Antigenicity of the SARS-CoV-2 Spike Glycoprotein. Cell.
117. Hoffmann, M., Kleine-Weber, H., Schroeder, S., Kruger, N., Herrder, T., Erichsen, S., Schiergens, T. S., Herrler, G., Wu, N. H., Nitsche, A., et al. (2020). SARS-CoV-2 Cell Entry Depends on ACE2 and TMPRSS2 and Is Blocked by a Clinically Proven Protease Inhibitor. Cell 181, 271-280 e278.
118. Wang, S. F., Tseng, S. P., Yen, C. H., Yang, J. Y., Tsao, C. H., Shen, C. W., Chen, K. H., Liu, F. T., Liu, W. T., Chen, Y. M., et al. (2014). Antibody-dependent SARS coronavirus infection is mediated by antibodies against spike proteins. Biochem Biophys Res Commun 451, 208-214.
119. Yip, M. S., Leung, N. H., Cheung, C. Y., Li, P. H., Lee, H. H., Daeron, M., Peiris, J. S., Bruzzone, R., and Jaume, M. (2014). Antibody-dependent infection of human macrophages by severe acute respiratory syndrome coronavirus. Virol J 11, 82.
120. Wang, Q., Zhang, L., Kuwahara, K., Li, L., Liu, Z., Li, T., Zhu, H., Liu, J., Xu, Y., Xie, J., et al. (2016). Immunodominant SARS Coronavirus Epitopes in Humans Elicited both Enhancing and Neutralizing Effects on Infection in Non-human Primates. ACS Infect Dis 2, 361-376.
121. Tetro, J. A. (2020). Is COVID-19 receiving ADE from other coronaviruses?Microbes Infect 22, 72-73.
122. Tilocca, B., Soggiu, A., Musella, V., Britti, D., Sanguinetti, M., Urbani, A., and Roncada, P. (2020). Molecular basis of COVID-19 relationships in different species: a one health perspective. Microbes Infect.
123. Wan, Y., Shang, J., Sun, S., Tai, W., Chen, J., Geng, Q., He, L., Chen, Y., Wu, J., Shi, Z., et al. (2020). Molecular Mechanism for Antibody-Dependent Enhancement of Coronavirus Entry. J Virol 94.
124. Huang, F., Spangler, J. R., and Huang, A. Y. (2017). In vivo cloning of up to 16 kb plasmids in E. coli is as simple as PCR. PLoS One 12, e0183974.
125. Sapkota, K., and Huang, F. (2018). Efficient one-pot enzymatic synthesis of dephospho coenzyme A. Bioorg Chem 76, 23-27.
126. Dhakal, S., Sapkota, K., Huang, F., and Rangachari, V. (2020). Cloning, expression and purification of the low-complexity region of RanBP9 protein. Protein Expr Purif 172, 105630.
127. Peiris, J. S., Guan, Y., and Yuen, K. Y. (2004). Severe acute respiratory syndrome. Nat Med 10, S88-97.
128. Li, W., Zhang, C., Sui, J., Kuhn, J. H., Moore, M. J., Luo, S., Wong, S. K., Huang, I. C., Xu, K., Vasilieva, N., et al. (2005). Receptor and viral determinants of SARS-coronavirus adaptation to human ACE2. EMBO J 24, 1634-1643.
129. Letko, M., Marzi, A., and Munster, V. (2020). Functional assessment of cell entry and receptor usage for SARS-CoV-2 and other lineage B betacoronaviruses. Nat Microbiol 5, 562-569.
130. Calam, J., Yiangou, Y., Chrysanthou, B., Paul, G., Mehta, A., and Bloom, S. R. (1988). Peptidehistidine methionine (PHM) increases ileostomy output. Regul Pept 23, 57-62.
131. Yang, X. H., Deng, W., Tong, Z., Liu, Y. X., Zhang, L. F., Zhu, H., Gao, H., Huang, L., Liu, Y. L., Ma, C. M., et al. (2007). Mice transgenic for human angiotensin-converting enzyme 2 provide a model for SARS coronavirus infection. Comp Med 57, 450-459.
132. McCray, P. B., Jr., Pewe, L., Wohlford-Lenane, C., Hickey, M., Manzel, L., Shi, L., Netland, J., Jia, H. P., Halabi, C., Sigmund, C. D., et al. (2007). Lethal infection of K18-hACE2 mice infected with severe acute respiratory syndrome coronavirus. J Virol 81, 813-821.
133. Bao, L., Gao, H., Deng, W., Lv, Q., Yu, H., Liu, M., Yu, P., Liu, J., Qu, Y., Gong, S., et al. (2020). Transmission of SARS-CoV-2 via close contact and respiratory droplets among hACE2 mice. J Infect Dis.
134. Cao, Y., Su, B., Guo, X., Sun, W., Deng, Y., Bao, L., Zhu, Q., Zhang, X., Zheng, Y., Geng, C., et al. (2020). Potent neutralizing antibodies against SARS-CoV-2 identified by high-throughput single-cell sequencing of convalescent patients' B cells. Cell.
135. Jiang, R. D., Liu, M. Q., Chen, Y., Shan, C., Zhou, Y. W., Shen, X. R., Li, Q., Zhang, L., Zhu, Y., Si, H. R., et al. (2020). Pathogenesis of SARS-CoV-2 in Transgenic Mice Expressing Human Angiotensin-Converting Enzyme 2. Cell 182, 50-58 e58.
136. Winkler, E. S., Bailey, A. L., Kafai, N. M., Nair, S., McCune, B. T., Yu, J., Fox, J. M., Chen, R. E., Earnest, J. T., Keeler, S. P., et al. (2020). SARS-CoV-2 infection of human ACE2-transgenic mice causes severe lung inflammation and impaired function. Nat Immunol 21, 1327-1335.
137. Liu, M. A. (2019). A Comparison of Plasmid DNA and mRNA as Vaccine Technologies. Vaccines (Basel) 7.
138. Leitner, W. W., Ying, H., and Restifo, N. P. (1999). DNA and RNA-based vaccines: principles, progress and prospects. Vaccine 18, 765-777.
139. Kowalski, P. S., Rudra, A., Miao, L., and Anderson, D. G. (2019). Delivering the Messenger: Advances in Technologies for Therapeutic mRNA Delivery. Mol Ther 27, 710-728.
140. Gomez-Aguado, I., Rodriguez-Castejon, J., Vicente-Pascual, M., Rodriguez-Gascon, A., Solinis, M. A., and Del Pozo-Rodriguez, A. (2020). Nanomedicines to Deliver mRNA: State of the Art and Future Perspectives. Nanomaterials (Basel) 10.
141. Lundstrom, K. (2015). Alphaviruses in gene therapy. Viruses 7, 2321-2333.
142. Berglund, P., Smerdou, C., Fleeton, M. N., Tubulekas, I., and Liljestrom, P. (1998). Enhancing immune responses using suicidal DNA vaccines. Nat Biotechnol 16, 562-565.
143. Geall, A. J., Verma, A., Otten, G. R., Shaw, C. A., Hekele, A., Banerjee, K., Cu, Y., Beard, C. W., Brito, L. A., Krucker, T., et al. (2012). Nonviral delivery of self-amplifying RNA vaccines. Proc Natl Acad Sci USA 109, 14604-14609.
144. Li, Y., Teague, B., Zhang, Y., Su, Z., Porter, E., Dobosh, B., Wagner, T., Irvine, D. J., and Weiss, R. (2019). In vitro evolution of enhanced RNA replicons for immunotherapy. Sci Rep 9, 6932.
145. Hekele, A., Bertholet, S., Archer, J., Gibson, D. G., Palladino, G., Brito, L. A., Otten, G. R., Brazzoli, M., Buccato, S., Bonci, A., et al. (2013). Rapidly produced SAM((R)) vaccine against H7N9 influenza is immunogenic in mice. Emerg Microbes Infect 2, e52.
146. Pang, X., Zhang, M., and Dayton, A. I. (2001). Development of Dengue virus type 2 replicons capable of prolonged expression in host cells. BMC Microbiol 1, 18.
147. Shi, P. Y., Tilgner, M., and Lo, M. K. (2002). Construction and characterization of subgenomic replicons of New York strain of West Nile virus. Virology 296, 219-233.
148. Scholle, F., Girard, Y. A., Zhao, Q., Higgs, S., and Mason, P. W. (2004). trans-Packaged West Nile virus-like particles: infectious properties in vitro and in infected mosquito vectors. J Virol 78, 11605-11614.
149. Jones, C. T., Patkar, C. G., and Kuhn, R. J. (2005). Construction and applications of yellow fever virus replicons. Virology 331, 247-259.
150. Jones, M., Davidson, A., Hibbert, L., Gruenwald, P., Schlaak, J., Ball, S., Foster, G. R., and Jacobs, M. (2005). Dengue virus inhibits alpha interferon signaling by reducing STAT2 expression. J Virol 79, 5414-5420.
151. Xie, X., Zou, J., Shan, C., Yang, Y., Kum, D. B., Dallmeier, K., Neyts, J., and Shi, P. Y. (2016). Zika Virus Replicons for Drug Discovery. EBioMedicine 12, 156-160.
152. Gallagher, T. M., and Buchmeier, M. J. (2001). Coronavirus spike proteins in viral entry and pathogenesis. Virology 279, 371-374.
153. Lu, G., Hu, Y., Wang, Q., Qi, J., Gao, F., Li, Y., Zhang, Y., Zhang, W., Yuan, Y., Bao, J., et al. (2013). Molecular basis of binding between novel human coronavirus MERS-CoV and its receptor CD26. Nature 500, 227-231.
154. Wang, Y. D., Sin, W. Y., Xu, G. B., Yang, H. H., Wong, T. Y., Pang, X. W., He, X. Y., Zhang, H. G., Ng, J. N., Cheng, C. S., et al. (2004). T-cell epitopes in severe acute respiratory syndrome (SARS) coronavirus spike protein elicit a specific T-cell immune response in patients who recover from SARS. J Virol 78, 5612-5618.
155. Nie, Y., Wang, G., Shi, X., Zhang, H., Qiu, Y., He, Z., Wang, W., Lian, G., Yin, X., Du, L., et al. (2004). Neutralizing antibodies in patients with severe acute respiratory syndrome-associated coronavirus infection. J Infect Dis 190, 1119-1126.
156. Zhong, X., Yang, H., Guo, Z. F., Sin, W. Y., Chen, W., Xu, J., Fu, L., Wu, J., Mak, C. K., Cheng, C. S., et al. (2005). B-cell responses in patients who have recovered from severe acute respiratory syndrome target a dominant site in the S2 domain of the surface spike glycoprotein. J Virol 79, 3401-3408.
157. Li, C. K., Wu, H., Yan, H., Ma, S., Wang, L., Zhang, M., Tang, X., Temperton, N. J., Weiss, R. A., Brenchley, J. M., et al. (2008). T cell responses to whole SARS coronavirus in humans. J Immunol 181, 5490-5500.
158. Zhao, J., Alshukairi, A. N., Baharoon, S. A., Ahmed, W. A., Bokhari, A. A., Nehdi, A. M., Layqah, L. A., Alghamdi, M. G., Al Gethamy, M. M., Dada, A. M., et al. (2017). Recovery from the Middle East respiratory syndrome is associated with antibody and T-cell responses. Sci Immunol 2.
159. Jaume, M., Yip, M. S., Cheung, C. Y., Leung, H. L., Li, P. H., Kien, F., Dutry, I., Callendret, B., Escriou, N., Altmeyer, R., et al. (2011). Anti-severe acute respiratory syndrome coronavirus spike antibodies trigger infection of human immune cells via a pH- and cysteine protease-independent FcgammaR pathway. J Virol 85, 10582-10597.
160. Jaume, M., Yip, M. S., Kam, Y. W., Cheung, C. Y., Kien, F., Roberts, A., Li, P. H., Dutry, I., Escriou, N., Daeron, M., et al. (2012). SARS CoV subunit vaccine: antibody-mediated neutralisation and enhancement. Hong Kong Med J 18 Suppl 2, 31-36.
161. Walls, A. C., Xiong, X., Park, Y. J., Tortorici, M. A., Snijder, J., Quispe, J., Cameroni, E., Gopal, R., Dai, M., Lanzavecchia, A., et al. (2019). Unexpected Receptor Functional Mimicry Elucidates Activation of Coronavirus Fusion. Cell 176, 1026-1039 e1015.
162. Weingartd, H., Czub, M., Czub, S., Neufeld, J., Marszal, P., Gren, J., Smith, G., Jones, S., Proulx, R., Deschambault, Y., et al. (2004). Immunization with modified vaccinia virus Ankara-based recombinant vaccine against severe acute respiratory syndrome is associated with enhanced hepatitis in ferrets. J Virol 78, 12672-12676.
163. Czub, M., Weingartd, H., Czub, S., He, R., and Cao, J. (2005). Evaluation of modified vaccinia virus Ankara based recombinant SARS vaccine in ferrets. Vaccine 23, 2273-2279.
164. Kam, Y. W., Kien, F., Roberts, A., Cheung, Y. C., Lamirande, E. W., Vogel, L., Chu, S. L., Tse, J., Guarner, J., Zaki, S. R., et al. (2007). Antibodies against trimeric S glycoprotein protect hamsters against SARS-CoV challenge despite their capacity to mediate FcgammaRII-dependent entry into B cells in vitro. Vaccine 25, 729-740.
165. Du, L., Tai, W., Zhou, Y., and Jiang, S. (2016). Vaccines for the prevention against the threat of MERS-CoV. Expert Rev Vaccines 15, 1123-1134.
166. Jiang, S., Hillyer, C., and Du, L. (2020). Neutralizing Antibodies against SARS-CoV-2 and Other Human Coronaviruses: (Trends in Immunology 41, 355-359; 2020). Trends Immunol.
167. Thomson, E. C.a.a. (2020). The circulating SARS-CoV-2 spike variant N439K maintains fitness while evading antibody-mediated immunity. https://www.biorxiv.org/content/10.1101/2020.11.04.355842v1.full.pdf.
168. ampgoo.com (2020). A public health nightmare—why a new strain of coronavirus could hamper vaccine hopes. https//www.ampgoo.com/a-public-health-nightmare-why-a-new-strain-of-coronavirus-could-hamper-vaccine-hops.
169. Du, L., Tai, W., Yang, Y., Zhao, G., Zhu, Q., Sun, S., Liu, C., Tao, X., Tseng, C. K., Perlman, S., et al. (2016). Introduction of neutralizing immunogenicity index to the rational design of MERS coronavirus subunit vaccines. Nat Commun 7, 13473.
170. Okba, N. M., Raj, V. S., and Haagmans, B. L. (2017). Middle East respiratory syndrome coronavirus vaccines: current status and novel approaches. Curr Opin Virol 23, 49-58.
171. Xu, X., and Gao, X. (2004). Immunological responses against SARS-coronavirus infection in humans. CellMol Immunol 1, 119-122.
172. Li, T., Xie, J., He, Y., Fan, H., Baril, L., Qiu, Z., Han, Y., Xu, W., Zhang, W., You, H., et al. (2006). Long-term persistence of robust antibody and cytotoxic T cell responses in recovered patients infected with SARS coronavirus. PLoS One 1, e24.
173. Yang, J., Wang, W., Chen, Z., Lu, S., Yang, F., Bi, Z., Bao, L., Mo, F., Li, X., Huang, Y., et al. (2020). A vaccine targeting the RBD of the S protein of SARS-CoV-2 induces protective immunity. Nature 586, 572-577.
174. Hurlburt, N. K., Seydoux, E., Wan, Y. H., Edara, V. V., Stuart, A. B., Feng, J., Suthar, M. S., McGuire, A. T., Stamatatos, L., and Pancera, M. (2020). Structural basis for potent neutralization of SARS-CoV-2 and role of antibody affinity maturation. Nat Commun 11, 5413.
175. Walls, A. C., Fiala, B., Schafer, A., Wrenn, S., Pham, M. N., Murphy, M., Tse, L. V., Shehata, L., O'Connor, M. A., Chen, C., et al. (2020). Elicitation of Potent Neutralizing Antibody Responses by Designed Protein Nanoparticle Vaccines for SARS-CoV-2. Cell.
176. Zhu, Y., Yu, D., Yan, H., Chong, H., and He, Y. (2020). Design of Potent Membrane Fusion Inhibitors against SARS-CoV-2, an Emerging Coronavirus with High Fusogenic Activity. J Virol 94.
177. Rabets, A.a.a. (2020). Development of antibodies to pan-coronavirus spike peptides in convalescent COVID-19 patients. https://www.medrxiv.org/content/10.1101/2020.08.20.20178566v1.
178. Liu, S., Xiao, G., Chen, Y., He, Y., Niu, J., Escalante, C. R., Xiong, H., Farmar, J., Debnath, A. K., Tien, P., et al. (2004). Interaction between heptad repeat 1 and 2 regions in spike protein of SARS-associated coronavirus: implications for virus fusogenic mechanism and identification of fusion inhibitors. Lancet 363, 938-947.
179. Khairkhah, N., Aghasadeghi, M. R., Namvar, A., and Bolhassani, A. (2020). Design of novel multiepitope constructs-based peptide vaccine against the structural S, N and M proteins of human COVID-19 using immunoinformatics analysis. PLoS One 15, e0240577.
180. Tan, Y. J., Goh, P. Y., Fielding, B. C., Shen, S., Chou, C. F., Fu, J. L., Leong, H. N., Leo, Y. S., Ooi, E. E., Ling, A. E., et al. (2004). Profiles of antibody responses against severe acute respiratory syndrome coronavirus recombinant proteins and their potential use as diagnostic markers. Clin Diagn Lab Immunol 11, 362-371.
181. Wu, H. S., Hsieh, Y. C., Su, I. J., Lin, T. H., Chiu, S. C., Hsu, Y. F., Lin, J. H., Wang, M. C., Chen, J. Y., Hsiao, P. W., et al. (2004). Early detection of antibodies against various structural proteins of the SARS-associated coronavirus in SARS patients. J Biomed Sci 11, 117-126.
182. Meyer, B., Drosten, C., and Muller, M. A. (2014). Serological assays for emerging coronaviruses: challenges and pitfalls. Virus Res 194, 175-183.
183. To, K. K., Tsang, O. T., Leung, W. S., Tam, A. R., Wu, T. C., Lung, D. C., Yip, C. C., Cai, J. P., Chan, J. M., Chik, T. S., et al. (2020). Temporal profiles of viral load in posterior oropharyngeal saliva samples and serum antibody responses during infection by SARS-CoV-2: an observational cohort study. Lancet Infect Dis 20, 565-574.
184. Zhang, Y., Xu, J., Jia, R., Yi, C., Gu, W., Liu, P., Dong, X., Zhou, H., Shang, B., Cheng, S., et al. (2020). Protective humoral immunity in SARS-CoV-2 infected pediatric patients. Cell Mol Immunol 17, 768-770.
185. Wang, R., Teng, C., Spangler, J., Wang, J., Huang, F., and Guo, Y. L. (2014). Mouse embryonic stem cells have underdeveloped antiviral mechanisms that can be exploited for the development of mRNA-mediated gene expression strategy. Stem Cells Dev 23, 594-604.
186. Baker, C., Liu, Y., Zou, J., Muruato, A., Xie, X., and Shi, P. Y. (2020). Identifying optimal capsid duplication length for the stability of reporter flaviviruses. Emerg Microbes Infect 9, 2256-2265.
187. Biddlecome, A., Habte, H. H., McGrath, K. M., Sambanthamoorthy, S., Wurm, M., Sykora, M. M., Knobler, C. M., Lorenz, I. C., Lasaro, M., Elbers, K., et al. (2019). Delivery of self-amplifying RNA vaccines in in vitro reconstituted virus-like particles. PLoS One 14, e0215031.
188. Bai, F., Kong, K. F., Dai, J., Qian, F., Zhang, L., Brown, C. R., Fikrig, E., and Montgomery, R. R. (2010). A paradoxical role for neutrophils in the pathogenesis of West Nile virus. J Infect Dis 202, 1804-1812.
189. D'Angelo, W., Acharya, D., Wang, R., Wang, J., Gurung, C., Chen, B., Bai, F., and Guo, Y. L. (2016). Development of Antiviral Innate Immunity During In Vitro Differentiation of Mouse Embryonic Stem Cells. Stem Cells Dev 25, 648-659.
190. Wang, R., Wang, J., Paul, A. M., Acharya, D., Bai, F., Huang, F., and Guo, Y. L. (2013). Mouse embryonic stem cells are deficient in type I interferon expression in response to viral infections and double-stranded RNA. J Biol Chem 288, 15926-15936.
191. Wang, R., Wang, J., Acharya, D., Paul, A. M., Bai, F., Huang, F., and Guo, Y. L. (2014). Antiviral responses in mouse embryonic stem cells: differential development of cellular mechanisms in type I interferon production and response. J Biol Chem 289, 25186-25198.
192. Lundstrom, K. (2020). Nanoparticle-based delivery of self-amplifying RNA. Gene Ther.
193. Angel, M., and Yanik, M. F. (2010). Innate immune suppression enables frequent transfection with RNA encoding reprogramming proteins. PLoS One 5, e11756.
194. Bao, L., Deng, W., Huang, B., Gao, H., Liu, J., Ren, L., Wei, Q., Yu, P., Xu, Y., Qi, F., et al. (2020). The pathogenicity of SARS-CoV-2 in hACE2 transgenic mice. Nature 583, 830-833.
195. Lutz, C., Maher, L., Lee, C., and Kang, W. (2020). COVID-19 preclinical models: human angiotensin-converting enzyme 2 transgenic mice. Hum Genomics 14, 20.
196. Busnadiego, I., Fernbach, S., Pohl, M. O., Karakus, U., Huber, M., Trkola, A., Stertz, S., and Hale, B. G. (2020). Antiviral Activity of Type I, IL, and III Interferons Counterbalances ACE2 Inducibility and Restricts SARS-CoV-2. mBio 11.
197. Zhang, N. N., Li, X. F., Deng, Y. Q., Zhao, H., Huang, Y. J., Yang, G., Huang, W. J., Gao, P., Zhou, C., Zhang, R. R., et al. (2020). A Thermostable mRNA Vaccine against COVID-19. Cell 182, 1271-1283 e1216.
198. Smith, T. R. F., Patel, A., Ramos, S., Elwood, D., Zhu, X., Yan, J., Gary, E. N., Walker, S. N., Schultheis, K., Purwar, M., et al. (2020). Immunogenicity of a DNA vaccine candidate for COVID-19. Nat Commun 11, 2601.
199. Acharya, D., Wang, P., Paul, A. M., Dai, J., Gate, D., Lowery, J. E., Stokic, D. S., Leis, A. A., Flavell, R. A., Town, T., et al. (2017). Interleukin-17A Promotes CD8+ T Cell Cytotoxicity To Facilitate West Nile Virus Clearance. J Virol 91.
200. Paul, A. M., Acharya, D., Le, L., Wang, P., Stokic, D. S., Leis, A. A., Alexopoulou, L., Town, T., Flavell, R. A., Fikrig, E., et al. (2016). TLR8 Couples SOCS-1 and Restrains TLR7-Mediated Antiviral Immunity, Exacerbating West Nile Virus Infection in Mice. J Immunol 197, 4425-4435.
201. Bai, F., Town, T., Qian, F., Wang, P., Kamanaka, M., Connolly, T. M., Gate, D., Montgomery, R. R., Flavell, R. A., and Fikrig, E. (2009). IL-10 signaling blockade controls murine West Nile virus infection. PLoS Pathog 5, e1000610.
202. Town, T., Bai, F., Wang, T., Kaplan, A. T., Qian, F., Montgomery, R. R., Anderson, J. F., Flavell, R. A., and Fikrig, E. (2009). Toll-like receptor 7 mitigates lethal West Nile encephalitis via interleukin 23-dependent immune cell infiltration and homing. Immunity 30, 242-253.
203. Van Hoecke, L., Job, E. R., Saelens, X., and Roose, K. (2017). Bronchoalveolar Lavage of Murine Lungs to Analyze Inflammatory Cell Infiltration. J Vis Exp.
204. Zang, J., Gu, C., Zhou, B., Zhang, C., Yang, Y., Xu, S., Bai, L., Zhang, R., Deng, Q., Yuan, Z., et al. (2020). Immunization with the receptor-binding domain of SARS-CoV-2 elicits antibodies cross-neutralizing SARS-CoV-2 and SARS-CoV without antibody-dependent enhancement. Cell Discov. 6, 61.
205. Liu, X., Drelich, A., Li, W., Chen, C., Sun, Z., Shi, M., Adams, C., Mellors, J. W., Tseng, C. T., and Dimitrov, D. S. (2020). Enhanced elicitation of potent neutralizing antibodies by the SARS-CoV-2 spike receptor binding domain Fc fusion protein in mice. Vaccine 38, 7205-7212.
206. Dent, M., Hurtado, J., Paul, A. M., Sun, H., Lai, H., Yang, M., Esqueda, A., Bai, F., Steinkellner, H., and Chen, Q. (2016). Plant-produced anti-dengue virus monoclonal antibodies exhibit reduced antibody-dependent enhancement of infection activity. J Gen Virol. 97, 3280-3290.
207. Lai, H., Paul, A. M., Sun, H., He, J., Yang, M., Bai, F., and Chen, Q. (2018). A plant-produced vaccine protects mice against lethal West Nile virus infection without enhancing Zika or dengue virus infectivity. Vaccine 36, 1846-1852.
208. Powell, A. E., Zhang, K., Sanyal, M., Tang, S., Weidenbacher, P. A., Li, S., Pham, T. D., Pak, J. E., Chiu, W., and Kim, P. S. (2020). A single immunization with spike-functionalized ferritin vaccines elicits neutralizing antibody responses against SARS-CoV-2 in mice. bioRxiv.
209. Imai, M., Iwatsuki-Horimoto, K., Hatta, M., Loeber, S., Halfmann, P. J., Nakajima, N., Watanabe, T., Ujie, M., Takahashi, K., Ito, M., et al. (2020). Syrian hamsters as a small animal model for SARS-CoV-2 infection and countermeasure development. Proc Natl Acad Sci USA 117, 16587-16595.
210. Munoz-Fontela, C., Dowling, W. E., Funnell, S. G. P., Gsell, P. S., Riveros-Balta, A. X., Albrecht, R. A., Andersen, H., Baric, R. S., Carroll, M. W., Cavaleri, M., et al. (2020). Animal models for COVID-19. Nature 586, 509-515.
211. Yuan, S., Wang, R., Chan, J. F., Zhang, A. J., Cheng, T., Chik, K. K., Ye, Z. W., Wang, S., Lee, A. C., Jin, L., et al. (2020). Metallodrug ranitidine bismuth citrate suppresses SARS-CoV-2 replication and relieves virus-associated pneumonia in Syrian hamsters. Nat Microbiol 5, 1439-1448.
212. Paul, A. M., Acharya, D., Neupane, B., Thompson, E. A., Gonzalez-Fernandez, G., Copeland, K. M., Garrett, M., Liu, H., Lopez, M. E., de Cruz, M., et al. (2018). Congenital Zika Virus Infection in Immunocompetent Mice Causes Postnatal Growth Impediment and Neurobehavioral Deficits. Front Microbiol 9, 2028.
213. Hurtado, J., Acharya, D., Lai, H., Sun, H., Kallolimath, S., Steinkellner, H., Bai, F., and Chen, Q. (2020). In vitro and in vivo efficacy of anti-chikungunya virus monoclonal antibodies produced in wild-type and glycoengineered Nicotiana benthamiana plants. Plant Biotechnol J 18, 266-273.
214. Paul, A. M., Acharya, D., Duty, L., Thompson, E. A., Le, L., Stokic, D. S., Leis, A. A., and Bai, F. (2017). Osteopontin facilitates West Nile virus neuroinvasion via neutrophil “Trojan horse” transport. Sci Rep 7, 4722.
215. Regoort, M., Reekers, J. A., and Kromhout, J. G. (1989). An unusual cause of an inferior vena cava syndrome. Neth J Surg 41, 92-94.
216. ADDIN EN.REFLIST Huang, F. (2003). Efficient incorporation of CoA, NAD and FAD into RNA by in vitro transcription. Nucleic Acids Res 31, e8.
217. Coleman, T. M., Wang, G., and Huang, F. (2004). Superior 5′ homogeneity of RNA from ATP-initiated transcription under the T7 phi 2.5 promoter. Nucleic Acids Res 32, e14.
218. Silva, L. A., and Dermody, T. S. (2017). Chikungunya virus: epidemiology, replication, disease mechanisms, and prospective intervention strategies. J Clin Invest 127, 737-749.
219. Cherian, N., Bettis, A., Deol, A., Kumar, A., Di Fabio, J. L., Chaudhari, A., Yimer, S., Fahim, R., and Endy, T. (2023). Strategic considerations on developing a CHIKV vaccine and ensuring equitable access for countries in need. NPJ Vaccines 8, 123.
220. de Lima Cavalcanti, T. Y. V., Pereira, M. R., de Paula, S. O., and Franca, R. F. O. (2022). A Review on Chikungunya Virus Epidemiology, Pathogenesis and Current Vaccine Development. Viruses 14.
221. Schott, J. W., Morgan, M., Galla, M., and Schambach, A. (2016). Viral and Synthetic RNA Vector Technologies and Applications. Mol Ther 24, 1513-1527.
222. Pardi, N., Hogan, M. J., Porter, F. W., and Weissman, D. (2018). mRNA vaccines—a new era in vaccinology. Nat Rev Drug Discov 17, 261-279.
223. Jackson, N. A. C., Kester, K. E., Casimiro, D., Gurunathan, S., and DeRosa, F. (2020). The promise of mRNA vaccines: a biotech and industrial perspective. NPJ Vaccines 5, 11.
224. Zhang, C., Maruggi, G., Shan, H., and Li, J. (2019). Advances in mRNA Vaccines for Infectious Diseases. Front Immunol 10, 594.
225. Wang, Z., Yuan, Z., Matsumoto, M., Hengge, U. R., and Chang, Y. F. (2005). Immune responses with DNA vaccines encoded different gene fragments of severe acute respiratory syndrome coronavirus in BALB/c mice. Biochem Biophys Res Commun 327, 130-135.
226. Vogel, A. B., Lambert, L., Kinnear, E., Busse, D., Erbar, S., Reuter, K. C., Wicke, L., Perkovic, M., Beissert, T., Haas, H., et al. (2018). Self-Amplifying RNA Vaccines Give Equivalent Protection against Influenza to mRNA Vaccines but at Much Lower Doses. Mol Ther 26, 446-455.
227. Chen, N., Xia, P., Li, S., Zhang, T., Wang, T. T., and Zhu, J. (2017). RNA sensors of the innate immune system and their detection of pathogens. IUBMB Life 69, 297-304.
228. Berglund, P., Quesada-Rolander, M., Putkonen, P., Biberfeld, G., Thorstensson, R., and Liljestrom, P. (1997). Outcome of immunization of cynomolgus monkeys with recombinant Semliki Forest virus encoding human immunodeficiency virus type 1 envelope protein and challenge with a high dose of SHIV-4 virus. AIDS Res Hum Retroviruses 13, 1487-1495.
229. Pushko, P., Parker, M., Ludwig, G. V., Davis, N. L., Johnston, R. E., and Smith, J. F. (1997). Replicon-helper systems from attenuated Venezuelan equine encephalitis virus: expression of heterologous genes in vitro and immunization against heterologous pathogens in vivo. Virology 239, 389-401.
230. Hariharan, M. J., Driver, D. A., Townsend, K., Brumm, D., Polo, J. M., Belli, B. A., Catton, D. J., Hsu, D., Mittelstaedt, D., McCormack, J. E., et al. (1998). DNA immunization against herpes simplex virus: enhanced efficacy using a Sindbis virus-based vector. J Virol 72, 950-958.
231. Cheng, W. F., Hung, C. H., Chai, C. Y., Hsu, K. F., He, L., Ling, M., and Wu, T. C. (2001). Enhancement of sindbis virus self-replicating RNA vaccine potency by linkage of herpes simplex virus type 1 VP22 protein to antigen. J Virol 75, 2368-2376.
232. Perri, S., Greer, C. E., Thudium, K., Doe, B., Legg, H., Liu, H., Romero, R. E., Tang, Z., Bin, Q., Dubensky, T. W., Jr., et al. (2003). An alphavirus replicon particle chimera derived from venezuelan equine encephalitis and sindbis viruses is a potent gene-based vaccine delivery vector. J Virol 77, 10394-10403.
233. Ljungberg, K., and Liljestrom, P. (2015). Self-replicating alphavirus RNA vaccines. Expert Rev Vaccines 14, 177-194.
234. Pijlman, G. P., Suhrbier, A., and Khromykh, A. A. (2006). Kunjin virus replicons: an RNA-based, non-cytopathic viral vector system for protein production, vaccine and gene therapy applications. Expert Opin Biol Ther 6, 135-145.
235. McCullough, K. C., Bassi, I., Milona, P., Suter, R., Thomann-Harwood, L., Englezou, P., Demoulins, T., and Ruggli, N. (2014). Self-replicating Replicon-RNA Delivery to Dendritic Cells by Chitosan-nanoparticles for Translation In Vitro and In Vivo. Mol Ther Nucleic Acids 3, e173.
236. Englezou, P. C., Sapet, C., Demoulins, T., Milona, P., Ebensen, T., Schulze, K., Guzman, C. A., Poulhes, F., Zelphati, O., Ruggli, N., et al. (2018). Self-Amplifying Replicon RNA Delivery to Dendritic Cells by Cationic Lipids. Mol Ther Nucleic Acids 12, 118-134.
237. Pugachev, K. V., Guirakhoo, F., Ocran, S. W., Mitchell, F., Parsons, M., Penal, C., Girakhoo, S., Pougatcheva, S. O., Arroyo, J., Trent, D. W., et al. (2004). High fidelity of yellow fever virus RNA polymerase. J Virol 78, 1032-1038.
238. King, N. J., and Kesson, A. M. (1988). Interferon-independent increases in class I major histocompatibility complex antigen expression follow flavivirus infection. J Gen Virol 69 (Pt 10), 2535-2543.
239. Mullbacher, A., and Lobigs, M. (1995). Up-regulation of MHC class I by flavivirus-induced peptide translocation into the endoplasmic reticulum. Immunity 3, 207-214.
240. Nazneen, F., Thompson, E. A., Blackwell, C., Bai, J. S., Huang, F., and Bai, F. (2023). An effective live-attenuated Zika vaccine candidate with a modified 5′ untranslated region. NPJ Vaccines 8, 50.
241. Huang, F., Spangler, J. R., and Huang, A. Y. (2017). In vivo cloning of up to 16 kb plasmids in E. coli is as simple as PCR. PLoS One 12, e0183974.
242. Sapkota, K., and Huang, F. (2018). Efficient one-pot enzymatic synthesis of dephospho coenzyme A. Bioorg Chem 76, 23-27.
243. Dhakal, S., Sapkota, K., Huang, F., and Rangachari, V. (2020). Cloning, expression and purification of the low-complexity region of RanBP9 protein. Protein Expr Purif 172, 105630.
244. Suhrbier, A., Jaffar-Bandjee, M. C., and Gasque, P. (2012). Arthritogenic alphaviruses—an overview. Nat Rev Rheumatol 8, 420-429.
245. Staples, J. E., and Fischer, M. (2014). Chikungunya virus in the Americas—what a vectorborne pathogen can do. N Engl J Med 371, 887-889.
246. Tsetsarkin, K. A., Vanlandingham, D. L., McGee, C. E., and Higgs, S. (2007). A single mutation in chikungunya virus affects vector specificity and epidemic potential. PLoS Pathog 3, e201.
247. Gibney, K. B., Fischer, M., Prince, H. E., Kramer, L. D., St George, K., Kosoy, O. L., Laven, J. J., and Staples, J. E. (2011). Chikungunya fever in the United States: a fifteen year review of cases. Clin Infect Dis 52, e121-126.
248. Schwartz, O., and Albert, M. L. (2010). Biology and pathogenesis of chikungunya virus. Nat Rev Microbiol 8, 491-500.
249. Schilte, C., Staikowsky, F., Couderc, T., Madec, Y., Carpentier, F., Kassab, S., Albert, M. L., Lecuit, M., and Michault, A. (2013). Chikungunya virus-associated long-term arthralgia: a 36-month prospective longitudinal study. PLoS Negl Trop Dis 7, e2137.
250. Tandale, B. V., Sathe, P. S., Arankalle, V. A., Wadia, R. S., Kulkarni, R., Shah, S. V., Shah, S. K., Sheth, J. K., Sudeep, A. B., Tripathy, A. S., et al. (2009). Systemic involvements and fatalities during Chikungunya epidemic in India, 2006. J Clin Virol 46, 145-149.
251. Kowalski, P. S., Rudra, A., Miao, L., and Anderson, D. G. (2019). Delivering the Messenger: Advances in Technologies for Therapeutic mRNA Delivery. Mol Ther 27, 710-728.
252. Gomez-Aguado, I., Rodriguez-Castejon, J., Vicente-Pascual, M., Rodriguez-Gascon, A., Solinis, M. A., and Del Pozo-Rodriguez, A. (2020). Nanomedicines to Deliver mRNA: State of the Art and Future Perspectives. Nanomaterials (Basel) 10.
253. Lundstrom, K. (2015). Alphaviruses in gene therapy. Viruses 7, 2321-2333.
254. Berglund, P., Smerdou, C., Fleeton, M. N., Tubulekas, I., and Liljestrom, P. (1998). Enhancing immune responses using suicidal DNA vaccines. Nat Biotechnol 16, 562-565.
255. Geall, A. J., Verma, A., Otten, G. R., Shaw, C. A., Hekele, A., Banerjee, K., Cu, Y., Beard, C. W., Brito, L. A., Krucker, T., et al. (2012). Nonviral delivery of self-amplifying RNA vaccines. Proc Natl Acad Sci USA 109, 14604-14609.
256. Li, Y., Teague, B., Zhang, Y., Su, Z., Porter, E., Dobosh, B., Wagner, T., Irvine, D. J., and Weiss, R. (2019). In vitro evolution of enhanced RNA replicons for immunotherapy. Sci Rep 9, 6932.
257. Hekele, A., Bertholet, S., Archer, J., Gibson, D. G., Palladino, G., Brito, L. A., Otten, G. R., Brazzoli, M., Buccato, S., Bonci, A., et al. (2013). Rapidly produced SAM((R)) vaccine against H7N9 influenza is immunogenic in mice. Emerg Microbes Infect 2, e52.
258. Pang, X., Zhang, M., and Dayton, A. I. (2001). Development of Dengue virus type 2 replicons capable of prolonged expression in host cells. BMC Microbiol 1, 18.
259. Shi, P. Y., Tilgner, M., and Lo, M. K. (2002). Construction and characterization of subgenomic replicons of New York strain of West Nile virus. Virology 296, 219-233.
260. Scholle, F., Girard, Y. A., Zhao, Q., Higgs, S., and Mason, P. W. (2004). trans-Packaged West Nile virus-like particles: infectious properties in vitro and in infected mosquito vectors. J Virol 78, 11605-11614.
261. Jones, C. T., Patkar, C. G., and Kuhn, R. J. (2005). Construction and applications of yellow fever virus replicons. Virology 331, 247-259.
262. Jones, M., Davidson, A., Hibbert, L., Gruenwald, P., Schlaak, J., Ball, S., Foster, G. R., and Jacobs, M. (2005). Dengue virus inhibits alpha interferon signaling by reducing STAT2 expression. J Virol 79, 5414-5420.
263. Xie, X., Zou, J., Shan, C., Yang, Y., Kum, D. B., Dallmeier, K., Neyts, J., and Shi, P. Y. (2016). Zika Virus Replicons for Drug Discovery. EBioMedicine 12, 156-160.
264. Xie, X., Kum, D. B., Xia, H., Luo, H., Shan, C., Zou, J., Muruato, A. E., Medeiros, D. B. A., Nunes, B. T. D., Dallmeier, K., et al. (2018). A Single-Dose Live-Attenuated Zika Virus Vaccine with Controlled Infection Rounds that Protects against Vertical Transmission. Cell Host Microbe 24, 487-499 e485.
265. Ramanathan, M. P., Chambers, J. A., Pankhong, P., Chattergoon, M., Attatippaholkun, W., Dang, K., Shah, N., and Weiner, D. B. (2006). Host cell killing by the West Nile Virus NS2B-NS3 proteolytic complex: NS3 alone is sufficient to recruit caspase-8-based apoptotic pathway. Virology 345, 56-72.
266. Bai, F., and Thompson, E. A. (2021). West Nile Virus (Flaviviridae). In Encyclopedia of Virology (Fourth Edition), D. H. Bamford and M. Zuckerman, eds. (Oxford: Academic Press), pp. 884-890.
267. Xing, H., Xu, S., Jia, F., Yang, Y., Xu, C., Qin, C., and Shi, L. (2020). Zika NS2B is a crucial factor recruiting NS3 to the ER and activating its protease activity. Virus Research 275, 197793.
268. Palacio, N., Dangi, T., Chung, Y. R., Wang, Y., Loredo-Varela, J. L., Zhang, Z., and Penaloza-MacMaster, P. (2020). Early type I IFN blockade improves the efficacy of viral vaccines. J Exp Med 217.
269. Verbeke, R., Hogan, M. J., Lore, K., and Pardi, N. (2022). Innate immune mechanisms of mRNA vaccines. Immunity 55, 1993-2005.
270. Zhong, Z., Portela Catani, J. P., Mc Cafferty, S., Couck, L., Van Den Broeck, W., Gorle, N., Vandenbroucke, R. E., Devriendt, B., Ulbert, S., Cnops, L., et al. (2019). Immunogenicity and Protection Efficacy of a Naked Self-Replicating mRNA-Based Zika Virus Vaccine. Vaccines (Basel) 7.
271. Grant, A., Ponia, S. S., Tripathi, S., Balasubramaniam, V., Miorin, L., Sourisseau, M., Schwarz, M. C., Sanchez-Seco, M. P., Evans, M. J., Best, S. M., et al. (2016). Zika Virus Targets Human STAT2 to Inhibit Type I Interferon Signaling. Cell Host Microbe 19, 882-890.
272. Kumar, A., Hou, S., Airo, A. M., Limonta, D., Mancinelli, V., Branton, W., Power, C., and Hobman, T. C. (2016). Zika virus inhibits type-I interferon production and downstream signaling. EMBO Rep 17, 1766-1775.
273. Lin, S., Yang, S., He, J., Guest, J. D., Ma, Z., Yang, L., Pierce, B. G., Tang, Q., and Zhang, Y. J. (2019). Zika virus NS5 protein antagonizes type I interferon production via blocking TBK1 activation. Virology 527, 180-187.
274. Wang, R., Teng, C., Spangler, J., Wang, J., Huang, F., and Guo, Y. L. (2014). Mouse embryonic stem cells have underdeveloped antiviral mechanisms that can be exploited for the development of mRNA-mediated gene expression strategy. Stem Cells Dev 23, 594-604.
275. Burt, F. J., Chen, W., Miner, J. J., Lenschow, D. J., Merits, A., Schnettler, E., Kohl, A., Rudd, P. A., Taylor, A., Herrero, L. J., et al. (2017). Chikungunya virus: an update on the biology and pathogenesis of this emerging pathogen. Lancet Infect Dis 17, e107-e117.
276. Coirada, F. C., Fernandes, E. R., Mello, L. R., Schuch, V., Soares Campos, G., Braconi, C. T., Boscardin, S. B., and Santoro Rosa, D. (2023). Heterologous DNA Prime-Subunit Protein Boost with Chikungunya Virus E2 Induces Neutralizing Antibodies and Cellular-Mediated Immunity. Int J Mol Sci 24.
277. Schmitz, K. S., Comvalius, A. D., Nieuwkoop, N. J., Geers, D., Weiskopf, D., Ramsauer, K., Sette, A., Tschismarov, R., de Vries, R. D., and de Swart, R. L. (2023). A measles virus-based vaccine induces robust chikungunya virus-specific CD4(+) T-cell responses in a phase II clinical trial. Vaccine 41, 6495-6504.
278. Baker, C., Liu, Y., Zou, J., Muruato, A., Xie, X., and Shi, P. Y. (2020). Identifying optimal capsid duplication length for the stability of reporter flaviviruses. Emerg Microbes Infect 9, 2256-2265.
279. Bai, F., Kong, K. F., Dai, J., Qian, F., Zhang, L., Brown, C. R., Fikrig, E., and Montgomery, R. R. (2010). A paradoxical role for neutrophils in the pathogenesis of West Nile virus. J Infect Dis 202, 1804-1812.
280. D'Angelo, W., Acharya, D., Wang, R., Wang, J., Gurung, C., Chen, B., Bai, F., and Guo, Y. L. (2016). Development of Antiviral Innate Immunity During In Vitro Differentiation of Mouse Embryonic Stem Cells. Stem Cells Dev 25, 648-659.
281. Wang, R., Wang, J., Paul, A. M., Acharya, D., Bai, F., Huang, F., and Guo, Y. L. (2013). Mouse embryonic stem cells are deficient in type I interferon expression in response to viral infections and double-stranded RNA. J Biol Chem 288, 15926-15936.
282. Wang, R., Wang, J., Acharya, D., Paul, A. M., Bai, F., Huang, F., and Guo, Y. L. (2014). Antiviral responses in mouse embryonic stem cells: differential development of cellular mechanisms in type I interferon production and response. J Biol Chem 289, 25186-25198.
283. Angel, M., and Yanik, M. F. (2010). Innate immune suppression enables frequent transfection with RNA encoding reprogramming proteins. PLoS One 5, e11756.
284. Bhalla, N., Sun, C., Metthew Lam, L. K., Gardner, C. L., Ryman, K. D., and Klimstra, W. B. (2016). Host translation shutoff mediated by non-structural protein 2 is a critical factor in the antiviral state resistance of Venezuelan equine encephalitis virus. Virology 496, 147-165.
285. Acharya, D., Paul, A. M., Anderson, J. F., Huang, F., and Bai, F. (2015). Loss of Glycosaminoglycan Receptor Binding after Mosquito Cell Passage Reduces Chikungunya Virus Infectivity. PLoS Negl Trop Dis 9, e0004139.
286. Neupane, B., Acharya, D., Nazneen, F., Gonzalez-Fernandez, G., Flynt, A. S., and Bai, F. (2020). Interleukin-17A Facilitates Chikungunya Virus Infection by Inhibiting IFN-alpha2 Expression. Front Immunol 11, 588382.
287. Haese, N. N., Broeckel, R. M., Hawman, D. W., Heise, M. T., Morrison, T. E., and Streblow, D. N. (2016). Animal Models of Chikungunya Virus Infection and Disease. J Infect Dis 214, S482-S487.
288. Schilte, C., Couderc, T., Chretien, F., Sourisseau, M., Gangneux, N., Guivel-Benhassine, F., Kraxner, A., Tschopp, J., Higgs, S., Michault, A., et al. (2010). Type I IFN controls chikungunya virus via its action on nonhematopoietic cells. J Exp Med 207, 429-442.
289. Berneck, B. S., Rockstroh, A., Fertey, J., Grunwald, T., and Ulbert, S. (2020). A Recombinant Zika Virus Envelope Protein with Mutations in the Conserved Fusion Loop Leads to Reduced Antibody Cross-Reactivity upon Vaccination. Vaccines (Basel) 8.
290. Powers, J. M., Haese, N. N., Denton, M., Ando, T., Kreklywich, C., Bonin, K., Streblow, C. E., Kreklywich, N., Smith, P., Broeckel, R., et al. (2021). Non-replicating adenovirus based Mayaro virus vaccine elicits protective immune responses and cross protects against other alphaviruses. PLoS Negl Trop Dis 15, e0009308.
291. Zang, J., Gu, C., Zhou, B., Zhang, C., Yang, Y., Xu, S., Bai, L., Zhang, R., Deng, Q., Yuan, Z., et al. (2020). Immunization with the receptor-binding domain of SARS-CoV-2 elicits antibodies cross-neutralizing SARS-CoV-2 and SARS-CoV without antibody-dependent enhancement. Cell Discov 6, 61.
292. Liu, X., Drelich, A., Li, W., Chen, C., Sun, Z., Shi, M., Adams, C., Mellors, J. W., Tseng, C. T., and Dimitrov, D. S. (2020). Enhanced elicitation of potent neutralizing antibodies by the SARS-CoV-2 spike receptor binding domain Fc fusion protein in mice. Vaccine 38, 7205-7212.
293. Dent, M., Hurtado, J., Paul, A. M., Sun, H., Lai, H., Yang, M., Esqueda, A., Bai, F., Steinkellner, H., and Chen, Q. (2016). Plant-produced anti-dengue virus monoclonal antibodies exhibit reduced antibody-dependent enhancement of infection activity. J Gen Virol 97, 3280-3290.
294. Lai, H., Paul, A. M., Sun, H., He, J., Yang, M., Bai, F., and Chen, Q. (2018). A plant-produced vaccine protects mice against lethal West Nile virus infection without enhancing Zika or dengue virus infectivity. Vaccine 36, 1846-1852.
295. Acharya, D., Wang, P., Paul, A. M., Dai, J., Gate, D., Lowery, J. E., Stokic, D. S., Leis, A. A., Flavell, R. A., Town, T., et al. (2017). Interleukin-17A Promotes CD8+ T Cell Cytotoxicity To Facilitate West Nile Virus Clearance. J Virol 91.
296. Paul, A. M., Acharya, D., Le, L., Wang, P., Stokic, D. S., Leis, A. A., Alexopoulou, L., Town, T., Flavell, R. A., Fikrig, E., et al. (2016). TLR8 Couples SOCS-1 and Restrains TLR7-Mediated Antiviral Immunity, Exacerbating West Nile Virus Infection in Mice. J Immunol 197, 4425-4435.
297. Bai, F., Town, T., Qian, F., Wang, P., Kamanaka, M., Connolly, T. M., Gate, D., Montgomery, R. R., Flavell, R. A., and Fikrig, E. (2009). IL-10 signaling blockade controls murine West Nile virus infection. PLoS Pathog 5, e1000610.
298. Town, T., Bai, F., Wang, T., Kaplan, A. T., Qian, F., Montgomery, R. R., Anderson, J. F., Flavell, R. A., and Fikrig, E. (2009). Toll-like receptor 7 mitigates lethal West Nile encephalitis via interleukin 23-dependent immune cell infiltration and homing. Immunity 30, 242-253.
299. Paul, A. M., Acharya, D., Neupane, B., Thompson, E. A., Gonzalez-Fernandez, G., Copeland, K. M., Garrett, M., Liu, H., Lopez, M. E., de Cruz, M., et al. (2018). Congenital Zika Virus Infection in Immunocompetent Mice Causes Postnatal Growth Impediment and Neurobehavioral Deficits. Front Microbiol 9, 2028.
300. Hurtado, J., Acharya, D., Lai, H., Sun, H., Kallolimath, S., Steinkellner, H., Bai, F., and Chen, Q. (2020). In vitro and in vivo efficacy of anti-chikungunya virus monoclonal antibodies produced in wild-type and glycoengineered Nicotiana benthamiana plants. Plant Biotechnol J 18, 266-273.
301. Paul, A. M., Acharya, D., Duty, L., Thompson, E. A., Le, L., Stokic, D. S., Leis, A. A., and Bai, F. (2017). Osteopontin facilitates West Nile virus neuroinvasion via neutrophil “Trojan horse” transport. Sci Rep 7, 4722.
302. Regoort, M., Reekers, J. A., and Kromhout, J. G. (1989). An unusual cause of an inferior vena cava syndrome. Neth J Surg 41, 92-94.

1. A self-amplifying mRNA vaccine, comprising:
a self-amplifying backbone of a live attenuated virus, Z7, having a hairpin loop insert, and wherein at least one or more structural genes of the live attenuated virus have been replaced with at least one target antigen-encoding nucleic acid sequence.

2. The vaccine of claim 1, wherein the live attenuated virus comprises the first 50 amino acids of a capsid (C) protein and the last 30 amino acids of an envelope glycoprotein (E).

3. The vaccine of claim 1, wherein the hairpin loop insert comprises a nucleic acid sequence comprising 50 nucleotides.

4. The vaccine of claim 1, wherein the hairpin loop insert is an RNA sequence CGUUCCAACCACUGACUCGAAAGAGUCAGUGGUUGGAACGCGCAGGUGCC (SEQ ID NO: 10), in the 5′ untranslated region of the virus.

5. The vaccine of claim 1, wherein the hairpin loop insert comprises a paired stem region and an unpaired middle loop, wherein the stem region comprises greater than 50% cytosine-guanine nucleotide pairs, based on the total combined number of adenine-uracil nucleotide pairs and cytosine-guanine nucleotide pairs.

6. The vaccine of claim 1, further comprising an encapsulating component for delivery, selected from the group consisting of a lipid-nanoparticle and a polymer.

7. The vaccine of claim 1, wherein the target antigen is derived from a viral pathogen or a cancer specific antigen.

8. The vaccine of claim 1, wherein the target antigen is selected from antigens of viral pathogens of the families of Togaviridae, Coronaviridae, Flaviviridae, Piconaviridae, Paramyxoviridae, Rhabdoviridae, Filoviridae, Bunyaviridae, Orthomyxoviridae, Papillomaviridae, Herpesviridae, Poxviridae, and Retroviridae.

9. The vaccine of claim 1, wherein target antigen is derived from an RNA virus selected from the group consisting of an alphavirus, a flavivirus, a picornavirus, a coronavirus, a retrovirus, a paramyxovirus, a rhabdovirus, an orthomyxovirus, a filovirus, a rotavirus, an orthopneumovirus, a togavirus, and an arterivirus.

10. The vaccine of claim 9, wherein the RNA virus is an alphavirus selected from the group consisting of Chikungunya virus, Ross River Virus, Sindbis virus, Mayaro virus, Semliki Forest virus, Eastern equine encephalitis virus, Western equine encephalitis virus, Venezuelan equine encephalitis virus, and Aura virus.

11. The vaccine of claim 9, wherein the RNA virus is a flavivirus is selected from the group consisting of West Nile Virus, Dengue Virus, Tick-borne encephalitis virus, Yellow Fever Virus, and Zika Virus.

12. The vaccine of claim 1, wherein the target antigen is derived from a Chikungunya virus.

13. The vaccine of claim 1, wherein the live attenuated virus is Zika virus.

14. The vaccine of claim 13, wherein the Zika virus is the Cambodian strain FSS13025.

15. The vaccine of claim 1, wherein the target antigen is a viral structural protein on an outer surface of a viral particle.

16. The vaccine of claim 15, wherein the viral structural protein is selected from the group consisting of spike proteins and envelope proteins.

17. The vaccine of claim 16, wherein the viral structural protein is a protein that interacts with host cell receptors to facilitate the viral particle gaining entry into the host cell.

18. The vaccine of claim 1, wherein the target antigen is a cancer specific antigen or a tumor specific antigen.

19. The vaccine of claim 18, wherein the cancer or tumor specific antigen is located on an outer surface of a cancer or tumor cell.

20. The vaccine of claim 18, wherein the tumor specific antigen is selected from the group consisting of CA-125, CA15-3, CA19-9, hCG, beta-hCG, PSA, Calcitonin, SCC, URLC10AFP, CD19, TRAC, TCRB, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, epithelial tumor antigen, mesothelin, IL-11Ra, PSCA, PRSS21, VEGFR1, VEGFR2, LewisY, CD24, PDGFR-beta, SSEA-4, CD20, folate receptor alpha, ERBB2 (Her2/neu), MUC-1, MUC-2, MUC16, EGFR, NCAM, prostase, PAP, ELF2M, Ephrin B2, IGF-I receptor, CAIX, LMP2, gp 100, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, WT1HPV16 E7, HPV E6, E7, MAGE A1, ETV6-AML, sperm protein 17, XAGE1, Tie 2, MAD-CT-1, MAD-CT-2, Fos-related antigen 1, p53, p53 mutant, protein, surviving, telomerase, PCTA-1/Galectin 8, MelanA/MART1, Ras mutant, hTERT, sarcoma translocation breakpoints, gp100, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, MART-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3c, CD4, CD5, and CD7.
